(19) United States
(12) Patent Application Publication
(10) Pub. No.: US 2023/0029042 A1
(43) Pub. Date: Jan. 26, 2023

(54) WEARABLE SYSTEM FOR THE EAR

(71) Applicant: Earlens Corporation, Menlo Park, CA (US)

(72) Inventors: William FACTEAU, Atherton, CA (US); Lawrence W. ARNE, Palo Alto, CA (US); Suzanne LEVY, Belmont, CA (US); Drew DUNDAS, San Anselmo, CA (US); Greg KOSKOWICH, Pleasanton, CA (US); Pragati Rao MANDIKAL VASUKI, Menlo Park, CA (US); Srinivasamurthy Ravi PRAKASH, Saratoga, CA (US); Rodney C. PERKINS, Woodside, CA (US); Stuart W. WENZEL, San Carlos, CA (US)

(21) Appl. No.: 17/894,889

(22) Filed: Aug. 24, 2022

Related U.S. Application Data
(63) Continuation of application No. PCT/US21/19176, filed on Feb. 23, 2021.
(60) Provisional application No. 62/982,996, filed on Feb. 28, 2020.
(60) Provisional application No. 62/986,412, filed on Mar. 6, 2020.

(51) Int. Cl. A61B 5/00; H04R 1/10; A61B 5/11; A61B 5/318; A61B 5/1455; A61B 5/0205

(52) U.S. Cl. CPC A61B 5/6817; H04R 1/10; A61B 5/1114; A61B 5/1102; A61B 5/318; A61B 5/14551; A61B 5/02055; A61B 5/0002; A61B 2562/0271; A61B 2562/0204; A61B 5/02416

(57) ABSTRACT

Methods of measuring biometric characteristics using a sensor positioned in an ear canal of a user are provided. The sensor is positioned on or connected to an ear tip, a contact hearing device, or one or more components thereof. One or more biometric signals may be sensed from the sensor. The biometric characteristic of the user is measured or derived from these sensed signals, and include but are not limited to the temperature of the user, acoustic signal(s) from the user, movement(s) of the user, a ballistocardiogram, an electrocardiogram, oxygen saturation, and blood pressure.

### CROSS-REFERENCE

This application is a continuation of PCT Application No. PCT/US21/19176, filed Feb. 23, 2021; which claims priority to U.S. Provisional Application Nos. 62/982,996, filed Feb. 28, 2020, and 62/986,412, filed Mar. 6, 2020; all of which are incorporated herein by reference in their entirety.

### BACKGROUND

The present disclosure relates to medical systems, devices, and methods, particularly for measuring biometric and other data from the ear canal of a subject and similar anatomical features.

### SUMMARY

In an aspect of the present disclosure, methods of measuring a biometric characteristic of a user are provided. A sensor may be positioned in the ear canal of a user, wherein the sensor is positioned on or connected to an ear tip, contact hearing device or one or more components thereof. One or more biometric signals may be sensed from the sensor. The biometric characteristic of the user may be measured or derived from the one or more biometric signals.

In another aspect of the present disclosure, method of measuring the temperature of a user are provided. A sensor may be positioned in the ear canal of a user, wherein the sensor is positioned on or connected to an ear tip, contact hearing device or one or more components thereof. One or more biometric signals may be sensed from the sensor. The temperature of the user may be measured or derived from the one or more biometric signals.

In some embodiments, the sensor is a temperature sensor.

In another aspect of the present disclosure, methods of measuring sounds conducted by the bones of a user are provided. A sensor may be positioned in the ear canal of a user, wherein the sensor is positioned on or connected to an ear tip, contact hearing device or one or more components thereof. One or more acoustic signals may be sensed from said sensor.

In some embodiments, the sensor is an acoustic sensor.

In some embodiments, the sensor is a microphone.

In another aspect of the present disclosure, methods of measuring movements of a user are provided. A sensor may be positioned in the ear canal of a user, wherein the sensor is positioned on or connected to an ear tip, contact hearing device or one or more components thereof. One or more movement related signals from the sensor may be sensed. The sensed signal may be used to measure or derive the movement of the user or a portion of the user's body.

In some embodiments, the sensor is an accelerometer.

In another aspect of the present disclosure, methods of generating a ballistocardiogram for a user may be provided. A sensor may be positioned in the ear canal of a user, wherein the sensor is positioned on or connected to an ear tip, contact hearing device or one or more components thereof. One or more biometric characteristics of the user may be sensed from the sensor. The sensed biometric characteristics may be used to generate a ballistocardiogram for the user.

In some embodiments, the sensor is an accelerometer.

In another aspect of the present disclosure, methods of generating an electrocardiogram for a user are provided. A sensor may be positioned in the ear canal of a user, wherein the sensor is positioned on or connected to an ear tip, contact hearing device or one or more components thereof. One or more biometric characteristics of the user may be sensed from the sensor. The sensed biometric characteristics may be used to generate an electrocardiogram for the user.

In some embodiments, the sensor is an accelerometer.

In another aspect of the present disclosure, method of measuring the oxygen saturation level of a user are provided. A sensor may be positioned in the ear canal of a user, wherein the sensor is positioned on or connected to an ear tip, contact hearing device or one or more components thereof. One or more biometric characteristics of the user may be sensed from the sensor. The sensed biometric characteristics may be used to determine the oxygen saturation level of a user.

In some embodiments, the sensor is a pulse oximetry sensor.

In another aspect of the present disclosure, methods of measuring the blood pressure of a user are provided. A sensor may be positioned in the ear canal of a user, wherein the sensor is positioned on or connected to an ear tip, contact hearing device or one or more components thereof. One or more biometric characteristics of the user may be sensed from the sensor. The sensed biometric characteristics may be used to determine the blood pressure of a user.

In some embodiments, wherein the sensor is a photoplethysmography sensor.

### BRIEF DESCRIPTION OF THE DRAWINGS

The foregoing and other objects, features, and advantages of embodiments of the present inventive concepts will be apparent from the more particular description of preferred embodiments, as illustrated in the accompanying drawings in which like reference characters refer to the same or like elements. The drawings are not necessarily to scale, emphasis instead being placed upon illustrating the principles of the preferred embodiments.

FIG. 1 is a cutaway view of an ear canal showing a contact hearing system according to the present invention wherein at least a portion of the contact hearing system is positioned in the ear canal.

FIG. 2 is a block diagram of a contact hearing system according to the present invention.

FIG. 3 is a top view of a contact hearing device according to the present invention.

FIG. 4 is a bottom view of a contact hearing device according to the present invention.

FIG. 5 is a side view of a portion of a contact hearing device, including a drive post and umbo lens, according to the present invention.

FIG. 6 is a cutaway view of an ear canal illustrating the positioning of a contact hearing device according to the present invention.

FIG. 7 illustrates a processor and ear tip according to the present invention.

FIG. 8 is a side perspective view of a transmit coil for use in an ear tip according to the present invention.

FIG. 9 is an end view of an ear tip according to the present invention.

FIG. 10 is a cut away side view of an ear tip according to the present invention.

FIG. 10A is a cut away side view of an ear tip according to the present invention.

FIG. 11 is an end view of an ear tip according to the present invention.

FIG. 12 is a cut away side view of an ear tip according to the present invention.

FIG. 12A is a cut away side view of an ear tip according to the present invention.

FIG. 13A is a top perspective view of a charging station for use in charging processors.

FIG. 13B is a back perspective view of a charging station for use in charging processors.

FIG. 14 is a block diagram of an inductively coupled contact hearing system, including a contact hearing device, according to the present invention.

FIG. 14A is a block diagram of an inductively coupled contact hearing system according to the present invention.

FIG. 15 is a block diagram of a contact hearing system, including an ear tip and contact hearing device according to the present invention.

FIG. 16 is a block diagram of a contact hearing system which is adapted for communication with external devices according to the present invention.

FIG. 17 is a block diagram of a contact hearing device according to the present invention.

FIG. 18 is a diagram of a rectifier circuit for use in a contact hearing system according to the present invention.

FIG. 18A is a diagram of a rectifier circuit for use in a contact hearing system according to the present invention.

FIG. 19 is a diagram of a rectifier and converter circuit for use in a contact hearing system according to the present invention.

FIG. 20 is a diagram of a rectifier and converter circuit for use in a contact hearing system according to the present invention.

FIG. 21 is a diagram of a portion of a contact hearing device according to the present invention.

FIG. 21A is a diagram of a portion of a contact hearing device according to the present invention.

FIG. 22 is a circuit diagram of transmitter and receiver components of a contact hearing system according to embodiments of the present invention.

FIG. 22A is a circuit diagram of transmitter and receiver components of a contact hearing system according to embodiments of the present invention.

FIG. 23 is a circuit diagram of components of a receiver for use in a contact hearing system according to the present invention.

FIG. 24 is a circuit diagram of components of a receiver for use in a contact hearing system according to the present invention.

FIG. 25 is a circuit diagram of components of a transmitter for use in a contact hearing system according to the present invention.

FIG. 26 is a circuit diagram of components of a transmitter for use in a contact hearing system according to the present invention.

FIG. 27 is a circuit diagram of components of a transmitter for use in a contact hearing system according to the present invention.

FIG. 28 is an illustration of a circuit wherein the DSM input is the delta-Sigma modulator output signal used to modulate a carrier signal.

FIG. 29 is a system model of a system according to the present invention, including transmission and receive tank circuits and a detector circuit.

FIG. 30 illustrates the time domain waveform when the added carrier clock is the same size as the delta sigma signal mixed with the carrier clock.

FIG. 31 illustrates the resulting waveform with a 95% delta sigma with the added clock.

FIG. 32 is a graph of output in dB SPL as a function of transmit coil to receive coil distance.

FIGS. 33-35 illustrate various transmit coil vs receive coil alignments according to the present invention.

FIG. 36 illustrates a receiver according to the present invention wherein a Villard circuit is used to demodulate the received signal.

FIG. 37 illustrates a receiver according to the present invention wherein a Greinacher circuit is used to demodulate the received signal.

FIG. 38 is a side view of a transmit coil for use in an ear tip according to the present invention.

FIG. 39 is a top view of a transmit coil for use in an ear tip according to the present invention.

FIG. 40 is a side perspective view of a transmit coil for use in an ear tip according to the present invention.

FIG. 41 is an end view of an ear tip according to the present invention.

FIG. 42 is an end view of an ear tip according to the present invention.

FIG. 43 is a side view of an ear tip assembly according to the present invention.

FIG. 44 is a top and side view of a receive coil according to the present invention.

FIG. 45 is a perspective view of a receive coil according to the present invention.

FIG. 46A is a perspective view of a receive circuit assembly according to the present invention.

FIG. 46B is a perspective exploded view of a receive coil according to the present invention.

FIG. 47 illustrates a receiver according to the present invention wherein a Greinacher circuit is used to demodulate the received signal.

FIG. 48 illustrates a receiver according to the present invention wherein a Greinacher circuit is used to demodulate the received signal.

FIG. 49 is an illustration of a circuit wherein the DSM input is the delta-Sigma modulator output signal used to modulate a carrier signal.

FIG. 50 is an illustration of a circuit wherein the DSM input is the delta-Sigma modulator output signal used to modulate a carrier signal.

FIG. 51 is a system model of a system according to the present invention, including transmission and receive tank circuits and a detector circuit.

FIG. 52 is a graph showing passband tuning according to the present invention for a transmit circuit and a receive circuit according to the present invention.

FIG. 53 shows a hearing system conFIG.d in accordance with embodiments of the present invention.

FIG. 54 shows an isometric view of the medial ear canal assembly of the hearing system of FIG. 53 in accordance with embodiments of the present invention.

FIG. 55 shows a top view of the medial ear canal assembly of the hearing system of FIG. 53 in accordance with embodiments of the present invention.

FIG. 56 shows an exploded view of a medial ear canal assembly and its method of assembly, in accordance with embodiments of the present invention.

FIG. 57 is an isometric Top view of a medial ear canal assembly in accordance with embodiments of the present invention.

FIG. 58 is an isometric bottom view of a medial ear canal assembly in accordance with embodiments of the present invention.

FIG. 59 shows a medial ear canal assembly in accordance with embodiments of the present invention.

FIG. 60 shows an isometric view of a medial ear canal assembly including a drug delivery reservoir in accordance with embodiments of the present invention.

FIG. 61 shows an isometric view of a lateral ear canal assembly in accordance with embodiments of the present invention.

FIG. 62 is an isometric top view of a medial ear canal assembly in accordance with embodiments of the present invention.

FIG. 63 is an isometric bottom view of a medial ear canal assembly in accordance with embodiments of the present invention.

### DETAILED DESCRIPTION OF THE INVENTION

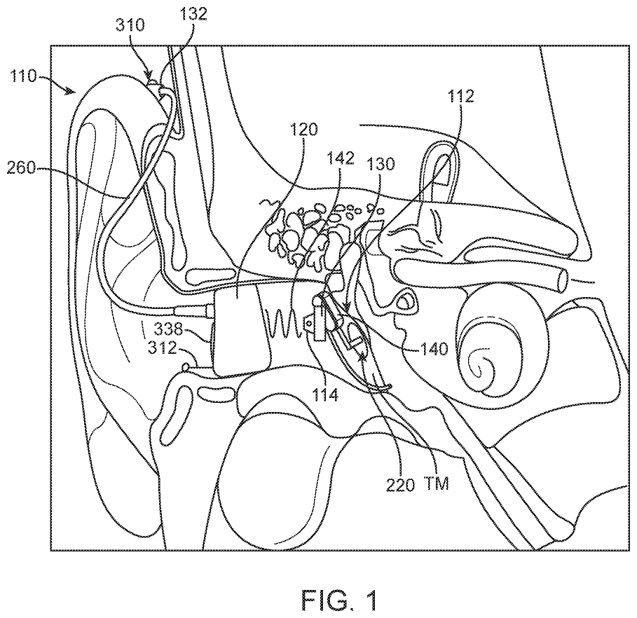

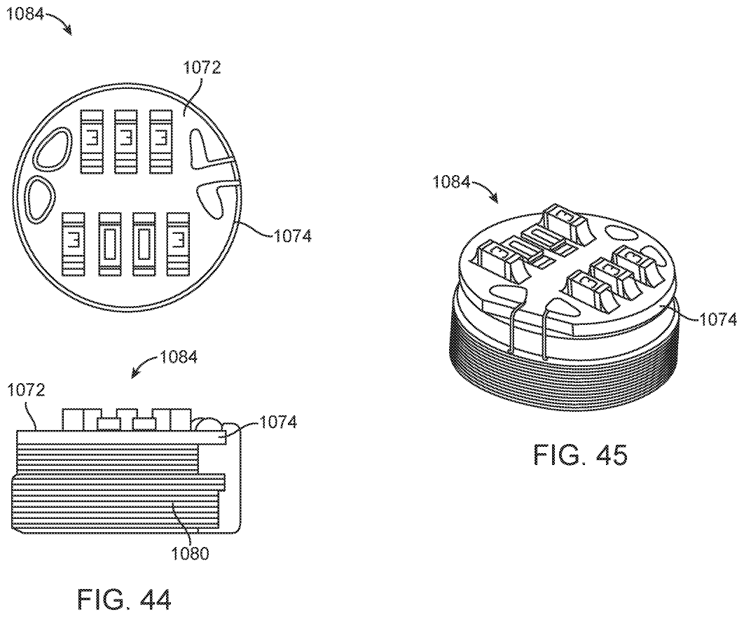

FIG. 1 is a cutaway view of an ear canal showing a contact hearing system 110 for use in systems and methods according to the present invention, wherein at least a portion of the contact hearing system 110 is positioned in the ear canal. In embodiments of the invention, contact hearing system 110 may be referred to as a smartlens system or smartlens. In embodiments of the invention, contact hearing system 110 may comprise a contact hearing system using electromagnetic waves to transmit information and/or power from ear tip 120 to the contact hearing device 112. In embodiments of the invention, contact hearing system 110 may comprise a contact hearing system using inductive coupling to transmit information and/or power from ear tip 120 to contact hearing device 112. In FIG. 1, contact hearing system 110 includes Audio processor 132, which audio processor may include at least one external microphone 310. Audio processor 132 may be connected to an ear tip 120 by cable 260, which is adapted to transmit signals from audio processor 132 to ear tip 120. Ear tip 120 may further include canal microphone 312 and at least one acoustic vent 338. Ear tip 120 may be an ear tip which radiates electromagnetic waves 142 in response to signals from audio processor 132. Electromagnetic signals radiated by ear tip 120 may be received by contact hearing device 112, which may comprise receive coil 130, microactuator 140, and umbo platform 220. As used herein, receive coil 130 may comprise receive circuit assembly 1084 as illustrated in FIGS. 44-46.

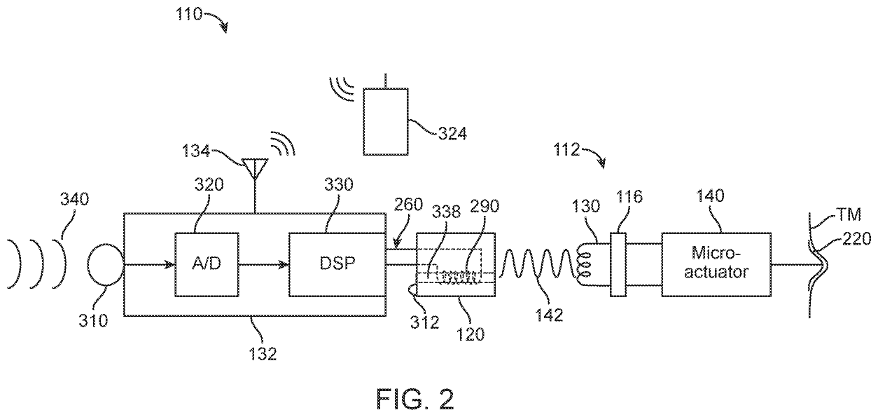

FIG. 2 is a block diagram of a contact hearing system 110 for use in methods and apparatus according to the present invention. In embodiments of the invention, at least a portion of contact hearing system 110 is positioned in the ear canal of a user. In FIG. 2, ambient sound 340 may be received by external microphone 310 of audio processor 132, which then processes the received sound by passing it through processing circuitry, which may include analog to digital converter 320 and digital signal processor 330. The output of audio processor 132 may be transmitted to an ear tip 120 by cable 260. Signals transmitted to ear tip 120 may then be transmitted to contact hearing device 112 by, for example, causing transmit coil 290 to radiate electromagnetic waves 142. In embodiments of the invention, contact hearing device 112 may include receive coil 130, microactuator 140, and umbo lens 220. Information contained in electromagnetic waves 142 received by receive coil 130 may be transmitted through demodulator 116 to microactuator 140, moving umbo lens 220. In embodiments of the invention, the signal transmitted to ear tip 120 may be a signal representative of the received audio signal which may then be transmitted to contact hearing device 112. In embodiments of the invention, transmit coil 290 may be wound around an acoustic vent 338 in ear tip 120. In embodiments of the invention, acoustic vent 338 may be formed as a passage through a ferrite material or a ferromagnetic material. As used herein ferrite material may be any ferromagnetic material. In embodiments of the invention, transmit coil 290 may be wound around ferrite material positioned in ear tip 120. In embodiments of the invention, contact hearing system 110 may include one or more external communication and control devices 324, such as, for example, a cell phone. In embodiments of the invention, audio processor 132 may communicate with external communication and control devices 324 by, for example, using audio processor antenna 134.

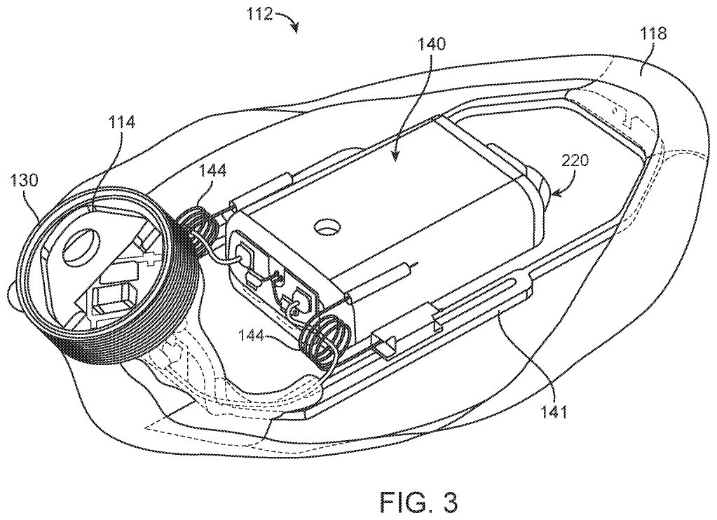

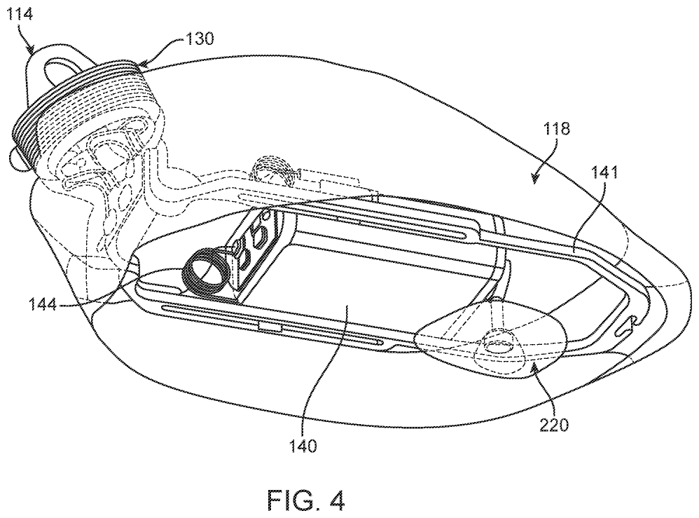

FIG. 3 is atop view of a contact hearing device 112 according to the present invention. FIG. 4 is a bottom view of a contact hearing device 112 according to the present invention. The contact hearing device 112 illustrated in FIGS. 3 and 4 includes a receive coil 130, a microactuator 140, an umbo lens 220, a support structure 141, and springs 144. In the embodiment illustrated in FIGS. 3 and 4, microactuator 140 is connected to support structure 141 by springs 144. In embodiments of the invention, contact hearing device 112 may further include a sulcus platform 118, which may also be referred to as a mounting platform, connected to support structure 141 and adapted to assist in positioning contact hearing device 112 in the ear canal of a user. In embodiments of the invention, contact hearing device 112 may further include grasping tab 114.

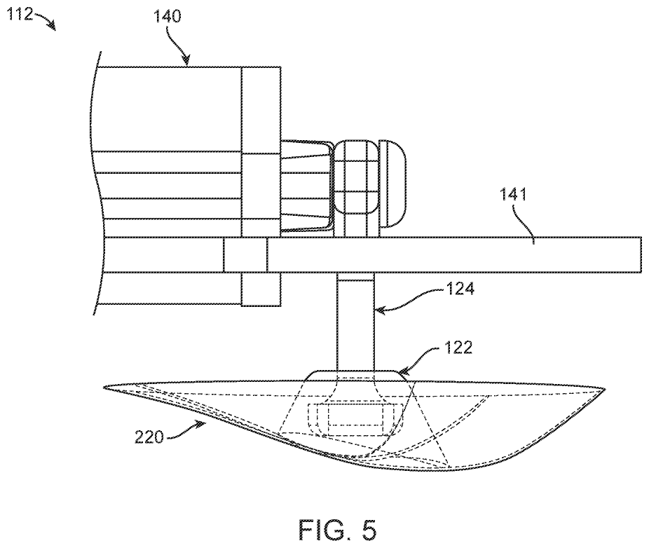

FIG. 5 is a side view of a portion of a contact hearing device 112 according to the present invention, including a drive post 124 and umbo lens 220. In FIG. 5, contact hearing device 112, including a drive post 124 and umbo lens 220. In FIG. 5, drive post 124 may be attached to umbo lens 220 by adhesive 122. Drive post 124 may be attached to the output of microactuator 140, which is supported on contact hearing device 112 by support structure 141.

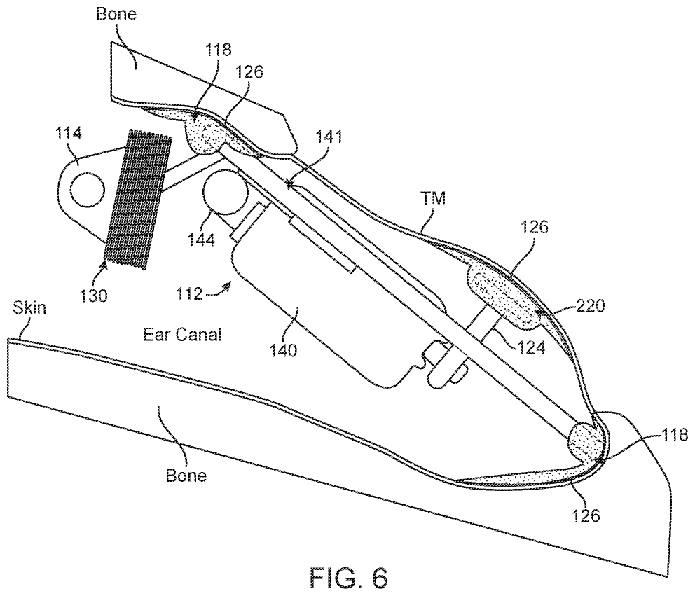

FIG. 6 is a cutaway view of an ear canal illustrating the positioning of a contact hearing device 112 according to the present invention. In the embodiment of FIG. 6, contact hearing device 112 is positioned at a medial end of the ear canal, proximate the tympanic membrane of the user. Contact hearing device 112 includes a receive coil 130 positioned at a medial end thereof. In embodiments of the invention, receive coil 130 may be positioned to receive signals from an ear tip (not shown) positioned in the ear canal lateral to the position of contact hearing device 112. In embodiments of the invention, signals received by receive coil 130 may be transmitted to microactuator 140 to move drive post 124 which is connected to the user's tympanic membrane through umbo lens 220. Umbo lens 220 may be in direct physical contact with the tympanic membrane or a thin layer of oil 126 may be used between umbo lens 220 and the user's tympanic membrane. Sulcus platform 118 may be used to properly position contact hearing device 112 in the user's ear canal through contact with a skin layer which lines the ear canal. Sulcus platform 118 may be in direct contact with the skin of the ear canal or a thin layer of oil 126 may be used between sulcus platform 118 and the skin of the ear canal. In embodiments of the invention contact hearing device 112 may further include support structure 141, grasping tab 114, and springs 144.

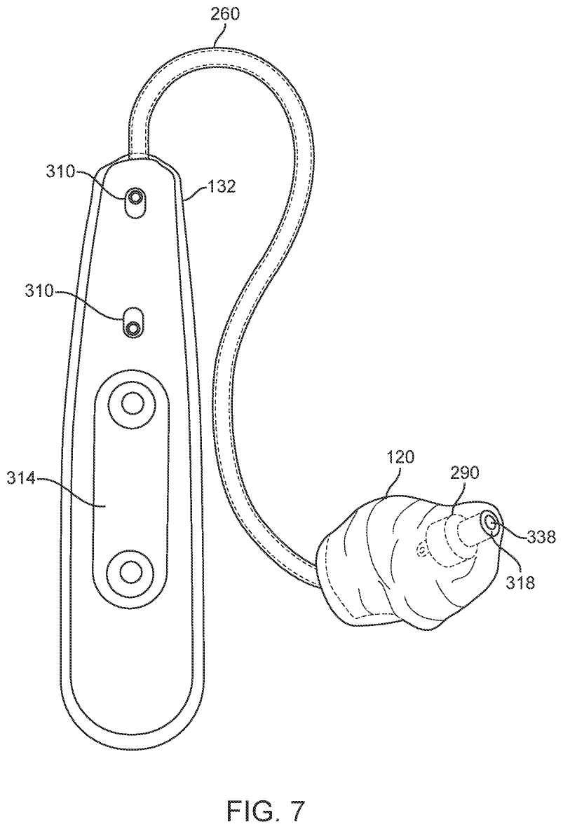

FIG. 7 illustrates an audio processor 132 and ear tip 120 according to the present invention. Ear tip 120 may, in some embodiments of the invention, be referred to as a mag tip or magnetic tip. In the embodiment of FIG. 7, audio processor 132 may include external microphones 310 and volume/control switch 314. In embodiments of the invention, ear tip 120 may include a transmit coil 290 which may include ferrite core 318. In embodiments of the invention, ear tip 120 may include an acoustic vent which may pass through transmit coil 290 and/or through ferrite core 318

FIG. 8 is a side perspective view of a transmit coil 290 for use in an ear tip 120 according to the present invention. In the embodiment of FIG. 8, transmit coil 290 includes coil winding 316 which is wound around ferrite core 318. In embodiments of the invention, transmit coil 290 may further include acoustic vent 338. In embodiments of the invention, transmit coil 290 may further include transmit electronics 342. In embodiments of the invention, transmit coil 290 may be connected to audio processor 132 by cable 260.

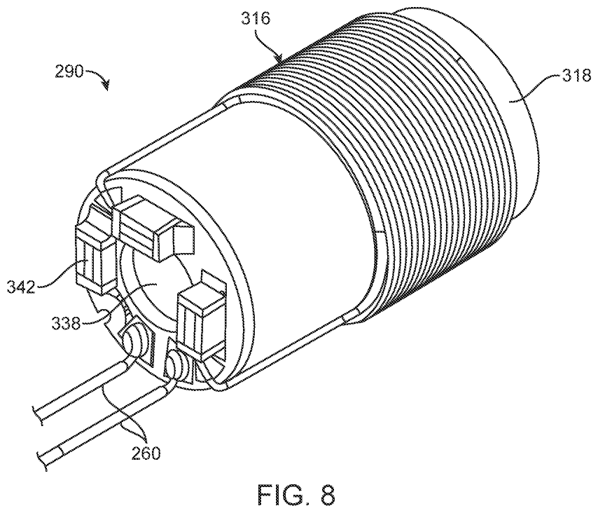

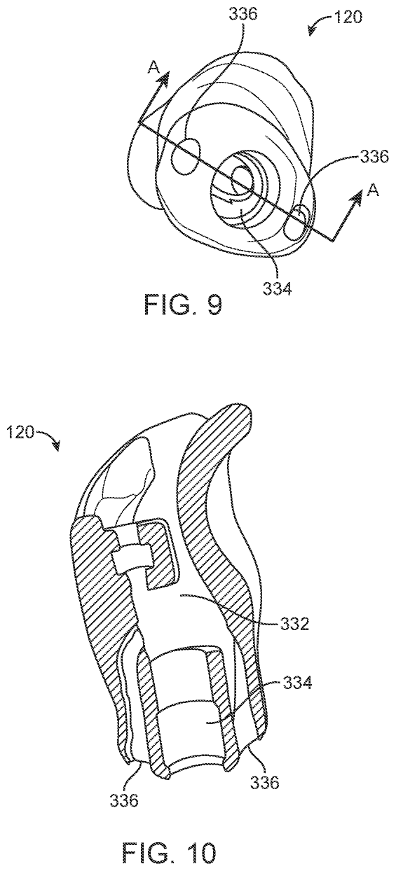

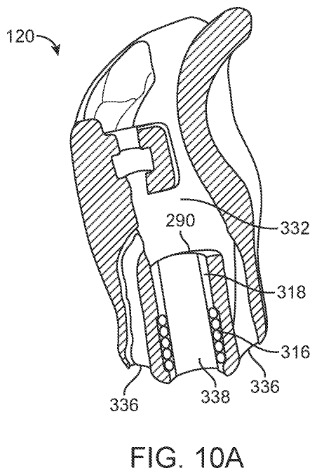

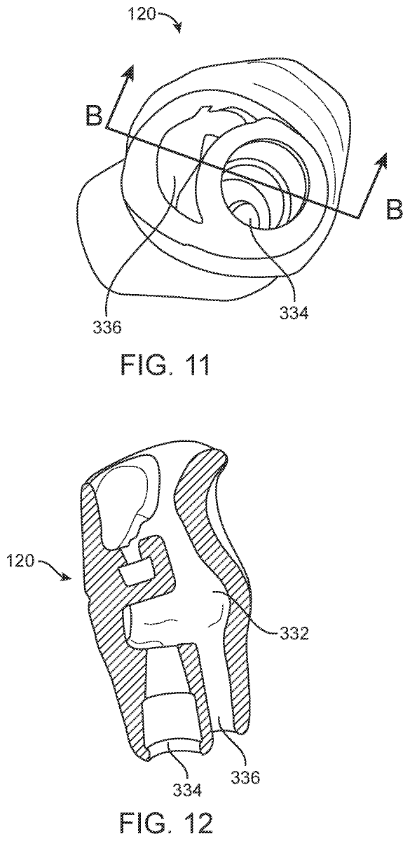

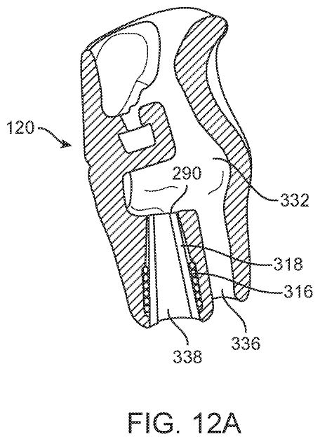

FIG. 9 is an end view of an ear tip 120 according to the present invention. FIGS. 10 and 10A are cut away side views of an ear tip according to the present invention. FIG. 11 is an end view of an ear tip 120 according to the present invention. FIGS. 12 and 12A are cut away side views of an ear tip 120 according to the present invention. In the embodiments of FIGS. 9, 10, 10A, 11, 12 and 12A ear tip 120 includes mounting recess 334, which is adapted to receive transmit coil 290 (shown in FIGS. 10A and 12A). In the embodiments of FIGS. 9-12, ear tip 120 further includes at least one secondary acoustic vent 336. In embodiments of the invention, secondary acoustic vents are adapted to work in conjunction with acoustic vent 338 in transmit coil 290 to reduce the overall acoustic mass of the ear tip. In embodiments of the invention, secondary acoustic vents 336 combine at central chamber 332 which has a larger cross section than the combined cross section of secondary acoustic vents 336. In embodiments of the invention, secondary acoustic vents 336 and acoustic vent 338 combine at central chamber 332 which has a larger cross section than the combined cross section of secondary acoustic vents 336 and acoustic vent 338.

In embodiments of the invention, the total combined acoustic mass (including the acoustic mass of acoustic vent 338 through ferrite core 318 of transmit coil 290, the acoustic mass of any secondary acoustic vents 336 and the acoustic mass of central chamber 332) will not exceed 2000 Kg/m⁴. In embodiments of the invention, the acoustic mass may be defined as the impeding effect of inertia upon the transmission of sound in a conduit, equal in a tubular conduit (as an organ pipe) to the mass of the vibrating medium divided by the square of the cross section. It may also be the acoustic analogue of alternating-current-circuit inductance (called also inertance). In an ear tip which incorporates one or more acoustic vents, the acoustic mass may be representative of the resistance to the flow of air through the ear tip. The acoustic impedance (Z) is frequency specific and relates to the acoustic mass (or inertance, L) as a function of frequency Z=jwL. Acoustic mass may be a function of the cross section of any acoustic vents in an ear tip. Acoustic mass may be a function of the effective length of the acoustic vents in an ear tip. A higher acoustic mass may be perceived by the hearing aid user in a fashion similar to what would be perceived when talking with one's fingers in the ear canals. Thus, a higher acoustic mass effect may be perceived to result in altering the hearing aid user's voice in ways which the hearing aid user finds to be bothersome or unacceptable.

For an even straight tube, the acoustic mass is given by the simple equation:

$$\frac{ρ 1}{A}$$

Where ρ is the density of air (in kg/m3), 1 is the length of the tube, and A is the cross sectional area along the open bore.

For complex openings, the acoustic mass can be described as the integral of the density of air (ρ) divided by the open cross sectional area along the length of the light tip:

$${{\int }_{x = 0}}^{tip length} \frac{ρ}{{Area}_{x}} dx$$

Which can be estimated by dividing the tip along its length into n cross sections and summing each open area as follows:

$${\sum }_{i = 0}^{n} \frac{ρ}{A r e {a}_{i}} x Δ 1$$

Where:

$$Δ 1 = \frac{tip length}{n}$$

In one embodiment the present invention is directed to an ear tip having a proximal end and a distal end, the ear tip including: a transmit coil, the transmit coil including a core of a ferromagnetic material, the ferromagnetic core having a central channel there through, a distal end of the ferromagnetic core positioned at a first opening in a distal end of the ear tip; a passage extending from an opening at a proximal end of the ear tip to the distal end of the ear tip, the passage ending at a second opening in the distal end of the ear tip, wherein a proximal end of the central channel is connected to the passage. In embodiments of the present invention, the combination of the central channel and the passage act as an acoustic vent, allowing air and sound to pass through the ear tip. In embodiments of the present invention, the acoustic vent has a predetermined acoustic mass. In embodiments of the present invention, the predetermined acoustic mass of the ear tip is less than 2000 kilograms per meter⁴ (meter to the fourth power). In embodiments of the present invention, the transmit coil includes a coil winding wound around the ferromagnetic material.

In one embodiment, the present invention is directed to a method of acoustically connecting a proximal end of an ear tip to a distal end of an ear tip wherein the ear tip includes a transmit coil wrapped around a core, the core having an central channel extending from a proximal end of the core to a distal end of the core, and the ear tip having a passage extending from a proximal end of the ear tip to a distal end of the ear tip, the method including the steps of: passing an electrical current through the transmit coil; passing acoustic signals through the central channel; and passing acoustic signals through the passage. In embodiments of the present invention, the acoustic signals comprise sound. In embodiments of the present invention, sound and air pass through the passage. In embodiments of the present invention, a proximal end of the central channel connects to the passage at a point within the ear tip. In embodiments of the present invention, a distal end of the central channel is connected to a first opening in the distal end of the ear tip and the distal end of the passage is connected to a second opening in the distal end of the ear tip.

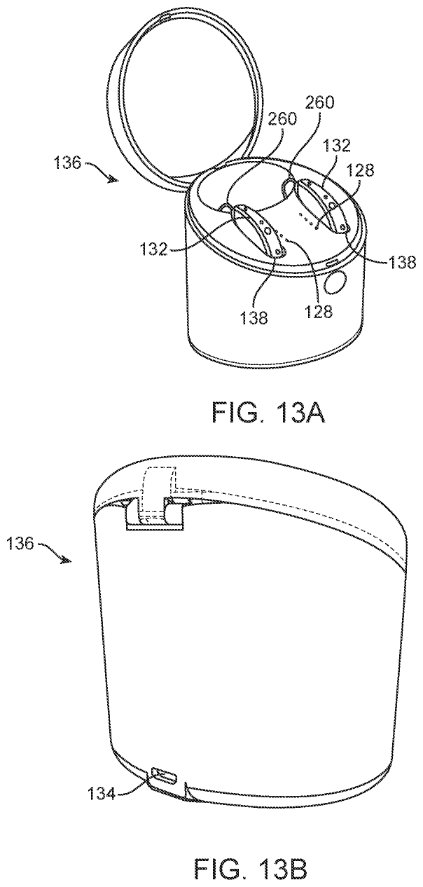

FIG. 13A is a top perspective view of a charging station 136 for use in charging audio processors 132. FIG. 13B is a back perspective view of a charging station 136, including AC adapter port 134 for use in charging audio processors 132. In FIGS. 13A and 13B, audio processors 132 may be positioned in charging slots 138. Charging status LEDs 128 may be used to communicate the charge status of audio processors 132 positioned in charging slots 138.

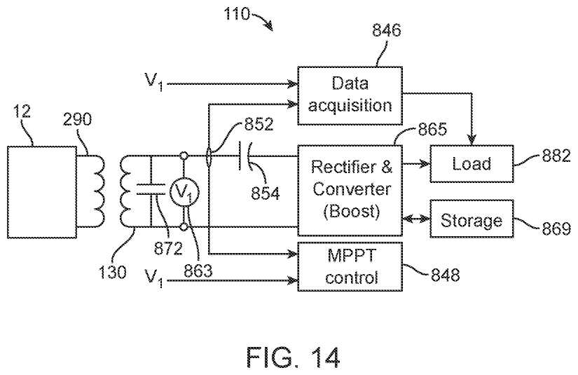

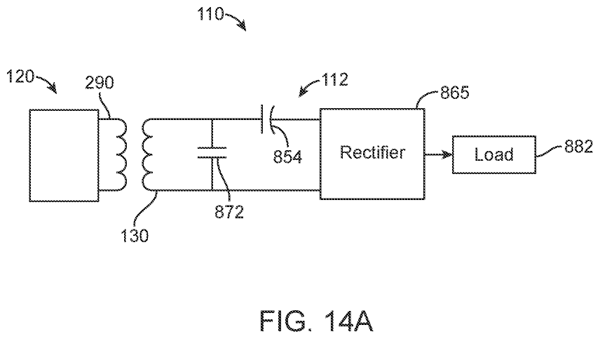

FIG. 14 is a block diagram of an inductively coupled contact hearing device 112 and ear tip 120 according to the present invention. In embodiments of the invention, contact hearing device 112 may also be referred to as a medial ear canal assembly. In FIG. 14, the output of ear tip 120 may be inductively coupled through transmit coil 290 to receive coil 130 on contact hearing device 112. In embodiments of the invention, ear tip 120 may be referred to as a lateral ear canal assembly. In embodiments of the invention, inductive coupling may induce a current in receive coil 130 on contact hearing device 112. In embodiments of the invention, the inductively induced current may be measured by current sensor 852. In embodiments of the invention, inductive coupling may induce an output voltage V₁ across receive coil 130. In embodiments of the invention, the induced output voltage may be measured by a voltage meter 863. In embodiments of the invention, the measured current and voltage may be used by MPPT control 848 and data acquisition circuit 846. In embodiments of the invention, the output of receive coil 130 may be further connected to a rectifier and converter circuit 865 through capacitor 854. In embodiments of the invention, receive coil 130 may be connected directly to rectifier and converter circuit 865 (eliminating capacitor 854). In embodiments of the invention, receive coil 130 may be connected to a rectifier circuit. In FIG. 14, capacitor 854 may be positioned between the output of receive coil 130, which may include capacitor 872, and the input of rectifier and converter circuit 865. The output of rectifier and converter circuit 865 may be connected to load 882 and to storage device 869. In embodiments of the invention, rectifier and converter circuitry 865 may include circuitry which provides power to storage device 869 and transmits power from storage device 869 to load 882 when required. In embodiments of the invention, storage device 869 may be connected directly to receive coil 130 or to other circuitry adapted to harvest energy from receive coil 130 and deliver energy to load 882. Load 882 may be, for example, a microactuator positioned on the contact hearing device 112 such that load 882 vibrates the tympanic membrane of a user when stimulated by signals received by receive coil 130. Storage device 869 may be, for example, a rechargeable battery.

In embodiments of the invention, transmit coil 290 may comprise a transmit coil, such as, for example, transmit coil 290 and coil 130 may comprise a receive coil, such as, for example, receive coil 130. In embodiments of the invention, transmit coil 290 and receive coil 130 may be elongated coils manufactured from a conductive material. In embodiments of the invention, transmit coil 290 and receive coil 130 may be stacked coils. In embodiments of the invention, transmit coil 290 and receive coil 130 may be wound inductors. In embodiments of the invention, transmit coil 290 and receive coil 130 may be wound around a central core. In embodiments of the invention, transmit coil 290 and receive coil 130 may be wound around a core comprising air. In embodiments of the invention, transmit coil 290 and receive coil 130 may be wound around a magnetic core. In embodiments of the invention, transmit coil 290 and receive coil 130 may have a substantially fixed diameter along the length of the wound coil.

In embodiments of the invention, rectifier and converter circuit 865 may comprise power control circuitry. In embodiments of the invention, rectifier and converter circuit 865 may comprise a rectifier. In embodiments of the invention, rectifier and converter 865 may be a rectifying circuit, including, for example, a diode circuit, a half wave rectifier or a full wave rectifier. In embodiments of the invention, rectifier and converter circuit 865 may comprise a diode circuit and capacitor. In embodiments of the invention, energy storage device 869 may be a capacitor, a rechargeable battery or any other electronic element or device which is adapted to store electrical energy.

In FIG. 14, the output of MPPT control circuit 848 may control rectifier and converter circuit 865. Rectifier and converter circuit 865 may supply energy to and receive energy from storage device 869, which may be, for example, a rechargeable battery. Data acquisition circuit 846 and rectifier and converter circuit 865 may be used to drive load 882, with data acquisition circuit 846 proving load 882 with control data (e.g. sound wave information) and rectifier and converter circuit 865 providing load 882 with power. In embodiments of the invention, rectifier and converter circuit 865 may be used to drive load 882 directly, without information from a data circuit such as data acquisition circuit 846. In embodiments of the invention, rectifier and converter circuit 865 may be used to drive load 882 directly without energy from storage device 869. The power provided by rectifier and converter circuit 865 may be used to drive load 882 in accordance with the control data from data acquisition circuit 846. Load 882 may, in some embodiments of the invention, be a transducer assembly, such as, for example, a balanced armature transducer.

In embodiments of the invention, information and/or power may be transmitted from ear tip 120 to contact hearing device 112 by magnetically coupling transmit coil 290 to receive coil 130. When the coils are inductively coupled, the magnetic flux generated by transmit coil 290 may be used to generate an electrical current in receive coil 130. When the coils are inductively coupled, the magnetic flux generated by transmit coil 290 may be used to generate an electrical voltage across receive coil 130. In embodiments of the invention, the signal used to excite transmit coil 290 on ear tip 120 may be a push/pull signal. In embodiments of the invention, the signal used to excite transmit coil 290 may have a zero crossing. In embodiments of the invention, the magnetic flux generated by transmit coil 290 travels through a pathway that includes a direct air pathway that is not obstructed by bodily components. In embodiments of the invention, the direct air pathway is through air in the ear canal of a user. In embodiments of the invention, the direct air pathway is line of sight between ear tip 120 and contact hearing device 112 such that contact hearing device 112 is optically visible from ear tip 120.

In embodiments of the invention, the output signal generated at receive coil 130 may be rectified by, for example, rectifier and converter circuit 865. In embodiments of the invention, a rectified signal may be used to drive a load, such as load 882 positioned on contact hearing device 112. In embodiments of the invention, the output signal generated at receive coil 130 may contain an information/data portion which includes information transmitted to contact hearing device 112 by transmit coil 290. In embodiments of the invention, at least a portion of the output signal generated at receive coil 130 may contain energy or power which may be scavenged by circuits on contact hearing device 112 to charge, for example, storage device 869.

FIG. 14A is a block diagram of an inductively coupled contact hearing system according to the present invention. In FIG. 14A, contact hearing system 110 includes Ear Tip 120 (which may also be referred to as a Mag Tip) and contact hearing device 112. Ear Tip 120 may include a transmit coil 290. Contact hearing device 112 may include receive coil 130, parasitic capacitance 872, capacitor 854, rectifier and converter circuit 865 and load 882.

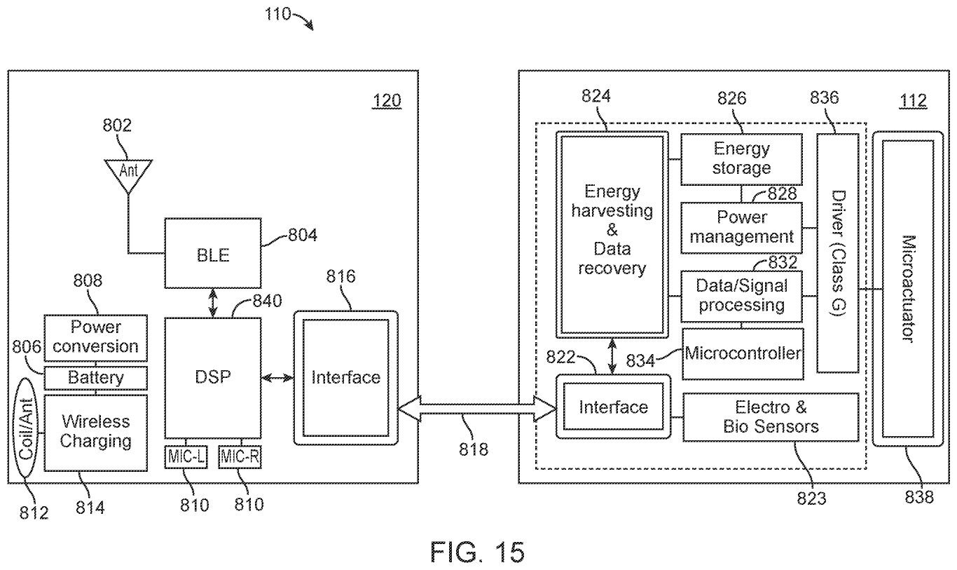

FIG. 15 is a block diagram of a contact hearing system 110, including an ear tip 120 (which may also be referred to as a processor) and contact hearing device 112 according to the present invention. In FIG. 15, ear tip 120 may include an external antenna 802 adapted to send and receive signals from an external source such as a cell phone (see FIG. 2). External antenna 802 may be connected to a circuit for processing signals received from external antenna 802, such as blue tooth circuit 804, which, in some embodiments, may be a blue tooth low energy circuit. The output of Bluetooth circuit 804 may be connected to digital signal processor 840, which may also include inputs from microphones 810. Ear canal assembly 12 may further include battery 806 and power conversion circuit 808 along with charging antenna 812 (which may be a coil) and wireless charging circuit 814. Digital signal processor 840 may be connected to interface circuit 816, which may be used to transmit data and power from ear tip 120 to contact hearing device 112. In embodiments of the invention, power and data may be transmitted between ear tip 120 and contact hearing device 112 over power/data link 818 by inductive coupling to provide transmission of the data and power. Alternatively, separate modes of transmission may be used for the power and data signals, such as, for example, transmitting the power using radio frequency or light and the data using inductive coupling.

In FIG. 15, power and data transmitted to contact hearing device 112 may be received by interface circuit 822. Interface circuit 822 may be connected to energy harvesting and data recovery circuit 824 and to electrical and biological sensors 823. In FIG. 2, contact hearing device 112 may further include energy storage circuitry 826, power management circuitry 828, data and signal processing circuitry 832, and microcontroller 834. Contact hearing device 112 may further include a driver circuit 836 and a microactuator 838. In the illustrated embodiment, data transmitted from contact hearing device 112 may be received by interface circuit 816 on ear tip 120.

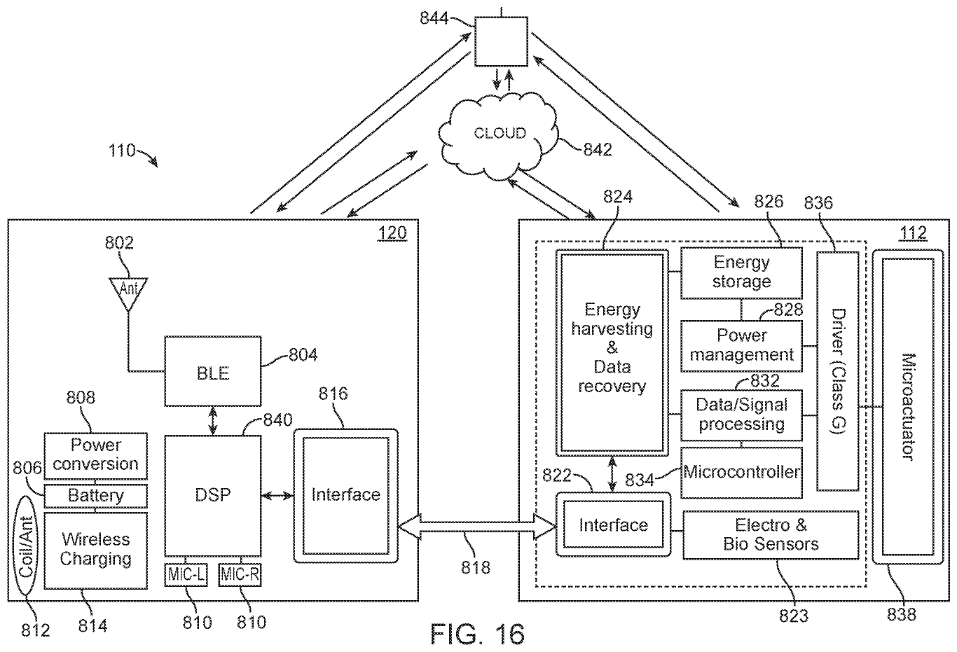

FIG. 16 is a block diagram of a contact hearing system 110, adapted for communication with external devices according to the present invention. In FIG. 3, contact hearing system 110 is adapted to communicate with external devices such as cell phone 844 or cloud computing services 842. Such communication may occur through, for example, external antenna 802 on ear tip 120 or, in some embodiments directly from contact hearing device 112.

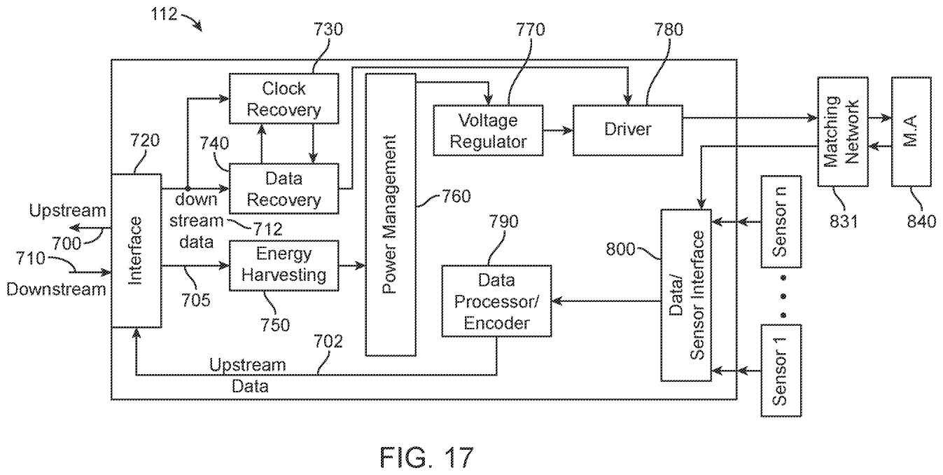

FIG. 17 is a block diagram of a contact hearing device 112 according to an embodiment of the present invention. In FIG. 17, contact hearing device 112 includes interface 720, clock recovery circuit 730, data recovery circuit 740 and energy harvesting circuit 750. In embodiments of the invention, interface 720 is adapted to transmit data from contact hearing device 112 and to receive data transmitted to contact hearing device 112. Interface 720 may be an inductive interface. Contact hearing device 112 may further include power management circuit 760, voltage regulator 770, driver 780, data processor encoder 790 and data/sensor interface 800.

In FIG. 17, upstream data 702 collected from data processor/encoder 790 may be transmitted via interface 720 as a part of upstream signal 700. Downstream signal 710 may be transmitted to interface 720, which may extract the data portion and may distribute downstream data 712 to data recovery circuit 740 and clock recovery circuit 730. Interface 720 may further transmit at least a portion of downstream signal 710 to energy harvesting circuit 750. The output of energy harvesting circuit 750 may be transmitted to power management circuit 760, which may then distribute energy to voltage regulator 770. Voltage regulator 770 may distribute its output to driver 780, which may also receive input from data recovery circuit 740. The output of driver 780 may be sent through matching network 831 to drive, for example, microactuator 840.

Microactuator 840 may include sensors (not shown) which generate data about the function of microactuator 840. This data may be transmitted back to contact hearing device 112 through matching network 831 and to data/sensor interface 800, which, in turn may transmit the sensor information to data processor/encoder 790, which generates upstream data 702. Data/sensor interface 800 may also receive information from other sensors (e.g. Sensor 1 to Sensor n in FIG. 4), which data is, in turn, transmitted to data processor/encoder 790 and becomes part of upstream data 702.

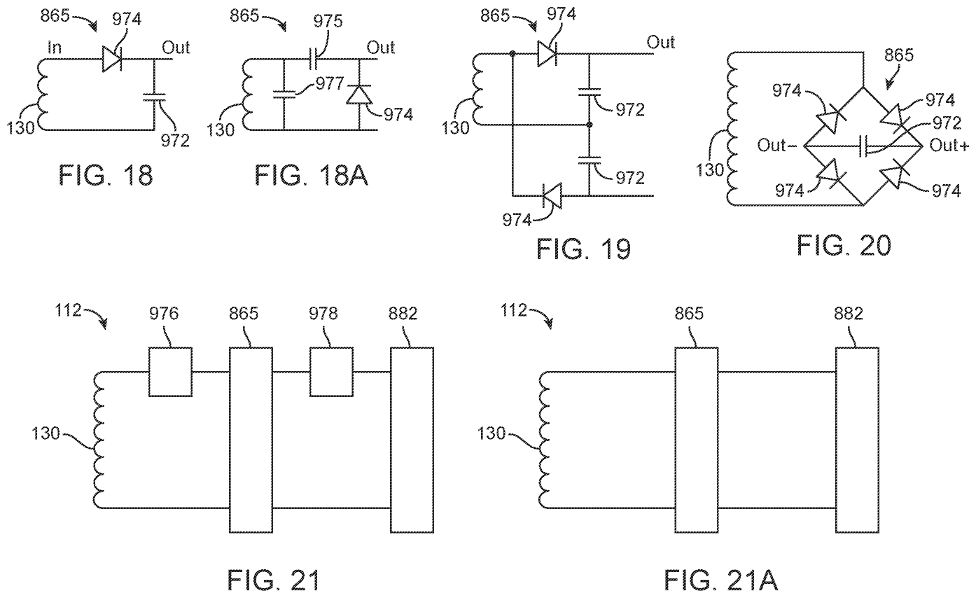

FIG. 18 is a diagram of a rectifier and converter circuit 865 according to the present invention. In FIG. 18, rectifier and converter circuit 865 may include diode 974 and capacitor 972. In embodiments of the invention, the input to rectifier and converter circuit 865 may be connected directly to receive coil 130. In embodiments of the invention, the output of rectifier and converter circuit 865 may be coupled directly to a load, such as, for example, a transducer or a balanced armature transducer. In embodiments of the invention, the output of rectifier and converter circuit 865 may be coupled to the windings in a load, such as, for example, a transducer or a balanced armature transducer.

FIG. 18A is a diagram of a rectifier and converter circuit 865 according to the present invention. In embodiments of the invention, rectifier and converter circuit 865 may comprise a Villard Circuit. In embodiments of the invention, rectifier and converter circuit 865 may be a voltage multiplier circuit. In FIG. 18, rectifier and converter circuit 865 may include diode 974, AC filter capacitor 975 (which may be a series capacitor) and resonant capacitor 977. In embodiments of the invention, the input to rectifier and converter circuit 865 may be connected directly to receive coil 130. In embodiments of the invention, the output of rectifier and converter circuit 865 may be coupled directly to a load, such as, for example, a transducer or a balanced armature transducer. In embodiments of the invention, the output of rectifier and converter circuit 865 may be coupled to the windings in a load, such as, for example, a transducer or a balanced armature transducer.

FIG. 19 is a diagram of an alternative rectifier and converter circuit 865 according to the present invention. In embodiments of the invention, rectifier and converter circuit 865 may include diodes 974 and capacitors 972 which may form, for example bridge circuits such as, for example a half wave bridge.

FIG. 20 is a diagram of an alternative rectifier and converter circuit according to the present invention. In embodiments of the invention, rectifier and converter circuit 865 may include diodes 974 and capacitors 972 which may form, for example bridge circuits such as, for example, a full wave bridges. In embodiments of the invention, rectifier and converter circuit 865 may be connected to receive coil 130.

FIG. 21 is a diagram of a portion of a contact hearing device 112 according to the present invention. In embodiments of the invention, the input to rectifier and converter circuit 865 may be connected to receive coil 130 through additional circuitry, such as, for example, capacitor 854 or input circuitry 976. In embodiments of the invention, the output of rectifier and converter circuit 865 may be coupled to a load, such as, for example, a transducer or a balanced armature transducer through an output circuit 978. In embodiments of the invention, output circuit 978 may be, for example, a capacitor, an inductor, a combination of electrical or electronic components and/or a matching circuit.

FIG. 21A is a diagram of a portion of a contact hearing device according to the present invention. In FIG. 21A, contact hearing device 112 may include receive coil 130, connected to rectifier and converter circuit 865, which, in turn, may be connected to load 882, which may be, for example, a microactuator, for example a balanced armature microactuator.

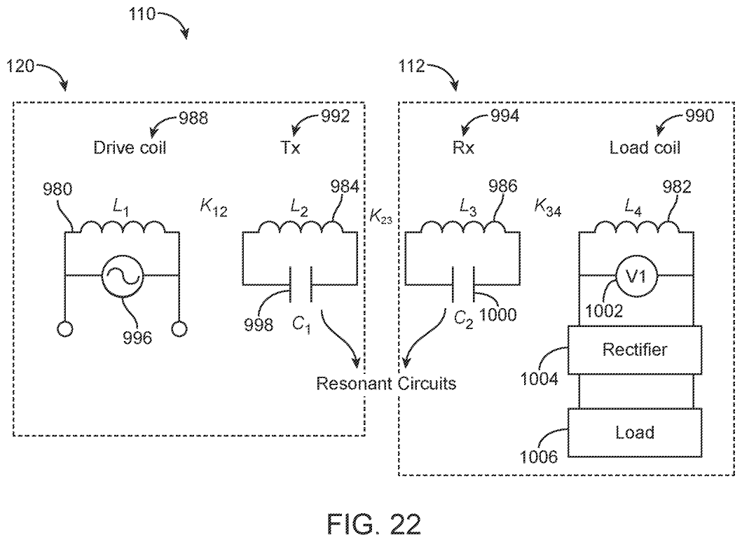

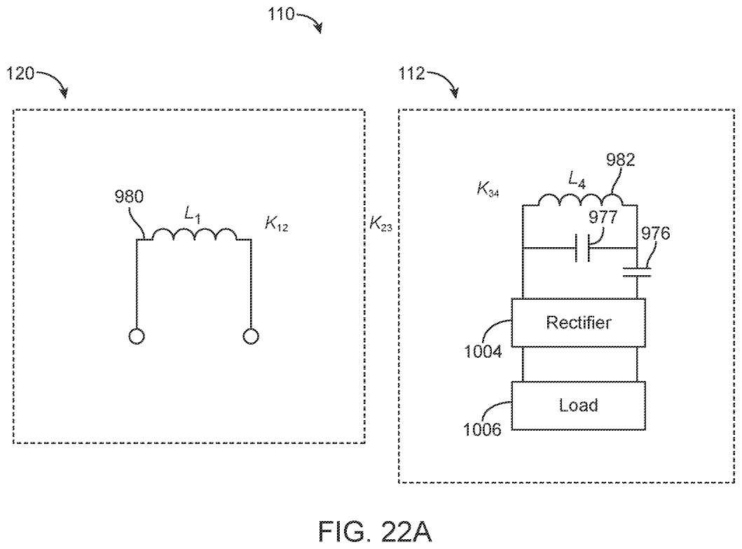

FIG. 22 is a circuit diagram of transmitter and receiver components of a contact hearing system 110 according to embodiments of the present invention. In embodiments of the invention, ear tip 120 may include a drive circuit 988, which may also be referred to as a transmit circuit. Drive circuit 988 may include coil L1 980 and signal source 996. In embodiments of the invention, ear tip 120 may further include transmit resonant circuit 992. In embodiments of the invention, transmit resonant circuit 992 may include resonant transmit coil L2 994 and resonant transmit capacitor C1 998. In embodiments of the invention, contact hearing device 112 may include load circuit 990. In embodiments of the invention, load circuit 990 may include load coil 982, voltage detector 1002, rectifier 1004 and load 1006. In embodiments of the invention, contact hearing device 112 may include receive resonant circuit 994. In embodiments of the invention, receive resonant circuit 994 may include resonant receive coil 986 and resonant receive capacitor C2 1000.

FIG. 22A is a circuit diagram of transmitter and receiver components of a contact hearing system according to embodiments of the present invention. In the contact hearing system 110 of FIG. 22A, ear tip 120 includes drive coil L1 980. In the contact hearing system 110 of FIG. 22A, contact hearing device 112 includes load coil L4 982, resonance capacitor 977 (which may also be referred to as a tuning capacitor), AC filter capacitor 975, rectifier circuit 1004 and load 1006.

In embodiments of the invention, drive coil 980 may be a transmit coil such as, for example, transmit coil 290. In embodiments of the invention, load coil 982 may be a receive coil such as, for example, receive coil 130. In embodiments of the invention, rectifier 1004 may be a rectifier and converter circuit such as, for example, rectifier and converter circuit 865. In embodiments of the invention, load 1006 may be an actuator, such as, for example microactuator 140. In embodiments of the invention, microactuator 140 may be, for example, a balanced armature microactuator.

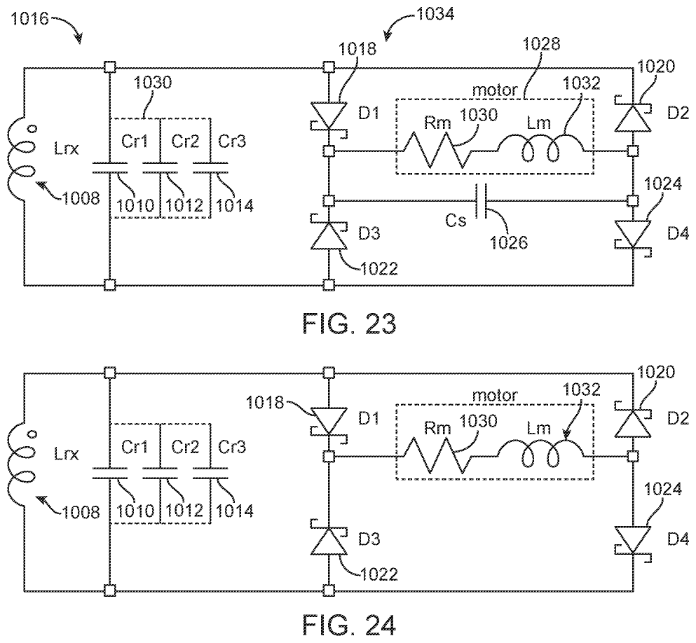

FIGS. 23 and 24 are circuit diagrams of components of a receiver 1016 for use in a contact hearing system 110 according to the present invention. In embodiments of the invention, receiver 1016 may be constructed in a full-wave rectifier receiver configuration, including a smoothing capacitor. In embodiments of the invention, receiver 102 includes receive inductor Lrx 1008, receive capacitor array 1030, diode bridge 1032, motor 1028, and smoothing capacitor 1026. In embodiments of the invention, receive capacitor array 1030 may include one or more receive capacitors, such as, receive capacitor Cr1 1010, receive capacitor Cr2 1012 and receive capacitor Cr3 1014. In embodiments of the invention, diode bridge 1034 may include one or more diodes, such as, diode D1 1018, diode D2 1020, diode D3 1022, and diode D4 1024. In embodiments of the invention, diode bridge 1034 may be arranged as a full wave rectifier bridge with a load, such as, for example, motor 1028 connected across the output of the full wave rectifier. In embodiments of the invention (such as the one illustrated in FIG. 23), a smoothing capacitor Cs 1026 may be connected across the output of the full wave rectifier in parallel with the motor 1028. In embodiments of the invention (such as the one illustrated in FIG. 24), the smoothing capacitor may be omitted. In embodiments of the invention, the diodes used in diode bridge 1034 may be Schottky diodes. In embodiments of the invention, the electrical characteristics of motor 1028 may be represented by the series circuit which includes motor resistor 1030, representing the resistance of the circuitry in motor 1028 and motor inductor 1032, representing the inductance of motor 1028 at the frequency of operation.

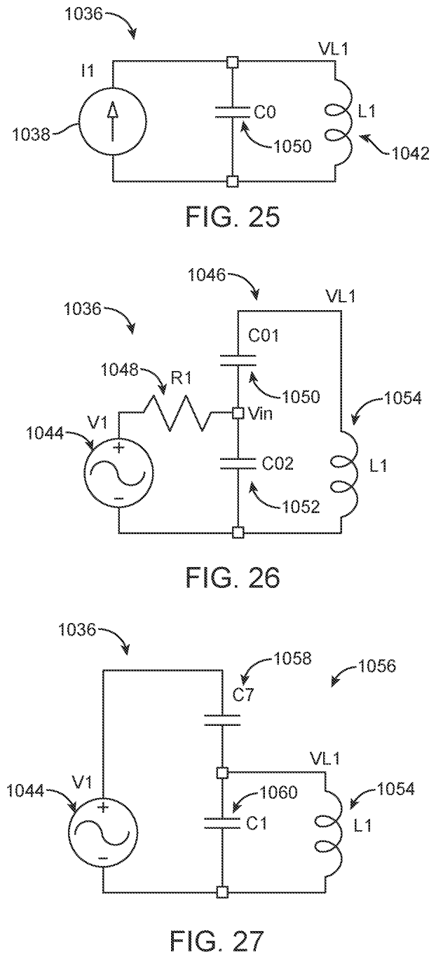

FIG. 25 is a circuit diagram of components of a transmitter 1036 for use in a contact hearing system 110 according to the present invention. In embodiments of the invention, transmitter 1036 may be a current source 1038 connected in parallel with one or more output capacitors, such as C0 1040 and output coil L1 1042. In the embodiment of the invention, illustrated in FIG. 25, the transmitter may be a parallel drive with the signal input modeled as current source 1038. The configuration illustrated in FIG. 25 is advantageous because it requires a low input current.

FIG. 26 is a circuit diagram of components of a transmitter 1036 for use in a contact hearing system 110 according to the present invention. In embodiments of the invention, transmitter 1036 may be modeled as a voltage source 1044 feeding a capacitive transformer/divider 1046 through a resistor R1 1048. In this embodiment, capacitive transformer/divider 1046 may be modeled as Capacitor CO1 1050 in series with capacitor CO2 1052, which are in parallel with inductor L1 1054. The embodiment of the transmitter, illustrated in FIG. 26 is advantageous because it may be used to generate a large VL1 when V1 is small, thus allowing the circuit to be driven by, for example, a battery having a limited output voltage, for example, an output voltage in the range of 3 Volts. In this embodiment, voltage source V1 1044, in parallel with resistor R1 1048, combine to form a quasi-current source. In the embodiment illustrated, the resonant frequency will be a function of the series combination of capacitor CO1 1050, capacitor CO2 1052 and Inductor L1 1054.

FIG. 27 is a circuit diagram of components of a transmitter 1036 for use in a contact hearing system 110 according to the present invention. In embodiments of the invention, the circuit illustrated may represent a parallel drive arrangement for transmitter 1036. In embodiments of the invention, transmitter 1036 may be modeled as a voltage source V1 1044 feeding a parallel drive circuit 1056. In embodiments of the invention, parallel drive circuit 1056 may include capacitor C7 1058, capacitor C1 1060 and inductor L1 1054. In embodiments of the invention, capacitor C7 1058 adds impedance to voltage source V1 1044 to create a quasi-current source. In embodiments of the invention, C7 may be small compared to C1 1060 in order to ensure that most of the tank current flows in the L1-C1 loop, rather than in the L1-C7 loop. In embodiments of the invention, the resonant frequency will depend on the series combination of inductor L1 1054 with the parallel combination of capacitor C1 1060 and capacitor C7 1058.

In embodiments of the invention, using inductive coupling for power and/or data transfer in a contact hearing system may result in benefits over other methods of power and/or data transfer, including: reduced sensitivity to directionality; reduced sensitivity to motion in components of the contact hearing system; improved patient comfort; reduced sensitivity to the presence of bodily fluids, including cerumen; reduced sensitivity to the presence of tissue between the ear tip and the contact hearing device; reduced sensitivity to tissue loading; reduced sensitivity to the distance between the ear tip and the contact hearing device. In embodiments of the invention, power and data transfer may be separated (e.g. different frequencies) or combined.

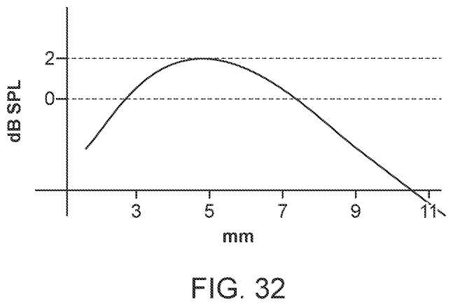

In embodiments of the invention, data and power may be transferred from an ear tip to a contact hearing device using near field magnetic coupling. In embodiments of the invention, data may be transferred from an ear tip to a contact hearing device using near field magnetic coupling. In embodiments of the invention, power may be transferred from an ear tip to a contact hearing device using near field magnetic coupling. In embodiments of the invention, the use of near field magnetic coupling results in a power transfer wherein the power output from the contact hearing device remains relatively constant even when the distance between the ear tip and the contact hearing device varies. In embodiments of the invention, as illustrated in FIG. 32, the use of near field magnetic coupling results in a power output wherein the output of the contact hearing device varies by less than 2 dB SPL when the distance between the ear tip and the contact hearing device varies between 3 and 7 millimeters. In embodiments of the invention, as illustrated in FIG. 32, the use of near field magnetic coupling results in a power output wherein the output of the contact hearing device varies by less than 2 dB SPL when the distance between the ear tip and the contact hearing device is approximately 3 millimeters. In embodiments of the invention, data and power may be transmitted from an ear tip to a contact hearing device using resonant inductive coupling. In embodiments of the invention, the receive coil and the transmit coil may be connected through resonant inductive coupling. In embodiments of the invention, data and power may be transmitted from an ear tip to a contact hearing device using near field magnetic induction. In embodiments of the invention, data and power may be transmitted from an ear tip to a contact hearing device using a near field magnetic induction link.

In embodiments of the invention, such near field magnetic coupling could also be used to remotely power and/or deliver signal to neuro-stim implants. In embodiments of the invention the actuator may be replaced by electrodes. In embodiments of the invention, such near field magnetic coupling could also be used to remotely power in-body valves for, for example, bladder control.

In embodiments of the invention, the separation between the transmit coil and the receive coil may be between approximately five and nine millimeters when the system is placed in a user's ear.

In one embodiment, the present invention is directed to a method of transmitting information from an ear tip to a contact hearing device, the method including the steps of: exciting a transmit coil, the transmit coil being positioned in the ear tip, wherein the transmit coil is wound on a core, the core including a ferromagnetic material; radiating an electromagnetic field from the first coil through the ear canal of a user; receiving the radiated electromagnetic field at a receive coil, the receive coil being positioned on a contact hearing device, the contact hearing device including a receive coil without a ferrite core; and transmitting the information from the transmit coil to the receive coil using near-field radiation. In embodiments of the invention, the ear tip includes the transmit coil and the contact hearing device includes the receive coil. In embodiments of the invention, the method includes the step of adapting the ear tip such that it positions the medial end of the transmit coil to be within between 3 and 7 millimeters of the lateral end of the receive coil when the ear tip and contact hearing device are positioned in the ear canal of a user. In embodiments of the invention, the method includes the step of adapting the ear tip such that when it is positioned in the ear canal of a user more than fifty percent of magnetic flux lines emanating from the transmit coil couple through the receive coil. In embodiments of the invention, the method includes the step of adapting the ear tip such that when it is positioned in the ear canal of a user more than seventy five percent of a magnetic field generated by the transmit coil is coupled to the receive coil. In embodiments of the invention, the method includes the step of generating a signal in the transmit coil induces current in the receive coil, wherein the induce current is induced by the presence of a magnetic field generated at the transmit coil. In embodiments of the invention, the current induced is proportional to the magnetic field at the transmit coil. In embodiments of the invention, the step of generating a signal in the transmit coil results in a voltage generated across the receive coil wherein the generated voltage is a product of the magnetic field generated at the transmit coil. In embodiments of the invention, the voltage generated is proportional to the magnetic field at the transmit coil. In embodiments of the invention, the transmitted information is transmitted in an amplitude modulated (AM) signal. In embodiments of the invention, the transmitted information is demodulated by a demodulator attached to a receive coil. In embodiments of the invention, the transmit coil is magnetically coupled to the receive coil. In embodiments of the invention, the coupling between the transmit and receive coils is between approximately 0.1 percent and approximately 3.0 percent. In embodiments of the invention, information and power are transmitted from the transmit coil to the receive coil through the interaction of magnetic fields generated in the transmit coil with the receive coil. In embodiments of the invention, the core includes a ferrite material.

In one embodiment, the present invention is directed to a method of transmitting information from an ear tip to a contact hearing device, the method including the steps of: exciting a transmit coil, the transmit coil being positioned in an ear tip, wherein the transmit coil is wound on a ferrite core; radiating an electromagnetic field from the first coil through the ear canal of a user; receiving the radiated electromagnetic field at a receive coil, the receive coil being positioned on a contact hearing device without a ferrite core; and transmitting the information from the transmit coil to the receive coil using a near-field radiation. In embodiments of the invention, the first and second coils are inductively coupled. In embodiments of the invention, inductive coupling is used to link the first coil to the second coil. In embodiments of the invention, the information is transmitted from the first coil to the second coil using near-field magnetic coupling. In embodiments of the invention, the information is transmitted from the first coil to the second coil using resonant inductive coupling. In embodiments of the invention, the information is transmitted from the first coil to the second coil using near-field magnetic induction. In embodiments of the invention, the information is transmitted from the first coil to the second coil using a near-field magnetic induction link. In embodiments of the invention, the output of the contact hearing device varies by less than two decibels sound pressure level (dB SPL) when the distance between the transmit and receive coils varies by between three and seven millimeters. In embodiments of the invention, the receive coil is a part of a receive coil assembly, the receive coil assembly including: the receive coil; at least one disk positioned at a distal end of the receive coil, the at least one disk including a ferromagnetic material. In embodiments of the invention, the receive coil is wound with a central core of a non-ferromagnetic material. In embodiments of the invention, the non-ferromagnetic material is, at least in part, air. In embodiments of the invention, the outer diameter of the at least one disk is substantially the same as the outer diameter of the receive coil. In embodiments of the invention, the at least one disk includes a hole therethrough. In embodiments of the invention, the at least one disk is two disks. In embodiments of the invention, a printed circuit board including electronic components is affixed to a side of the at least one disk opposite the side to which the receive coil is affixed. In embodiments of the invention, the at least one disk includes a ferrite material.

In one embodiment the present invention is directed to a method of transmitting information from an ear tip to a contact hearing device, the method including the steps of exciting a transmit coil, the transmit coil being positioned in an ear tip, wherein the transmit coil is wound on a ferromagnetic core; radiating an electromagnetic field from the transmit coil through an ear canal of a user; receiving the radiated electromagnetic field at a receive coil, the receive coil being positioned on a contact hearing device, the receive coil having a core of a non-ferromagnetic material; and transmitting the information from the transmit coil to the receive coil using the electromagnetic field. In embodiments of the invention, the transmit and receive coils are inductively coupled. In embodiments of the invention, inductive coupling is used to link the transmit coil to the receive coil. In embodiments of the invention, the information is transmitted from the transmit coil to the receive coil using near-field magnetic coupling. In embodiments of the invention, the information is transmitted from the transmit coil to the receive coil using resonant inductive coupling. In embodiments of the invention, information is transmitted from the transmit coil to the receive coil using near-field magnetic induction. In embodiments of the invention, information is transmitted from the transmit coil to the receive coil using a near-field magnetic induction link. In embodiments of the invention, the output of the contact hearing device varies by less than two decibels sound pressure level (dB SPL) when the distance between the transmit and receive coils varies by between three and seven millimeters. In embodiments of the invention, the receive coil is a part of a receive coil assembly, the receive coil assembly including: the receive coil; at least one disk positioned at a distal end of the receive coil, the at least one disk including a ferromagnetic material. In embodiments of the invention, the receive coil is wound with a central core of a non-ferromagnetic material. In embodiments of the invention, the non-ferromagnetic material is, at least in part, air. In embodiments of the invention, an outer diameter of the at least one disk is substantially the same as an outer diameter of the receive coil. In embodiments of the invention, the at least one disk includes a hole therethrough. In embodiments of the invention, the at least one disk is two disks. In embodiments of the invention, a printed circuit board including electronic components is affixed to a side of the at least one disk opposite a side to which the receive coil is affixed. In embodiments of the invention, the electronic components on the printed circuit board includes a demodulation circuit. In embodiments of the invention, the demodulation circuit is a diode demodulator. In embodiments of the invention, the at least one disk includes a ferrite material.

In embodiments of the invention, the transmit coil may include a coil with an air core. In embodiments of the invention, the transmit coil may include a coil wound around a ferrite core. In embodiments of the invention, the transmit coil may include a coil wound around a ferrite core with a channel through the center of the ferrite core, the channel forming an opening from the proximal end to the distal end of the ferrite core. The channel may further be positioned and sized to form an acoustic vent, allowing sound to pass through the ferrite core. In embodiments of the invention, the receive coil may include a coil wound around an air core. In embodiments of the invention, the receive coil may include a coil wound around ferrite core.

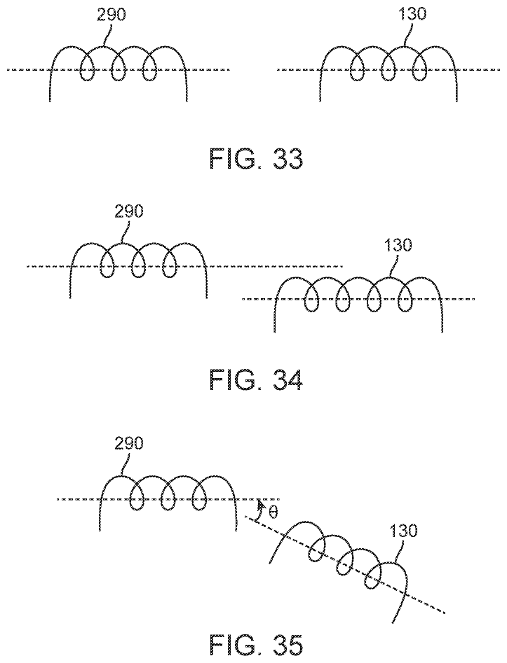

As illustrated in FIGS. 33-35, in embodiments of the invention, the central axis of the receive core and the central axis of the transmit core may be substantially parallel when the ear tip and the contact hearing device are positioned in the ear canal of a user. In embodiments of the invention, the central axis of the receive core and the central axis of the transmit core form an angle of not greater than 15 degrees. In embodiments of the invention, the central axis of the receive core and the central axis of the transmit core form an angle of not more than approximately 25 degrees. In embodiments of the invention, the system has a signal reduction of less than 0.5 dB over an angle of between plus and minus 20 degrees from full alignment.

In embodiments of the invention, a reduction in output (in dB) for a receive coil assembly as a function of the transmit to receive coil angle as a function of the distance L, and the angles θ₁, θ₂ and θ₃ over a range of ±45°. In embodiments of the invention, the angle θ may be greater than ±450 and distance between the transmit coil and the receive coil may be between 2 and 12 mm.

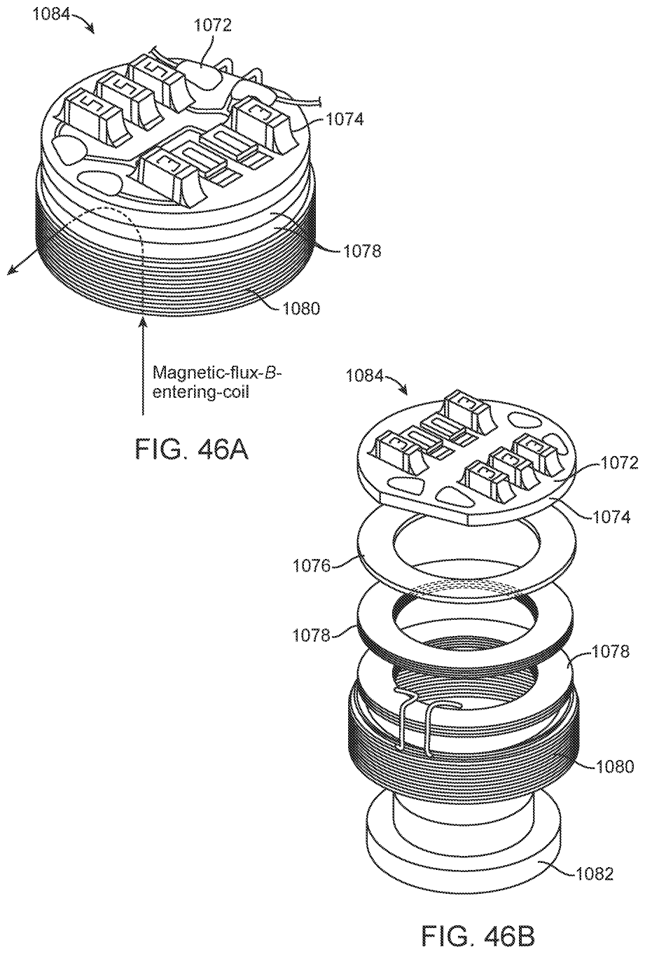

As illustrated in FIGS. 44, 45, 46A and 46B, a receive circuit assembly 1084 may include receive circuit board 1074, which may have mounted thereon receive circuit components 1072. Receive circuit assembly may further include receive coil winding (Rx Coil) 1080 and ferrite disk(s) 1078. Ferrite disc(s) 1078 may be attached to receive circuit board 1074 by adhesive 1076. Receive coil winding 1080 may include a plug 1082 at a proximal end thereof. In embodiments of the invention, receive coil winding 1080 may be wound around a core of a non-ferromagnetic material, such as, for example air. In embodiments of the invention, ferrite disk(s) 1078 may include a hole in the center of the disks. In embodiments of the invention, the hole in the center of ferrite disk(s) 1078 may be substantially the same diameter as the core of receive coil winding 1080. FIG. 46A illustrates the flux path through receive circuit assembly 1084 wherein the flux may be generated by an ear tip which is located a distance away from receive circuit assembly 1084 in the ear canal of a user. In embodiments of the invention, the magnetic flux may be generated in a coil positioned in the ear tip and may be a signal representative of information (e.g. audio information) to be transmitted to a contact hearing device which includes receive circuit assembly 1084. In FIG. 46A flux enters receive circuit assembly 1084 at a proximal end thereof and passes through receive coil winding 1080 and then through ferrite disk(s) 1078. In FIG. 46A the flux passing through receive circuit assembly 1084 induces a current in receive coil winding 1080. In embodiments of the invention, the current induced in receive coil winding 1080 will be conducted electrical components on contact hearing device 112, which will demodulate the received signal and transmit that signal to a microactuator 140 which may be in contact with the tympanic membrane of a user.

In embodiments of the invention, receive circuit assembly 1084 includes receive coil windings 1080 which may be backed by one or more (e.g. two) two ring-shaped ferrite layers (which may also comprise or be referred to as ferrite disk(s)) 1078 to which receive circuit components (e.g. one of the demodulator circuit described herein) are attached. In embodiments of the invention, the ferrite layers may increase the strength of the received signals in multiple ways.

In embodiments of the invention, the ferrite layers may increase the inductance and Q of receive circuit assembly 1084. In embodiments of the invention, the ferrite layers may shunt magnetic flux entering receive coil windings 1080 to the outside of receive coil windings 1080 on the distal (PCB) end of receive coil windings 1080. In embodiments of the invention, magnetic flux may be shunted because the ferrite layers have high permeability and low reluctance compared to air and PCB material. In embodiments of the invention, this shunting of the magnetic flux results in the magnetic field being coupled more tightly around the receive coil windings 1080, which increases inductance without significant effect on the AC resistance. The Q increases directly from its defining equation Q=2πfL/R_{AC}, where f is the carrier frequency and L and R_{AC} are the inductance and resistance at the carrier frequency, respectively.

In embodiments of the invention, shunting the field, the ferrite layers also shield receive circuit board 1074 and receive circuit components 1072 from the magnetic field and reduce loading of the magnetic field by eddy currents in the metal traces of receive circuit board 1075. As a result, the field inside receive coil windings 1080 is stronger, compared to a receive circuit assembly 1084 which did not include any ferrite layers (e.g. ferrite disk(s) 1078 and, therefore, may produce a higher signal strength at the output of receive circuit assembly 1084.

In embodiments of the invention, by acting as spacers to separate receive circuit board 1074 from a distal end of receive coil windings 1080 decreases magnetic-field loading caused by the presence of receive circuit board 1074 and receive circuit components 1072 at the distal end of receive coil windings 1080.

In embodiments of the invention, ferrite disk(s) 1078 may comprise a single layer of ferrite material. In embodiments of the invention ferrite disk(s) 1078 may be a ferrite powder embedded in a rubbery matrix. In embodiments of the invention the ferrite layers, ferrite disks or ferrite rings described herein may be made of any suitable ferromagnetic material.

In embodiments of the invention, the present invention is directed to a contact hearing system including: a transmit coil positioned in an ear tip wherein the transmit coil includes an electrical coil wound on a ferrite core; a receive coil positioned on a contact hearing device wherein the receive coil includes an electrical coil wound on a non-ferrite core. In embodiments of the invention, the non-ferrite core includes air. In embodiments of the invention, the receive coil is a component of a receive coil assembly, the receive coil assembly including at least one ferrite disk positioned at a distal end of the receive coil. In embodiments of the invention, the at least one ferrite disk includes a hole in a center of the at least one ferrite spacer. In embodiments of the invention, the at least one ferrite disk includes a plurality of ferrite disks laminated together. In embodiments of the invention, the at least one ferrite disk includes two or more ferrite disks. In embodiments of the invention, the receive coil includes a first central axis and the at least one ferrite disk includes a second central axis, the first central axis and the second central axis being aligned. In embodiments of the invention, the ferrite core includes a channel extending from a proximal to a distal end thereof. In embodiments of the invention, a central axis of the transmit coil and a central axis of the receive coil are substantially parallel when the ear tip and the contact hearing device are positioned in an ear canal of a user. In embodiments of the invention, a central axis of the transmit coil and a central axis of the receive coil form an angle of approximately 15 degrees or less when the ear tip and the contact hearing device are positioned in an ear canal of a user. In embodiments of the invention, a central axis of the transmit coil and a central axis of the receive coil form an angle of approximately 25 degrees or less when the ear tip and the contact hearing device are positioned in an ear canal of a user. In embodiments of the invention, a distal end of the transmit coil is positioned within between three and seven millimeters of the proximal end of the receive coil.

In embodiments of the invention, the present invention is directed to a contact hearing system, the contact hearing system including: an ear tip, the ear tip including a transmit coil wherein the transmit coil is wound around a core including, at least in part, a ferromagnetic material; and a contact hearing device including a receive coil wherein the receive coil is wound around a core including, at least in part, a non-ferromagnetic material. In embodiments of the invention, the ferromagnetic material includes a ferrite material. In embodiments of the invention, the non-ferromagnetic material includes air. In embodiments of the invention, the contact hearing device includes a receive circuit assembly, the receive circuit assembly including: the receive coil; a disk attached to a distal end of the receive coil wherein the disk includes a ferromagnetic material. In embodiments of the invention, the disk has a diameter which is substantially the same as a diameter of the receive coil. In embodiments of the invention, the disk has a hole in its center. In embodiments of the invention, the receive circuit assembly further includes a printed circuit board including electronic components. In embodiments of the invention, the disk acts as a spacer to separate the printed circuit board from a distal end of the receive coil. In embodiments of the invention, magnetic flux lines entering a proximal end of the receive coil are bent away from the printed circuit board by the disk as they exit a distal end of the receive coil. In embodiments of the invention, at least a portion of magnetic flux lines entering a proximal end of the receive coil are prevented from reaching the printed circuit board as they exit a distal end of the receive coil. In embodiments of the invention, the presence of the disk increases a quality factor (Q) of the receive circuit assembly. In embodiments of the invention, the disk reduces eddy currents in conductive traces on the printed circuit board when magnetic flux is passed through the receive coil. In embodiments of the invention, the printed circuit board includes components of a demodulation circuit.

In embodiments of the invention, the transmit and/or receive coils may be encapsulated using a parylene coating.

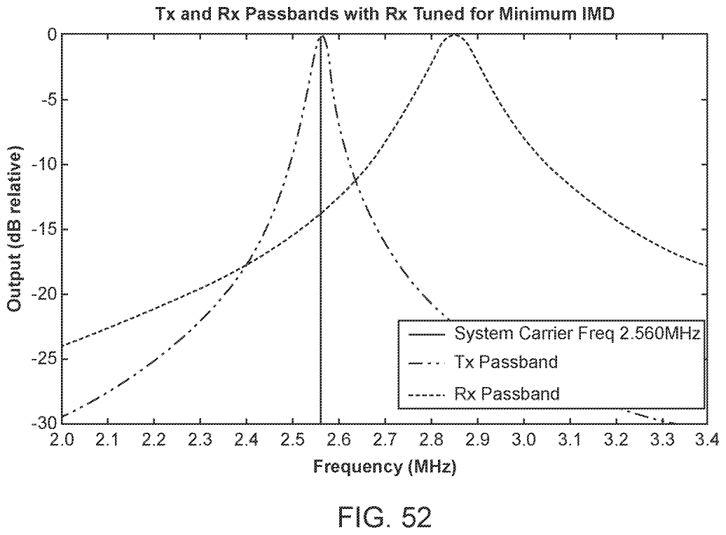

In embodiments of the invention, the Q (where Q is defined as the ratio of the energy stored in the resonator to the energy supplied by a to it, per cycle, to keep signal amplitude constant, at a frequency where the stored energy is constant with time) of the transmit circuit (“Tx Q”) is higher than the Q of the contact hearing device (“Rx Q”). In embodiments of the invention, the Tx Q may be greater than or equal to 70 and the Rx Q may be less than or equal to 20. In embodiments of the invention, the Rx Q is maximized by moving all circuitry to a board outside of the Rx coil. In embodiments of the invention, a ferrite core is used to increase the Q of the transmit coil. In embodiments of the invention, the transmit signal is amplified by exciting the transmit coil to a high state of resonance. FIG. 52 is an illustration of a system according to the present invention wherein a transmit circuit according to the present invention is tuned to have a higher Q than a receive circuit according to present invention. In embodiments of the invention, the transmit circuit may have a Q of between approximately 50 and 75. In embodiments of the invention, the transmit circuit may have a Q of approximately 60. In embodiments of the invention, the receive circuit may have a Q of between approximately 15 and 25.

In one embodiment, the present invention is directed to a contact hearing system including: an ear tip including a transmit circuit having a first Q value, wherein the ear tip includes a transmit coil wound on a ferrite core; a contact hearing device including a receive circuit having a second Q value, wherein the first Q value is greater than the second Q value; a receive coil positioned on the contact hearing device, wherein the receive coil includes a core of a non-ferromagnetic material. In embodiments of the present invention, the first Q value is greater than the second Q value by a factor of at least two. In embodiments of the present invention, the receive coil includes a disk including a ferromagnetic material at a distal end thereof. In embodiments of the present invention, the disk includes a ferrite material. In embodiments of the present invention, the disk includes a hole in its central portion. In embodiments of the present invention, the transmit coil is inductively coupled to the receive coil. In embodiments of the present invention, the contact hearing device includes a diode detector connected to the receive coil. In embodiments of the present invention, the contact hearing device includes a balanced armature microactuator connected to the receive coil. In embodiments of the present invention, the contact hearing device includes a platform which supports the receive coil, wherein the platform conforms to the anatomy of the wearers ear canal. In embodiments of the present invention, the contact hearing device includes a platform which supports the receive coil, wherein the platform is adapted to position the contact hearing device on a wearer's tympanic membrane.

In one embodiment, the present invention is directed to a method of inductively coupling an ear tip having a transmit circuit to a contact hearing device having a receive circuit, wherein the transmit circuit has a first Q value and the receive circuit has a second Q value, the first Q value being greater than the second Q value, the method including the steps of: exciting a transmit coil in the transmit circuit, the transmit coil being positioned in an ear tip; radiating an electromagnetic field from the transmit coil to a receive coil; receiving the radiated electromagnetic field at the receive coil, the receive coil being positioned on a contact hearing device; and transmitting information from the transmit coil to the receive coil using the electromagnetic field. In embodiments of the present invention, the first Q value is at least twice as large as the second Q value. In embodiments of the present invention, the transmit coil includes a ferrite core. In embodiments of the present invention, the receive coil includes a ferrite disk at a distal end thereof. In embodiments of the present invention, ferrite disk includes a hole in its central portion. In embodiments of the present invention, the information is transmitted from the transmit coil to the receive coil using near field radiation. In embodiments of the present invention, the transmit coil is inductively coupled to the receive coil. In embodiments of the present invention, the electromagnetic radiation induces a current in the receive coil. In embodiments of the present invention, the current induced in the receive coil is proportional to a level of magnetic flux passing through the receive coil. In embodiments of the present invention, a current induced in the receive coil drives a balanced armature microactuator positioned on the contact hearing device.

In one embodiment, the present invention is directed to a contact hearing system including: an ear tip including a transmit circuit having a first Q value, wherein the ear tip includes a transmit coil wound on a ferrite core, the first Q being in a range of between fifty and seventy-five; a contact hearing device including a receive circuit having a second Q value, wherein the second Q value is in the range of between fifteen and twenty-five; a receive coil positioned on the contact hearing device, wherein the receive coil has a core of non-ferromagnetic material. In embodiments of the present invention, the receive coil is a component of a receive circuit assembly, the receive circuit assembly including a disk at a distal end of the receive coil, wherein the disk includes a ferromagnetic material. In embodiments of the present invention, the receive coil assembly further includes a printed circuit board, the printed circuit board being separated from the distal end of the receive coil by the disk.

In a standard systems for transmitting information using electromagnetic waves it would be conventional to design the system such that both the transmit and receive circuits were optimized around the carrier frequency, that is that the transmitter would have its highest output at the carrier frequency and the receive circuit would have its most efficient reception at the carrier frequency (e.g. the receive coil or antenna would be optimized to pass signals at the carrier frequency with the least loss). In such a system it would be conventional to tune the transmitter (Tx) and receiver (Rx) resonance, to maximize power transfer. For example, you would tune both circuits to have a maximum Q with the pass band for both the Tx and Rx centered around the carrier frequency. Resonance generally occurs at (Where L is inductance and C is capacitance):

$${f}_{0} = \frac{{ω}_{0}}{2 π} = \frac{1}{2 π \sqrt{LC}} .$$

Where AM modulation is used, such as in inductively coupled systems according to the present invention, that standard tuning may result in Intermodulation Distortion and/or harmonic distortion. Intermodulation Distortion (IMD) may be defined as the ratio (in dB) between the power of fundamental tones and third-order distortion products which may, under certain circumstances be audible to a listener, for example, a hearing aid user. In a system such as a contact hearing, system IMD may manifest itself as distortion of words and letters which incorporate higher frequency tones (e.g. “S” and “T” sounds). This is a particular problem in such systems because contact hearing systems transmit and deliver those sounds directly to the tympanic membrane through mechanical manipulation of the tympanic membrane, unlike conventional hearing aids.

In embodiments of the present invention, it may be possible to reduce or eliminate such intermodulation distortion by tuning the receive coil to center the passband at a frequency above the frequency of the carrier. In embodiments of the invention, the center of the receive passband may be tuned to approximately 137 KHz above the carrier frequency. In embodiments of the invention, the center of the bandpass may be tuned to approximately 322 KHz above the carrier frequency. Thus, by tuning the Rx circuit in a manner which would be expected to result in lower efficiency (power transfer), the present invention reduces or eliminates intermodulation distortion. In embodiments of the invention, the Rx circuit is tuned such that the new center of the passband is above the carrier frequency while the transmit (Tx) circuit is tuned such that the center of the passband for the transmit (Tx) circuit is below the transmit frequency.

FIG. 52 is a graph showing passband tuning according to the present invention for a transmit circuit and a receive circuit according to the present invention. In FIG. 52 a transmit circuit is tuned such that the center of its passband is at the system carrier frequency (e.g. 2.560 MHz), while the receive passband is tuned such that the center of its passband is at a second, higher, frequency (e.g. 2.852). Further, as illustrated in FIG. 52 the transmit circuit is tuned to have a higher Q than the receive band. In embodiments of the invention, the transmit and received circuits are tuned to have an offset between the center of the transmit passband and the center of the receive passband in order to improve intermodulation distortion. In embodiments of the invention, the transmit and received circuits are tuned to have an offset between the center of the transmit passband and the center of the receive passband in order to improve power transmission from the transmitter to the receiver. In embodiments of the invention, the transmit and received circuits are tuned to have an offset between the center of the transmit passband and the center of the receive passband in order to increase output power at the contact hearing device. In embodiments of the invention, the center frequencies of the receive passband may be lower than the center frequency of the transmit passband.

In embodiments of the invention the relationship between the transmit passband and the receive passband may be such that a signal at a frequency which is at the center of the transmit passband (e.g. a carrier signal) would be attenuated by between approximately 10 dB and 15 dB if it were passed through a filter having the characteristics of the receive passband. In embodiments of the invention the relationship between the transmit passband and the receive passband may be such that a signal at a frequency which is at the center of the receive passband would be attenuated by between approximately 20 dB and 25 dB if it were passed through a filter having the characteristics of the receive passband.

In embodiments of the invention, the present invention is directed to a contact hearing system including: a transmit circuit including a transmit coil positioned in an ear tip, THE transmit circuit having a first bandpass characteristic, wherein the transmit circuit is tuned such that a center of the first bandpass characteristic is set at a first frequency; and a receive circuit including a receive coil positioned on a contact hearing device, the receive circuit having a second bandpass characteristic, wherein the receive circuit is tuned such that a center of the second bandpass characteristic differs from the center of the first bandpass characteristic. In embodiments of the invention, the transmit circuit is tuned such that the center of the first bandpass characteristic is a transmit carrier frequency. In embodiments of the invention, the transmit carrier frequency is approximately 2.56 MHz. In embodiments of the invention, the receive circuit is tuned such that the center of the second bandpass characteristic is tuned to a frequency which is higher than the first frequency. In embodiments of the invention, the receive circuit is tuned such that the center of the second bandpass characteristic is tuned to a frequency above a transmit carrier frequency. In embodiments of the invention, the receive circuit is tuned such that the center of the second bandpass characteristic is tuned to approximately 2.852 MHz. In embodiments of the invention, the receive circuit is tuned such that the center of the second bandpass characteristic is tuned to a frequency within 5 percent of the carrier frequency. In embodiments of the invention, the receive circuit is tuned such that the center of the second bandpass characteristic is tuned to a frequency within 10 percent of the carrier frequency. In embodiments of the invention, the receive circuit is tuned such that the center of the second bandpass characteristic is tuned to a frequency which is within the bandpass characteristics of the transmit circuit.

In embodiments of the invention, the present invention is directed to a contact hearing system including: a transmit circuit including a transmit coil positioned in an ear tip, the transmit circuit having a first passband, wherein the transmit circuit is tuned such that a center of the first passband is set at a first frequency; and a receive circuit including a receive coil positioned on a contact hearing device, the receive circuit having a second passband, wherein the receive circuit is tuned such that a center of the second passband differs from the center of the first passband.

In embodiments of the invention, the present invention is directed to a contact hearing system including: a transmit circuit including a transmit coil positioned in an ear tip, the transmit circuit having a first bandpass characteristic, wherein the transmit circuit is tuned such that a center of the first bandpass characteristic is set at a first frequency; a receive circuit including a receive coil positioned on a contact hearing device, the receive circuit having a second bandpass characteristic, wherein the receive circuit is tuned such that a center of the second bandpass characteristic differs from the center of the first bandpass characteristic; and wherein the receive circuit is tuned such that the center of the second bandpass characteristic is tuned to a frequency which is lower than the first frequency. In embodiments of the invention the receive circuit is tuned such that the center of the second bandpass characteristic is tuned to a frequency below a transmit carrier frequency. In embodiments of the invention the receive circuit is tuned such that the center of the second bandpass characteristic is tuned to a frequency within 5 percent of the carrier frequency. In embodiments of the invention, the receive circuit is tuned such that the center of the second bandpass characteristic is tuned to a frequency within 10 percent of the carrier frequency. In embodiments of the invention, the receive circuit is tuned such that the center of the second bandpass characteristic is tuned to a frequency which is within the bandpass characteristics of the transmit circuit.

In embodiments of the invention, the present invention is directed to a method of tuning a transmit circuit and a receive circuit, wherein the transmit and receive circuit form components of a contact hearing system, the transmit circuit having a bandpass characteristic and the receive circuit having a bandpass characteristic, the method including the steps of tuning the bandpass characteristics of the transmit circuit such that a center of the transmit bandpass characteristic is set to a first frequency; and tuning the bandpass characteristics of the receive circuit such that a center of the receive bandpass characteristic is set to a second frequency, the second frequency differing from the first frequency. In embodiments of the invention, second frequency it higher than the first frequency. In embodiments of the invention, the first frequency is the transmit carrier frequency. In embodiments of the invention, the first frequency is approximately 2.56 MHz. In embodiments of the invention, the transmit circuit includes a transmit coil wound on a ferrite core, the transmit coil and ferrite core being positioned in an ear tip. In embodiments of the invention, the receive circuit includes a receive coil positioned on a contact hearing device. In embodiments of the invention, the transmit circuit and the receive circuit are adapted to be positioned in the ear canal of a user. In embodiments of the invention, the first frequency is selected to be less than 10% lower than the second frequency. In embodiments of the invention, the first frequency is selected to be less than 5 percent lower than the first frequency. In embodiments of the invention, the second frequency is within the bandpass characteristics of the transmit circuit. In embodiments of the invention, the second frequency is selected such that, if passed through a filter having the bandpass characteristics of the transmit circuit it would be attenuated by less than six decibels. In embodiments of the invention, the second frequency is selected such that, if passed through a filter having the bandpass characteristics of the receive circuit it would be attenuated by less than three decibels.

In embodiments of the invention the present invention is directed to a method of tuning a transmit circuit and a receive circuit, wherein the transmit and receive circuit form components of a contact hearing system, the transmit circuit having a passband and the receive circuit having a passband, the method including the steps of: tuning the passband of the transmit circuit such that a center of passband of the transmit circuit is set to a first frequency; and tuning the bandpass characteristics of the receive circuit such that a center of the passband of the receive circuit is set to a second frequency, the second frequency differing from the first frequency.

In embodiments of the invention, the present invention is directed to a method of tuning a transmit circuit and a receive circuit, wherein the transmit and receive circuit form components of a contact hearing system, the transmit circuit having a bandpass characteristic and the receive circuit having a bandpass characteristic, the method including the steps of tuning the bandpass characteristics of the transmit circuit such that the center of the bandpass is set to a first frequency; and tuning the bandpass characteristics of the receive circuit such that the center of the bandpass is set to a second frequency, the second frequency differing from the first frequency wherein the second frequency is lower than the first frequency. In embodiments of the invention, the first frequency is a transmit carrier frequency. In embodiments of the invention, the transmit circuit includes a transmit coil wound on a ferrite core, the transmit coil and ferrite core being positioned in an ear tip. In embodiments of the invention, the receive circuit includes a receive coil positioned on a contact hearing device. In embodiments of the invention, the transmit circuit and the receive circuit are adapted to be positioned in an ear canal of a user. In embodiments of the invention, the first frequency is selected to be less than 10% lower than the second frequency. In embodiments of the invention, the first frequency is selected to be less than 5 percent lower than the first frequency. In embodiments of the invention, the second frequency is within the bandpass characteristics of the transmit circuit. In embodiments of the invention, the second frequency is selected such that, if passed through a filter having the bandpass characteristics of the transmit circuit it would be attenuated by less than six decibels. In embodiments of the invention, the second frequency is selected such that, if passed through a filter having the bandpass characteristics of the transmit circuit it would be attenuated by less than three decibels. In embodiments of the invention, the second frequency is selected such that, if passed through a filter having the bandpass characteristics of the transmit circuit it would be attenuated by between 20 and 25 decibels. In embodiments of the invention, the first frequency is within the bandpass characteristics of the receive circuit. In embodiments of the invention, the first frequency is selected such that, if passed through a filter having the bandpass characteristics of the receive circuit it would be attenuated by between 10 and 15 decibels. In embodiments of the invention: the second frequency is selected such that, if passed through a filter having the bandpass characteristics of the transmit circuit it would be attenuated by between 20 and 25 decibels; and the first frequency is selected such that, if passed through a filter having the bandpass characteristics of the receive circuit it would be attenuated by between 10 and 15 decibels.

In embodiments of the invention, signals may be transmitted between the ear tip and the contact hearing device using an amplitude modulated oscillating magnetic field with a 2.5 MHz carrier frequency. In embodiments of the invention, the digital audio signal generated by the audio processor may be mixed with a carrier at the desired coupling frequency. In embodiments of the invention, the coupling circuit including the transmit coil subsystem and the receive coil subsystem may act as a band pass filter and the resulting waveform is an AM modulated signal which may be detected by the diode circuit connected to the receive coil. In embodiments of the invention, driver circuit may be a type D (H Bridge) and the mixing may be accomplished using an AND or a NAND gate with the carrier and the delta sigma digital modulation signal (the output of the delta sigma modulator, which may be a digital stream representative of an audio signal). In embodiments of the invention, the two legs of the H Bridge may be driven 180 degrees out of phase. In embodiments of the invention, the second leg may be driven by just the inverted (with respect to the other leg) carrier signal, allowing independent control of an additional carrier signal. This additional carrier may be used to overcome distortion caused by the non-linear current-voltage relationship of the diodes near the forward voltage Vf without sacrificing the dynamic range of the delta sigma modulator. The carrier leg voltage source can be independently controlled to adjust the amount of additional carrier inserted. In embodiments of the invention, the modulation may be FM or Frequency Modulation.

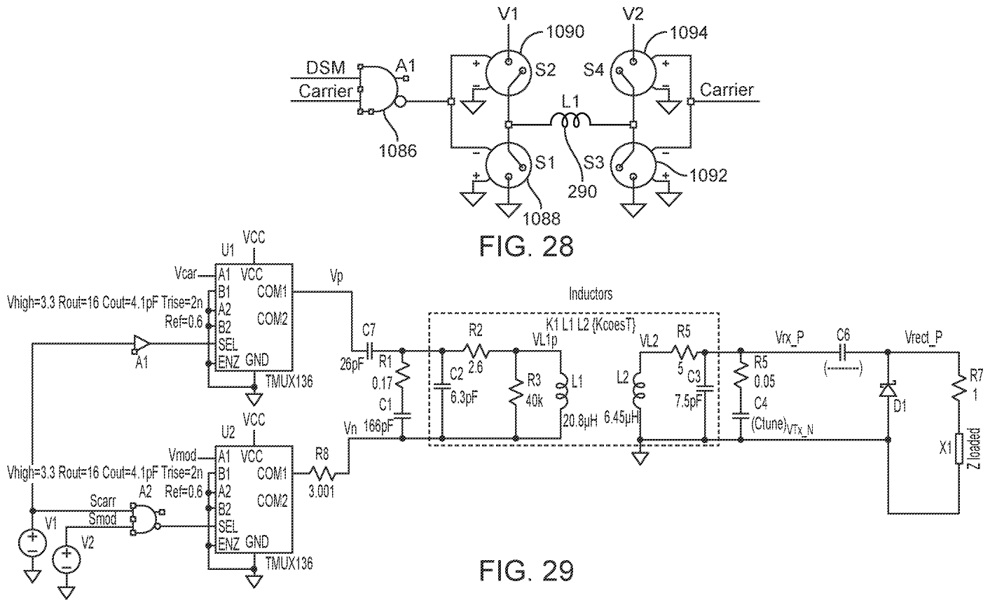

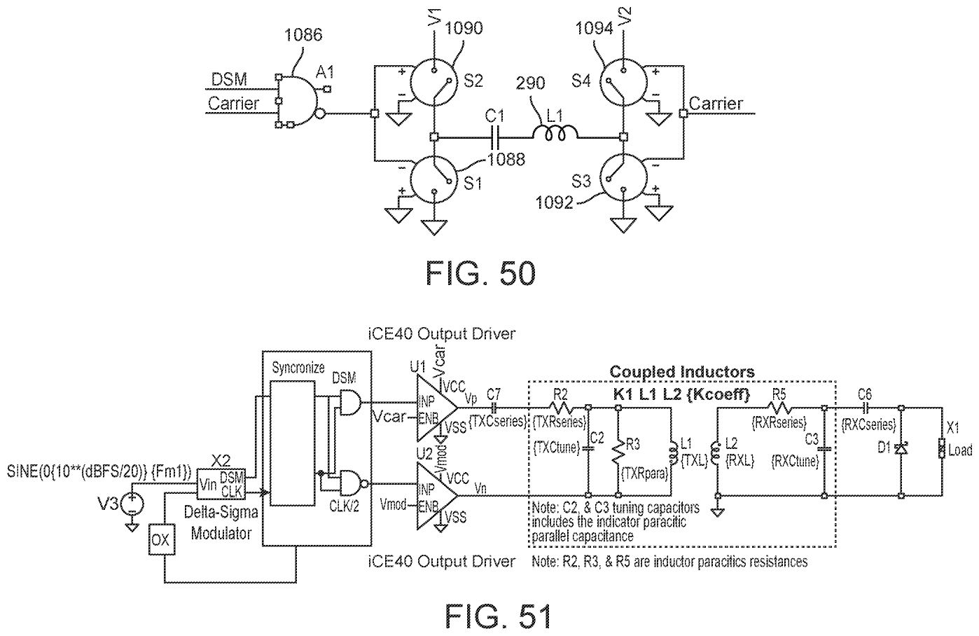

FIGS. 28, 49, and 50 are illustrations of a circuit that uses a delta sigma modulator (DSM) signal input to modulate a carrier signal in a standard H-Bridge configuration. In embodiments of the invention each side is driven 180 degrees out of phase with respect to each other. In FIGS. 28, 49, and 50, the H-Bridge circuit may comprise one or more AND/NAND circuits 1086, in combination with switches 1088, 1090, 1092 and 1094. The output of the H-Bridge may be supplied to transmit coil 290 to provide an AM signal field which may transmitted to a receive coil 130 by, for example, inductively coupling the output of transmit coil 290 to receive coil 130. In embodiments of the invention, illustrated in FIGS. 49 and 50, one side of the H-Bridge may be coupled to transmit coil 290 through a capacitor C1 Modulation is accomplished using the multiplicative property of the AND/NAND function. FIGS. 28, 49, and 50 are illustrations of a circuit according to the present invention wherein the DSM input is the delta sigma digital modulation signal. In embodiments of the invention, the output of the delta-sigma modulator is a signal representative of the sound received by the processor which is to be transmitted to the contact hearing device by amplitude modulation (AM) of the carrier. In FIGS. 28, 49, and 50, the carrier may be a clock signal. In FIGS. 28, 49, and 50, the carrier clock signal may be twice the DSM rate. FIGS. 28, 49, and 50, the carrier may be a digital clock signal representative of the carrier frequency, for example, 2.5 MHz. In embodiments of the invention, switches S1-S4 may be solid state/digital switches, such as, FET transistors. In embodiments of the invention, S1-S4 may form an H Bridge input to a resonant circuit (capacitor C1 and inductor L1). In embodiments of the invention, the AM output signal may be formed by filtering the differential digital signal using a resonator. L1 may be the transmit coil 290. In the embodiment of the invention illustrated in FIGS. 28 and 50, the input to a first side of the H Bridge is the NAND output of the AND/NAND gate circuit which has as its inputs the DSM signal and the Carrier signal. In the embodiment of the invention illustrated in FIG. 49, the input to a first side of the H Bridge is the NAND output of the AND/NAND gate circuit which has as its inputs the DSM signal and the Carrier signal while the input to a second side of the H Bridge is the AND output of the AND/NAND gate circuit which has as its inputs the DSM signal and the Carrier signal. In embodiments of the invention, V1 supply voltage controls the amount of modulated signal power transmitted. In embodiments of the invention, V2 supply controls the additional carrier power. In embodiments of the invention, the AND/NAND gate(s) serves as the mixer, multiplying the modulation signal and the carrier, creating an AM modulation of the carrier.

FIGS. 29 and 51 are system models of a system according to the present invention, including transmission and receive tank circuits and a detector circuit. In embodiments of the invention, key components (e.g. the AND and NAND function along with the synchronization of the Pulse Density Modulation (PDM) with the clock) may be implemented using a Field Programmable Gate Array (FPGA). Further, the low capacitance FPGA output driver (for example, an iCE40 Output Driver from Lattice Semiconductor) may be used to create the H-Bridge. In embodiments of the invention time skews and clock jitter can be kept at a minimum by re-clocking the outputs after the logic and just prior to the output driver. In embodiments of the invention, the circuit illustrated in FIGS. 29 and 51 may include discrete components to model the parasitic elements in the primary component. For example, R2, R3 and C2 may be the parasitic resistance and capacitance in L1.

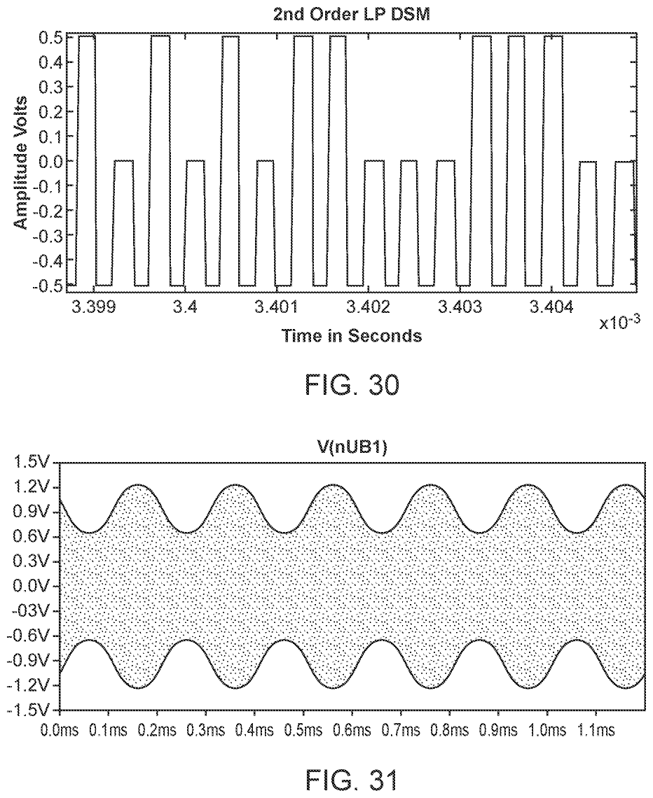

FIG. 30 illustrates the time domain waveform when the added carrier clock is the same size as the delta sigma signal mixed with the carrier clock.

FIG. 31 illustrates the resulting waveform with a 95% delta sigma with the added clock. Note that this is an AM signal with a modulation index of less than 50%. The embodiment illustrated in FIG. 31 may result in a lower distortion when using a simple diode detector while leaving the full dynamic range of the delta sigma modulator.

Several alternative methods of generating additional carrier exist. In embodiments of the invention, the signal could be generated using conventional analog means (mixer) then sum in additional carrier. In embodiments of the invention, the signal may be generated by digitally generating the desired waveform (including the added carrier) then using a high speed DAC (Digital to Analog converter. In embodiments of the invention, the mixing could also be performed by modulating the supply voltage to the H Bridge. In embodiments of the invention, this method could also be used to make a very simple cost effective AM modulator and by reversing the phase of the added carrier, suppressing the carrier double sideband suppressed carrier DSBSC. For standard AM the second leg of the H Bridge would be inverted from the first.

In one embodiment, the present invention is directed to a contact hearing system including: an ear tip including a transmit coil, wherein the transmit coil is connected to an audio processor, including an H Bridge circuit; a first input to the H Bridge circuit including an AND circuit wherein a first input to the AND circuit includes a carrier signal and a second input to the AND circuit includes an output of a delta sigma modulation circuit, wherein the delta sigma modulation circuit is a component of the audio processor; and a second input to the H Bridge circuit including an NAND circuit wherein a first input to the NAND circuit includes a carrier signal and a second input to the NAND circuit includes an output of the delta sigma modulation circuit. In embodiments of the invention, an output of a first side of the H Bridge circuit is connected to a first side of the transmit coil and an output of a second side of the H Bridge circuit is connected to a second side of the transmit coil. In embodiments of the invention, a capacitor is connected between at least one output of the H Bridge circuit and the transmit coil. In embodiments of the invention, the transmit coil is inductively coupled to a receive coil. In embodiments of the invention, the receive coil is positioned on a contact hearing device. In embodiments of the invention, the contact hearing device includes a diode detector connected to an output of the receive coil.

In one embodiment, the present invention is directed to a method of transmitting signals between a transmitter and receiver in an inductively coupled contact hearing system, the method including the steps of: mixing an output of a delta sigma modulation circuit with a carrier signal using an AND gate; providing an output of the AND gate to a first input of an H Bridge circuit; mixing the output of the delta sigma modulation circuit with the carrier signal using an NAND gate; providing an output of the NAND gate to a second input of the H Bridge circuit; providing an output of a first side of the H Bridge circuit to a first side of a transmit coil; and providing an output of a second side of the H Bridge circuit to a second side of the transmit coil. In embodiments of a method according to the present invention the method further including the steps of: receiving a signal generated by the transmit coil at a receive coil; passing the received signal through a diode detector. In embodiments of a method according to the present invention, the method further includes the step of: passing the output of the diode detector to a balanced armature transducer. In embodiments of the invention the carrier is AM modulated. In embodiments of the invention, the diode detector demodulates the AM modulated carrier.

In one embodiment, the present invention is directed to a contact hearing system including: an ear tip including a transmit coil, wherein the transmit coil is connected to an audio processor, including an H Bridge circuit, wherein the transmit coil is connected to the output of the H Bridge circuit; a first input to the H Bridge circuit including an AND circuit wherein a first input to the AND circuit includes a carrier signal and a second input to the AND circuit includes an output of a delta sigma modulation circuit, wherein the delta sigma modulation circuit is a component of the audio processor; and a second input to the H Bridge circuit including the carrier signal. In embodiments of the invention, the second input is an inverted carrier signal. In embodiments of the invention, the transmit coil is inductively coupled to a receive coil. In embodiments of the invention, the receive coil is positioned on a contact hearing device. In embodiments of the invention, the contact hearing device includes a diode detector connected to an output of the receive coil.

In one embodiment, the present invention is directed to a method of transmitting signals between a transmitter and receiver in an inductively coupled contact hearing system, the method including the steps of: mixing the output of a delta sigma modulation circuit with a carrier signal using an AND gate; providing an output of the AND gate to a first input of an H Bridge circuit; providing a carrier signal to a second input of an H Bridge circuit; providing an output of a first side of the H Bridge circuit to a first side of a transmit coil; and providing an output of a second side of the H Bridge circuit to a second side of a transmit coil. In a method according to the present invention, the method further includes the steps of: receiving a signal generated by the transmit coil at a receive coil; passing the received signal through a diode detector. In a method according to the present invention, the method further includes the steps of: passing an output of the diode detector to a balanced armature transducer. In embodiments of the invention, the carrier is AM modulated. In embodiments of the invention, the diode detector demodulates the AM modulated carrier signal.

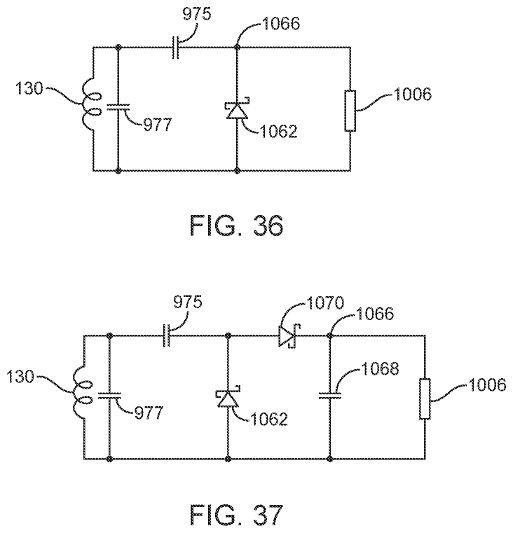

As described earlier, a Villard, 1-diode demodulator, such as, for example the circuit illustrated in FIG. 36 may be used as a demodulator circuit in embodiments of the present invention. In the circuit illustrated in FIG. 36, receive coil 130 has an inductance L-Rx, which forms a tank resonator when used in combination with resonance capacitor 977, having a tuning capacitance C-tune, which may be modified by the combined capacitances of the remaining circuit components, including AC filter capacitor 975 (which may be a series capacitor), Schottky diode 1062 and load 1006 (which may be, for example, a microactuator). In embodiments of the invention, resonance capacitor 977 may have a capacitance C-tune which is composed of 3 0201-size capacitors that are chosen to make the tank circuit resonate near or at the carrier frequency of approximately 2.5 MHz. In embodiments of the invention the carrier frequency may be approximately 2.560 MHz. C-tune, in combination with the other components and, in particular, the Villard 1-diode demodulator may be chosen to provide a high output while minimizing intermodulation distortion (IMD).

In the embodiment of the invention, illustrated in FIG. 36, a signal received by contact hearing device 112 and, on a negative half cycle of the carrier voltage, charge enters motor node 1066 through first diode 1062, which may be, for example, a Schottky diode. On the subsequent positive half-cycle of the carrier, AC filter capacitor 975 and first diode 1062 holds this charge on motor node 1066 while displacement current travels through AC filter capacitor 975 into motor node 1066. This sequence results in a voltage doubling at motor node 1066 on each carrier cycle. While acting as an efficient demodulator, a Villard circuit of the kind described may result in large voltage peaks at motor node 1066, which large peaks may result in distortion, such as intermodulation distortion.

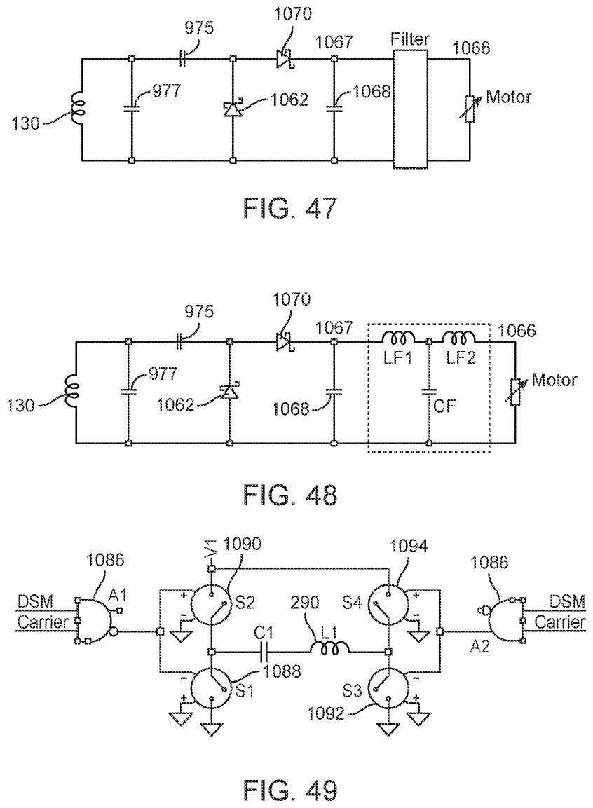

In the embodiment of the invention, illustrated in FIGS. 37, 47 and 48 a Greinacher circuit may be used to demodulate a signal received by contact hearing device 112. In embodiments of the invention, the Greinacher circuit may be a Villard circuit followed by a peak detector, wherein the peak detector may comprise a second diode 1070 (which may in some embodiments of the invention, be a Schottky diode) and a smoothing capacitor 1068. The extra diode and capacitor act to smooth out the sharp voltage peaks on the Villard output. In a contact hearing device according to the present invention, smoothing capacitor 1068 may be used to present a more consistent output to load 1006. In embodiments of the invention, smoothing capacitor 1068 may form a tank circuit with load 1006 wherein the presence of the tank circuit boosts the output of contact hearing device at frequencies around 10 kHz. In embodiments of the invention, smoothing capacitor 1068 may form a tank circuit with load 1006 wherein the presence of the tank circuit boosts the output of contact hearing device at the high end of the range of frequencies of interest (e.g. around 10 kHz). In embodiments of the invention, smoothing capacitor 1068 may form a tank circuit with load 1006 wherein the presence of the tank circuit boosts the output of contact hearing device at frequencies around 10 kHz, thereby ensuring that the output of contact hearing device 112 is substantially level across the range of frequencies of interest (e.g. from 100 Hz to 10,000 Hz) and does not fall off as the frequency approaches the higher end of the band. In embodiments of the invention, the circuit illustrated in FIG. 37 may be used to both minimize intermodulation distortion and maintain the output of contact hearing device 112 up to a frequency of approximately 10,000 Hz. In the embodiment of FIG. 47, the Greinacher (2-diode) circuit includes an output filter. In the embodiment of FIG. 48, the Greinacher (2-diode) circuit includes an LC output filter.

FIGS. 47 and 48 are illustrations of circuits used to implement a Greinacher demodulator with filter according to the present invention. In embodiments of the invention, the filter is intended to prevent (reduce) the carrier RF reaching the load (motor). In embodiments of the invention, the filter implementation is preferably low-pass, allowing the audio signals to pass with minimal attenuation up to 10 kHz while reducing/blocking the RF at 2.56 MHz or any carrier frequency. In embodiments of the invention, intermodulation distortion (IMD) is improved (around 5 dB) with two types of low-pass filters in this position. In embodiments of the invention, IMD may be improved by the addition of a filter to the Greinacher demodulator. In embodiments of the invention, the presence of the filter may reduce the amount of RF voltage on diode output node 1067 which reaching motor node 1066, which will reduce the reflected RF from the motor from reaching the diodes (especially D2 at diode output node 1067) and mixing with the original signal. Mixing of signals in the diode, because of the non-linear I-V curve, results in distortion and may be the main contribution to IMD.

In embodiments of the invention, Villard (single diode) demodulation circuits may be used to increase the efficiency of the contact hearing device as they use a single diode which is only turned on for one half cycle. Unfortunately, Villard demodulation circuits produce larger spikes as they also act as voltage doublers. In demodulation circuits of this kind, the number of diodes in the circuit dictates its efficiency (in part) as the power needed to turn on a diode is not usable in signal transfer and is, therefore, lost. Greinacher (two diode) demodulation circuits have advantages over Villard demodulation circuits because the second diode of the Greinacher circuit in combination with smoothing capacitor 1068 smooths out the voltage and current spikes of the Villard, thus ensuring a smother demodulated signal and potentially reducing distortion. In addition, the Greinacher circuit is beneficial because it smooths out the response of the system across the frequency band of interest (in this case between approximately 100 Hz and 10,000 Hz such that the output of the demodulator is substantially the same across that range.

In embodiments of the invention, the present invention is directed to a contact hearing system including: a transmit coil positioned in an ear tip wherein the transmit coil includes an electrical coil wound on a ferrite core; a receive coil positioned on a contact hearing device wherein the receive coil includes an electrical coil without a core; a load connected to the receive coil; and a demodulation circuit connected to the receive coil and the load wherein the demodulation circuit includes a voltage doubler and a peak detector. In embodiments of the invention, the demodulation circuit is connected to the load at a motor node. In embodiments of the invention, a tuning capacitor is connected across the receive coil. In embodiments of the invention, the voltage doubler includes a series capacitor connected to a first diode. In embodiments of the invention, the series capacitor is connected between a first side of the receive coil and a cathode of the first diode. In embodiments of the invention, the cathode of the first diode is connected to a second side of the receive coil. In embodiments of the invention, the peak detector is connected between an output of the voltage doubler and the load. In embodiments of the invention, the peak detector includes a second diode and a smoothing capacitor. In embodiments of the invention, an anode of the second diode is connected to the voltage doubler. In embodiments of the invention, a cathode of the first diode is connected to an anode of the second diode. In embodiments of the invention, a cathode of the second diode is connected to a first side of the smoothing capacitor. In embodiments of the invention, the cathode of the second diode and the first side of the smoothing capacitor is connected to a first side of the load. In embodiments of the invention, a second side of the load is connected to a second side of the smoothing capacitor. In embodiments of the invention, the first diode is a Schottky diode. In embodiments of the invention, the second diode is a Schottky diode. In embodiments of the invention, the load is a microactuator. In embodiments of the invention, the load is a balanced armature microactuator.

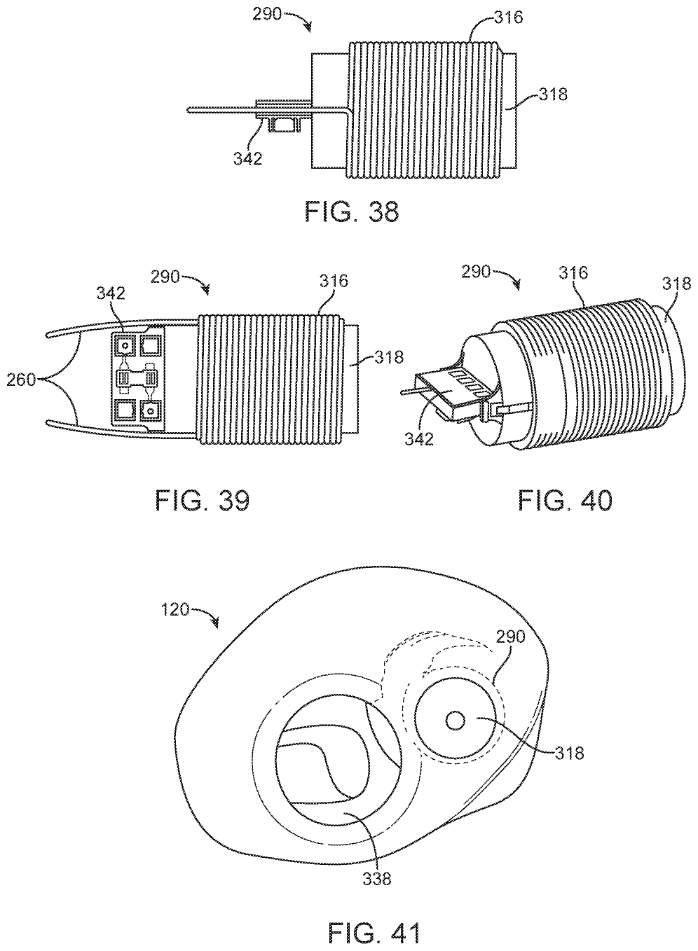

FIG. 38 is a side view of a transmit coil for use in an ear tip according to the present invention. FIG. 39 is a top view of a transmit coil for use in an ear tip according to the present invention. FIG. 40 is a side perspective view of a transmit coil for use in an ear tip according to the present invention. In the embodiments of the invention, illustrated in FIGS. 38-40, transmit coil 290 includes coil winding 316 which is wrapped around ferrite core 318, which, in the embodiments of FIGS. 38-40 may be a solid core with no acoustic vent. Transmit coil 290 may further include transmit electronics 342.

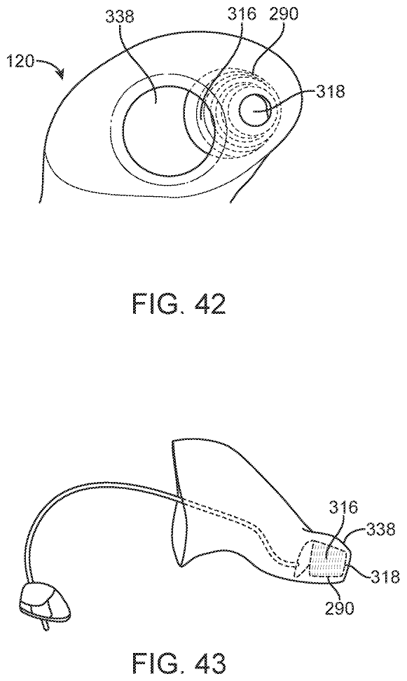

FIG. 41 is an end view of an ear tip according to the present invention. FIG. 42 is an end view of an ear tip according to the present invention. FIG. 43 is a side view of an ear tip assembly according to the present invention. In the embodiments of the invention, illustrated in FIGS. 41-43, ear tip 120 includes transmit coil 290 and acoustic vent 338. Transmit coil 290 may include coil winding 316 and ferrite core 316. In embodiments of the invention, ferrite core 316 may be constructed of a ferrite material or of any magnetic material. In embodiments of the invention, a distal end of ferrite core 316 may extend beyond a distal end of coil winding 316.

In embodiments of the invention described and claimed herein the text may refer to a “medial” or a “lateral” end or side of a device or component. In embodiments of the invention described and claimed herein, the text may refer to a “distal” or a “proximal” end or side of a device or component. In embodiments of the invention, “medial” and “distal” may refer to the side or end of the device or component which is farthest from the outside of the user's body (e.g. at the end of the ear canal where the tympanic membrane is found. In embodiments of the invention, “lateral” and “proximal” may refer to the side or end of the device or component which is closest to the outside of the user's body (e.g. at the open end of the ear canal where the pinna is found).

In embodiments of the present invention, biometric sensors and other devices may be placed in proximity to, on or in the ear canal resulting in a system with the ability to collect information on the user's environment, including information on the user's location, the time of day, and the activity the user is engaged in. In embodiments of the present invention, drug delivery devices may be placed in proximity to, on or in the ear canal resulting in a system with the ability to deliver drugs to a user through the ear and/or components of the ear. In embodiments of the present invention, the combination of a superior hearing system with biometric sensors and other devices, such as drug delivery devices, in a single system which may be placed in proximity to, on or in the ear canal may result in a system with the ability to collect information on the user's environment, including information on the user's location, the time of day, and the activity the user is engaged in.

The system may further provide access to highly vascular sections of ear canal, including the pars tensa and manubrium vessels and the information that may be gathered from such locations. The system may further provide the ability to gather data, monitor health, send alerts and deliver drugs through a device that is in place 24 hours a day for years on end, without interfering with or changing the wearer's day to day activities. The system may further provide the ability to ensure user compliance without the need for user interaction, other than, in some cases, normal upkeep. In some embodiments, the current invention may be used to replace halter monitors, event recorders and/or Sub-Cutaneous (Sub-Q) monitors (e.g. injectable monitors). The system may further provide the ability to mount sensors directly against the skin and ensure that they stay in place over long periods of time, by, for example, using system components that are custom fit to the ear canal wall and/or to the tympanic membrane. The system may further provide the user with feedback, instructions or warnings which go directly to the wearer's tympanic membrane in a manner which is imperceptible to any third party.

A system according to the present invention may further enable a user to take advantage of characteristics of the ear canal of the user to make measurements of the user's biometric data, including: positioning of sensors in a place, which is undetectable to both the user and third parties; positioning of sensors in a place where they are well protected from the environment, and from external forces (not subject to false alarms, such as, for example, the type of false alarms that result from the dropping or shaking of externally worn devices); positioning of sensors in a very vascular environment; positioning sensors in an environment which may be highly conducive to the measurement of biometric data (an environment where a better signal to noise ratio is achievable—enclosed and dark to facilitate optical measurements; and positioning sensors in an environment where an extensive range of biometric data is available and can be measured, including blood pressure, heart rate, glucose levels, respiration rate, temperature, blood flow and other biometric data.

A system according to the present invention may further provide: the ability to deliver drugs to a user, including sustained, timed and/or algorithm controlled drug delivery; the ability to ensure compliance with drug regimens by automating drug delivery in an easily accessible region such as the ear canal; the ability to limit the amount of drug delivered without compromising efficacy by delivering to highly vascular tissue in or around the ear canal, such as, for example, the pars tensa and manubrium vessels; the ability to deliver drugs to regions of the body where the vasculature is easily accessible, for example, where the tissue covering the vasculature is very thin, such as, for example, over the manubrium vessels; the ability to locally deliver drugs which are normally delivered systemically, thereby reducing the amount of drugs delivered and the related side effects; and the ability to deliver drugs and treat diseases using a novel platform in the ear canal. Drugs which may be delivered using the present invention include antibiotics (neomycin/quinolenes), dexamethasone, steroids (prednisolone), acetic acid, aluminum acetate, boric acid, betnesol, prednisolone sodium phosphate, clotrimazole, Ceruminolytic agents (sodium chloride/chlorbutanol/paradichlorobenzene), amoxicillin, flucloxacillin; ciprofloxacillin, penicillin, betahistine dopamine antagonists (prochlorperazine), antihistamines (cinnarizine and cyclizine), antiviral drugs (acyclovir), sodium fluoride, nicotine and insulin. Diseases which may be treated using the present invention include acute otitis media, furunculosis of external auditory canal, perichondritis of pinna, acute mastoiditis, and malignant otitis externa, vertigo, herpes zoster oticus and cancer. Embodiments of the invention may be used to deliver drugs in which systemic or local drug delivery would be beneficial.

FIG. 1 shows a hearing system 10 conFIG.d to transmit electromagnetic energy EM to a medial ear canal assembly 100 positioned in the ear canal EC of the user. The ear comprises an external ear, a middle ear ME and an inner ear. The external ear comprises a Pinna P and an ear canal EC and is bounded medially by a tympanic membrane (also referred to as an eardrum) TM. Ear canal EC extends medially from pinna P to tympanic membrane TM. Ear canal EC is at least partially defined by a skin SK disposed along the surface of the ear canal. The tympanic membrane TM comprises a tympanic membrane annulus TMA that extends circumferentially around a majority of the eardrum to hold the eardrum in place. The middle ear ME is disposed between tympanic membrane TM of the ear and a cochlea CO of the ear. The middle ear ME comprises the ossicles OS to couple the tympanic membrane TM to cochlea CO. The ossicles OS comprise an incus IN, a malleus ML and a stapes ST. The malleus ML is connected to the tympanic membrane TM and the stapes ST is connected to an oval window OW, with the incus IN disposed between the malleus ML and stapes ST. Stapes ST is coupled to the oval window OW so as to conduct sound from the middle ear to the cochlea.

The hearing system 10 may include an input transducer assembly 20 and a medial ear canal assembly 100 to transmit sound to the user. Hearing system 10 may comprise a sound processor 24, which may be, for example, a behind the ear unit (BTE). Sound processor 24 may comprise many components of hearing system 10 such as a speech processor, battery, wireless transmission circuitry, and input transducer assembly 20. The input transducer assembly 20 can be located at least partially behind the pinna P or substantially or entirely within the ear canal EC. Input transducer assembly 20 may further comprise a Bluetooth™ connection to couple to a cell phone or other external communication device 26. The medial ear canal assembly 100 of hearing system 10 may comprise components to receive the light energy or other energy, such as RF energy and vibrate the eardrum in response to such energy.

The input transducer assembly 20 can receive a sound input, for example an audio sound or an input from external communication device 26. With hearing aids for hearing impaired individuals, the input can be ambient sound. The input transducer assembly may comprise at least one input transducer, for example a microphone 22. The at least one input transducer may comprise a second microphone located away from the first microphone, in the ear canal or the ear canal opening, for example positioned on sound processor 24. Input transducer assembly 20 may also include can include a suitable amplifier or other electronic interface. In some embodiments, the input may comprise an electronic sound signal from a sound producing or receiving device, such as a telephone, a cellular telephone, a Bluetooth connection, a radio, a digital audio unit, and the like.

Input transducer assembly 20 may include a lateral ear canal assembly 12 which may comprise a light source such as an LED or a laser diode for transmitting data (including audio data) and energy to medial ear canal assembly 100. In other embodiments, lateral ear canal assembly 12 may comprise an electromagnetic coil, an RF source, or the like for transmitting data (including audio data) and energy to medial ear canal assembly 100. In embodiments of the invention, lateral ear canal assembly 12 may further comprise a receiver adapted to receive data transmitted from medial ear canal assembly 100, such as, for example, biometric data from sensors positioned on or near medial ear canal assembly 100.

In embodiments of the invention, medial ear canal assembly 100 is adapted to receive the output from input transducer assembly 20 and produce mechanical vibrations in response to the received information, which may be, for example, in the form of a light signal generated by lateral ear canal assembly 12. In embodiments of the invention, medial ear canal assembly 100 comprises a sound transducer, wherein the sound transducer may comprise at least one of a microactuator, a coil, a magnet, a magnetostrictive element, a photostrictive element, or a piezoelectric element. In embodiments of the invention, input transducer assembly 20 may comprise a light source coupled to sound processor 24 by a fiber optic cable and positioned on lateral ear canal assembly 12. In embodiments of the invention, input transducer assembly 20 may comprise a laser diode coupled to sound processor 24 and positioned on lateral ear canal assembly 12. In embodiments of the invention, the light source of the input transducer assembly 20 may be positioned in the ear canal along with sound processor 24 and microphone 22. When properly coupled to the subject's hearing transduction pathway, the mechanical vibrations caused by medial ear canal assembly 100 can stimulate the cochlea CO, which induces neural impulses in the subject which can be interpreted by the subject as a sound input.

FIG. 2 and FIG. 3 show isometric and top views, respectively, of an embodiment of medial ear canal assembly 100 according to the present invention. In the illustrated embodiments, medial ear canal assembly 100 may comprise a retention structure 110, a support structure 120, a transducer 130, at least one spring 140, and a photodetector 150. Medial ear canal assembly 100 may include data processor 200 and transmitter 210 which may be positioned on transducer 130. Retention structure 110, which may be a resilient retention structure, may be sized to couple to the tympanic membrane annulus TMA and at least a portion of the anterior sulcus AS of the ear canal EC. Retention structure 110 may comprise an aperture 110A. Aperture 110A is sized to receive transducer 130 and to allow for normal transduction of sound through the subjects hearing transduction pathway.

The retention structure 110 can be sized to the user and may comprise one or more of an O-ring, a C-ring, a molded structure, or a structure having a shape profile so as to correspond to the user's ear canal anatomy, or to a mold of the ear canal of the user. Retention structure 110 may comprise a resilient retention structure such that the retention structure can be compressed radially inward as indicated by arrows 102 from an expanded wide profile configuration to a narrow profile configuration when passing through the ear canal and subsequently expand to the wide profile configuration when placed on one or more of the eardrum, the eardrum annulus, or the skin of the ear canal. The retention structure 110 may comprise a shape profile corresponding to anatomical structures that define the ear canal. For example, the retention structure 110 may comprise a first end 112 corresponding to a shape profile of the anterior sulcus AS of the ear canal and the anterior portion of the tympanic membrane annulus TMA. The first end 112 may comprise an end portion having a convex shape profile, for example a nose, so as to fit the anterior sulcus and so as to facilitate advancement of the first end 112 into the anterior sulcus. The retention structure 110 may comprise a second end 114 having a shape profile corresponding to the posterior portion of tympanic membrane annulus TMA.

The support structure 120 may be positioned in aperture 110A and may comprise a frame, or chassis, so as to support the components connected to support structure 120. Support structure 120 may comprise a rigid material and can be coupled to the retention structure 110, the transducer 130, the at least one spring 140, which may support transducer 130, and the photodetector 150. The support structure 120 may comprise an elastomeric bumpers 122 extending between the support and the retention structure, so as to couple the support to the retention structure 110 with the elastomeric bumpers 122. The support structure 120 may define an aperture 120A formed thereon. The aperture 120A can be sized so as to receive transducer 130, which may be, for example, a balanced armature transducer. When positioned in aperture 120A, housing 139 of the balanced armature transducer 130 may extend at least partially through the aperture 120A when transducer 130 is coupled to the tympanic membrane TM. Aperture 120A may be further sized to allow normal sound conduction through medial ear canal assembly 100.

Transducer 130 may, in embodiments of the invention, comprise structures to couple to the eardrum when the retention structure 110 contacts one or more of the eardrum, the eardrum annulus, or the skin of the ear canal. The transducer 130 may, in embodiments of the invention, comprise a balanced armature transducer having a housing 139 and a vibratory reed 132 extending out one end of housing 139. Housing 139 may also, in embodiments of the invention, be a part of a flux return path for transducer 130. In embodiments of the invention, the housing may be a fully integrated part of the transducer, including, for example, the magnetic flux path. The vibratory reed 132 may be affixed to a post 134 and an umbo pad 136. The umbo pad 136 may have a convex surface that contacts the tympanic membrane TM and may move the TM in response to signals received by medial ear canal assembly 100, causing the TM to vibrate. The umbo pad 136 can be anatomically customized to the anatomy of the ear of the user.

At least one spring 140 may be connected to the support structure 120 and the transducer 130, so as to support the transducer 130 in aperture 120A. The at least one spring 140 may comprise a first spring 142 and a second spring 144, in which each spring is connected to opposing sides of a first end of transducer 130. The springs may comprise coil springs having a first end attached to support structure 120 and a second end attached to transducer 130 or a mount affixed to transducer 130, such that the coil springs pivot the transducer about axes 140A of the coils of the coil springs and resiliently urge the transducer toward the eardrum when retention structure 110 contacts one or more of the eardrum, the eardrum annulus, or the skin of the ear canal. The support structure 120 may comprise a tube sized to receiving an end of the at least one spring 140, so as to couple the at least one spring to support structure 120.

In embodiments of the invention, a photodetector 150 may be coupled to support structure 120 of medial ear canal assembly 100. A bracket mount 152 can extend substantially around photodetector 150. An arm 154 may extend between support structure 120 and bracket mount 152 so as to support photodetector 150 with an orientation relative to support structure 120 when placed in the ear canal EC. The arm 154 may comprise a ball portion so as to couple to support structure 120 with a ball-joint 128. The photodetector 150 may be electrically coupled to transducer 130 so as to drive transducer 130 with electrical energy in response to the light energy signal radiated to medial ear canal assembly 100 by input transducer assembly 20. In embodiments of the invention, medial ear canal assembly 100 may include an electronics package 215 mounted on a back surface of photodetector 150. Electronics in electronics package 215 may be used to, for example, condition or modulate the light energy signal between photodetector 150 and transducer 130. Electronics package 215 may comprise, for example, an amplifier to amplify the signal from photodetector 150.

Resilient retention structure 110 can be resiliently deformed when inserted into the ear canal EC. The retention structure 110 can be compressed radially inward along the pivot axes 140A of the coil springs such that the retention structure 110 is compressed as indicated by arrows 102 from a wide profile configuration having a first width 110W1 as illustrated in FIG. 3 to an elongate narrow profile configuration having a second width 110W2. Compression of retention structure 110 may facilitate advancement of medial ear canal assembly 12 through ear canal EC in the direction illustrate by arrow 104 in FIG. 2 and when removed from the ear canal in the direction illustrated by arrow 106 in FIG. 2. The elongate narrow profile configuration may comprise an elongate dimension extending along an elongate axis corresponding to an elongate dimension of support structure 120 (120 W) and aperture 120A. The elongate narrow profile configuration may comprise a shorter dimension corresponding to a width of the support structure 120 and aperture 120A. The retention structure 110 and support structure 120 may be passed through the ear canal EC for placement on, for example, the tympanic membrane TM of a user. To facilitate placement, vibratory reed 132 of the transducer 130 can be aligned substantially with the ear canal EC while medial ear canal assembly 100 is advanced along the ear canal EC in the elongate narrow profile configuration having second width 110W2.

When properly positioned, retention structure 110 may return to a shape conforming to the ear canal adjacent to tympanic membrane TM, wherein the medial ear canal assembly is held in place, at least in part, by the interaction of retention structure 110 with the walls of ear canal EC. The medial ear canal assembly 100, including support structure 120, may apply a predetermined amount of force to the tympanic membrane TM when the umbo pad 136 is in contact with the eardrum. When medial ear canal assembly 100 is positioned the support structure 120 can maintain a substantially fixed shape and contact with the tympanic membrane TM is maintained, at least in part, by the force exerted by at least one spring 140.

FIG. 4 is an exploded view of a medial ear canal assembly 100 according to embodiments of the present invention which shows an assembly drawing and a method of assembling medial ear canal assembly 100. The retention structure 110 as described herein can be coupled to the support structure 120, for example, with elastomeric bumpers 122 extending between the retention structure 110 and the support structure 120. The retention structure 110 may define an aperture 110A having a width 110AW corresponding to the wide profile configuration. The support structure 120 may define an aperture 120A having a width 120AW that remains substantially fixed when the resilient retention structure is compressed. The aperture 110A of the resilient retention structure can be aligned with the aperture 120A of the support. Support structure 120 may comprise ball joint 128, and ball joint 128 can be coupled to arm 154 and bracket mount 152, such that the support is coupled to the photodetector 150.

The transducer 130 may comprise a housing 139 and a mount 138 attached to housing 139, in which the mount 138 is shaped to receive the at least one spring 140. The transducer 130 may comprise a vibratory reed 132 extending from housing 139, in which the vibratory reed 132 is attached to a post 134. The post 134 can be connected to the umbo pad 136.

The support structure 120 can be coupled to the transducer 130 with the at least one spring 140 extending between the coil and the transducer such that the umbo pad 136 is urged against the tympanic membrane TM when the medial ear canal assembly 100 is placed to transmit sound to the user. The support structure 120 may comprise mounts 126, for example tubes, and the mounts 126 can be coupled to a first end of at least one spring 140, and a second end of the at least one spring 140 can be coupled to the transducer 130 such that the at least one spring 140 extends between the support and the transducer. Umbo sensor 220 may be attached to umbo pad 136 such that umbo sensor 220 is positioned against tympanic membrane TM when medial ear canal assembly 100 is positioned in the ear canal. Umbo sensor may be positioned against any portion of the tympanic membrane and may be referred to as a tympanic membrane sensor.

FIG. 5A is an isometric top view of a medial ear canal assembly 100 according to embodiments of the invention. FIG. 5B is an isometric bottom view of a medial ear canal assembly 100 according to embodiments of the invention. In FIGS. 5A and 5B, medial ear canal assembly 100 has a retention structure 110 comprising a stiff support 121 extending along a portion of retention structure 110. The stiff support 121 may be connected to resilient member 141 and coupled to intermediate portion 149. In many embodiments, resilient member 141 and stiff support structure 120 comprise an integrated component such as an injection molded (or 3-D Printed) unitary component comprising a modulus of elasticity and dimensions so as to provide the resilient member 141 and the stiff support 121.

In the embodiments of FIGS. 5A and 5B, stiff support 121 and resilient member 141 can be conFIG.d to support output transducer 130 such that output transducer 130 is coupled to the tympanic membrane TM when the medial ear canal assembly 100, including retention structure 110 is placed in the ear canal EC. The resilient member 141 can be attached to the stiff support 121, such that the resilient member 141 directly engages the stiff support 121. The stiff support 121 can be affixed to the resilient member 141 so as to position the umbo pad 136 below the retention structure 110, such that the umbo pad 136 engages the tympanic membrane TM when the retention structure 110 is placed, for example on the tympanic membrane annulus TMA. The resilient member 141 can be conFIG.d to provide a predetermined force to the eardrum when the medial ear canal assembly 100 is placed in the Ear Canal.

In the embodiments of FIGS. 5A and 5B, resilient member 141 may comprise a resilient cantilever beam. In these embodiments, photodetector 150 may be attached to the output transducer 130 with a mount 153. Photodetector 150 and output transducer 130 can deflect together when the biasing structure 149, for example a spacer, is adjusted to couple the output transducer 130 and the umbo pad 136 to the tympanic membrane TM.

Sulcus sensors 230 may be positioned on layer 115 of retention structure 110 such that sulcus sensors 230 are in contact with the tympanic membrane TM and/or other portions of the ear canal EC when medial ear canal assembly 100 is positioned in the ear canal. Sulcus sensors 230 may also be positioned on sulcus flanges 235 to optimize their position in ear canal EC, such as, for example, to optimize their position against the tissue of tympanic membrane TM and/or against the tissue of the tympanic membrane annulus TMA. Sulcus flanges 235 may be used to, for example, position sulcus sensors 230 over regions of highly vascular tissue in the ear canal EC, such as on the tympanic membrane TM. Sulcus flanges 235 may be used to, for example, position sulcus sensors 230 over the pars tensa.

FIG. 6 shows an isometric view of the medial ear canal assembly 100. Medial ear canal assembly 100 comprises a retention structure 110, a support structure 120, a transducer 130, at least one spring 140 and a photodetector 150. Medial ear canal assembly 100 may include data processor 200 and transmitter 210 which may be positioned on transducer 130. Medial ear canal assembly 100 may further include non-contact sensors 260 and tethered sensors 250. Non-contact sensors 260 and tethered sensors 250 may be connected to data processor 200 to provide data to data processor 200. Alternatively, or in combination, one or more of data processor 200, transmitter 210, non-contact sensor(s) 260 and tethered sensors 250 may be part of, located on, or connected to electronics package 215 on photodetector 150. Tethered sensors 250 may be positioned against the skin SK in the ear canal EC where umbo sensors 220 (not shown in FIG. 6) and sulcus sensors 230 (not shown in FIG. 6) cannot contact. Alternatively or in combination, one or more of non-contact sensors 260 may be positioned loosely in ear canal EC to gather data. Retention structure 110 is sized to couple to the tympanic membrane annulus TMA and at least a portion of the anterior sulcus AS of the ear canal EC. With respect to the remaining elements of the retention structure and their function, see the discussion of FIGS. 2 and 3.

FIG. 7 shows and isometric view of the medial ear canal assembly 100 including retention structure 110, support structure 120, springs 140, a photodetector 150, and at least one drug delivery device. In embodiments of the invention, medial ear canal assembly 100 may include reservoir 400 and delivery tube 410 which are adapted to deliver drugs to the wearer. In embodiments of the invention, reservoir 400 may be used to store drugs for delivery to, for example, the tympanic membrane. In embodiments of the invention, delivery tube 410 may be used to transport drugs from reservoir 400 to umbo pad 136 which may be constructed to transmit the drugs to or through at least a portion of the tympanic membrane TM. In embodiments of the invention, umbo pad 136 may be constructed to include, for example, needles or microneedles through which drugs may be transported into the tissue of, for example, the tympanic membrane.

In embodiments of the invention, the medial ear canal assembly 100 may include sensors, such as, for example, umbo sensors 220, sulcus sensors 230 and tethered sensors 250, such as those shown in FIGS. 4, 5, and 6. In embodiments of the invention, sensors located on medial ear canal assembly 100 may be used to collect data on the user, which user data may be used to regulate the flow of drugs from the at least one drug delivery device which is incorporated into medial ear canal assembly 100.

FIG. 8 shows a lateral ear canal assembly 12, including a retention structure 310 (which may also be referred to as an ear tip retention structure) conFIG.d for placement in the ear canal. Retention structure 310 may comprise a molded tubular structure having the shape of the ear canal. Retention structure 310 may be conFIG.d to retain lateral ear canal assembly 12 in the ear canal. Lateral ear canal assembly 12 may include a signal source 320 such as a laser diode. An outer surface 340 of retention structure 310 may include ear tip sensors 240, which may be positioned against the skin SK of the ear canal EC and, alternatively or in combination, sensors (not shown) which are positioned on the medial or lateral ends of lateral ear canal assembly 12, such as, for example, a body temperature sensor.

FIG. 9 is an isometric Top view of a medial ear canal assembly in accordance with embodiments of the present invention. In FIG. 9, medial ear canal assembly 100 comprises transducer 130, photodetector 150, spring 140, support structure 120 and retention structure 110. In the embodiment of FIG. 9, sulcus sensors 230 may be positioned on retention structure 110, which may be, for example a flexible material adapted to conform to the anatomy of the user's ear canal. Retention structure 110 may comprise a material such as Parylene or Silicone.

FIG. 10 is an isometric bottom view of a medial ear canal assembly in accordance with embodiments of the present invention. In FIG. 10, medial ear canal assembly 100 may comprise transducer 130, photodetector 150, spring 140, support structure 120, retention structure 110 and umbo pad 136. In the embodiment of FIG. 10, sulcus sensors 230 may be positioned on retention structure 110, which may be, for example a flexible material adapted to conform to the anatomy of the user's ear canal. In the embodiment of FIG. 10, umbo sensors 220 may be positioned on umbo pad 136. Retention structure 110 may comprise a material such as Parylene or Silicone.

In embodiments of the invention, umbo sensors 220, sulcus sensors 230, eartip sensors 240, and tethered sensors 250 may comprise sensors that contact the skin to detect biometric data. Alternatively or in combination, umbo sensors 220, sulcus sensors 230, eartip sensors 240, and tethered sensors 250 may comprise sensors that do not require skin contact to detect biometric data. Non-contact sensors may also be sensors which do not require skin contact to detect biometric data.

Skin contacting sensors adaptable for use in embodiments of the present invention may include: micro-sensors, electrochemical sensors; thin film sensors; pressure sensors; micro-needle sensors, capacitive sensors thermometers, thermocouples, trigeminal nerve monitors; piezoelectric sensors; electrodes, pulse oximetry sensors, glucose meters, oxygen sensors and iontophoresis electrodes.

Non-skin contacting sensors adaptable for use in embodiments of the present invention may include: light sensors (e.g. optical sensors or infrared sensors); sound sensors (e.g. a microphone to pick up sounds in the ear canal); vibration sensors; heat sensors, micro-sensors; electrochemical sensors; thin film sensors; liquid (e.g. oil) sensors; accelerometers, microphones; gyroscopes, including 3-axis accelerometers, 3 axis gyroscopes; MEMS sensors, including 3 axis MEMS sensors; GPS circuitry; pedometers; reservoir monitors; walking gait sensors; battery state monitors; energy level monitors; and strain gauges.

In embodiments of the present invention, a suitable microphone might be transducer 130 wired to measure back electromagnetic fields (back EMF) which is generated when post 134 is moved independent of any drive signal provided to transducer 130, such as by vibrations in the tympanic membrane TM resulting from, for example the user speaking or snoring. The back EMF could then be provided to data processor 200 where it could be analyzed and transmitted to a receiver in lateral ear canal assembly 12 or in a remote receiver (e.g. a smart phone) by transmitter 210. In one embodiment of the invention, data processor 200 could include circuitry used to separate sounds coming from sources other than the user from sounds generated by the user to provide filtered data, which filtered feedback data may represent, for example, the user's voice.

In embodiments of the invention, a suitable optical sensor may comprise an infrared transmitter and infrared receiver. In embodiments of the invention, a suitable optical sensor may include an optical receiver tuned to the same frequency as signal source 320.

In embodiments of the invention, sensors may be 3D printed on or as an integral part of structures in the components of hearing system 10. In embodiments of the invention, non-skin contacting sensors may be mounted on, for example, the back side of photodetector 150.

In embodiments of the invention, a light may be mounted on medial ear canal assembly 100 and positioned to shine through tympanic membrane TM to illuminate the middle ear and the contents thereof. In embodiments of the invention, a sensor may be further included on medial ear canal assembly 100 to measure light reflected from the middle ear.

In embodiments of the invention, sensors on medial ear canal assembly 100 may be used to sense the position of transducer assembly with respect to structural features of the ear canal EC, such as the tympanic membrane TM. The data from such sensors may be used to position the medial ear canal assembly 100 to ensure it is properly placed and aligned in the user's ear.

In embodiments of the invention, sensors on medial ear canal assembly 100 or positioned in the ear canal EC may be used to measure environmental factors which are related to the proper functioning of the medial ear canal assembly 100, such as, degradation in photodetector output, earwax buildup, whether the user is compliant with the required oiling regimen. In embodiments of the invention, sensors may be used to ensure that the user is properly oiling by, for example, measuring the amount and regularity of oiling. In embodiments of the invention, sensors on the eartip may be used to guide and/or detect proper medial ear canal assembly insertion. In embodiments of the invention, pressure sensors and/or fluid sensors may be positioned on a medial ear canal assembly, including on the umbo pad 136 or sulcus platform to assist in the preceding tasks.

In embodiments of the invention, strain gauges may be included in the medial ear canal assembly 100 to provide feedback on the proper placement of medial ear canal assembly 100. For example, post 134 may include strain gauges which indicate when displacement starts and/or the degree of displacement by registering the lateral force on umbo pad 136. Further, the placement of one or more strain gages on retention structure 110 may provide an indication that the medial ear canal assembly 100 has lifted off of the tympanic membrane TM. In embodiments of the invention, medial ear canal assembly 100 may include features which interact with physical features of the wearer to maintain medial ear canal assembly 100 in a predetermined position in the ear canal EC, such as, for example against the tympanic membrane TM. In embodiments of the invention, such physical features may create strain on the medial ear canal assembly 100, which strain may be measured by strain gauges positioned on medial ear canal assembly 100 to ensure proper placement of medial ear canal assembly 100.

In embodiments of the invention, a feedback signal representative of the average power received by photodetector 150 may be provided, which signal may be used to quantify the coupling efficiency between photodetector 150 and signal source 320. In embodiments of the invention, the power level of signal source 320 may be adjusted to reflect the degree of coupling and the coupling efficiency indicated by the feedback signal. In embodiments of the invention, the position of lateral ear canal assembly 12 and/or medial ear canal assembly 100 may be modified to increase or decrease the level of the feedback signal, thus improving the coupling efficiency between the lateral ear canal assembly 12 and the medial ear canal assembly 100.

In embodiments of the invention, noise cancelation may be implemented by, for example, incorporating a microphone onto the back of photodetector 150. Sound signals received by the microphone could be converted into drive signals which move the tympanic membrane in opposition to the received signals such that the received signals are not perceived by the user. Such noise cancelation may be implemented such that the microphone is turned on only when the output from the photodetector exceeds a predetermined voltage, such as, for example, approximately 300 millivolts. Alternatively or in combination, the microphone may be turned on when the photodetector output voltage exceeds approximately 1 volt. In one embodiment of the invention, the sound signals may be measured by measuring the back EMF of transducer 130 and generating a signal to the transducer which causes the transducer to vibrate the tympanic membrane in a way which cancels the movement which generated the measured back EMF.

In embodiments of the present invention, sensors on medial ear canal assembly 100 or positioned in the ear canal EC may be used to measure bodily fluids, such as sweat, interstitial fluid, blood and/or cerumen (ear wax). Sensors suitable for making these measurements include electrochemical sensors, micro-needles and capacitive sensors.

In embodiments of the present invention, sensors on medial ear canal assembly 100 or positioned in the ear canal EC may be used to measure sweat for the purpose of, for example, measuring hydration levels, electrolyte balance, lactate threshold, glucose levels, calories burned, respiration rate, drug levels, metabolites, small molecules (e.g. amino acids, DHEA, cortisol, pH levels and various proteins. Sensors suitable for making these measurements include electrochemical sensors, micro-needles and capacitive sensors.

In embodiments of the present invention, sensors on medial ear canal assembly 100 or positioned in the ear canal EC may be used to measure the temperature, including the core body temperature of a user. Sensors suitable for making these measurements include thermometers, thermocouples, and optical temperature sensors. Other sensors suitable for making these measurements include resistance temperature detectors (“RTDs”), p-n junction temperature sensors. Suitable sensors may be available from Texas Instruments, including the LM94023 1.5V, micro SMD, Dual-Gain Analog Temperature Sensor with Class AB Output or the MAX31875 Low-Power I2C Temperature Sensor in WLP Package. Other sensors suitable for making these measurements include non-contact infrared temperature sensors such as the MLX90632 FIR Sensor available from Melexis. In order to make these measurements, the sensors may be placed in contact with the skin of the ear canal, by, for example, being positioned on the perimeter or sulcus platform of the contact hearing device. Sensor outputs may be used for body temperature monitoring (discrete or continuous), measuring the user's vital signs or predicting a health outcome. Measuring body temperature in the ear canal may be advantageous as it is interior to the body, nearer the core than other measurement locations.

In embodiments of the present invention, sensors on medial ear canal assembly 100 or positioned in the ear canal EC may be used to measure and/or monitor blood pressure, blood flow, heart rate, pulse, oxygen saturation levels and arrhythmia. Sensors suitable for making these measurements include electrodes, PPG (Photoplethysmography) sensors and pulse oximetry sensors. Such sensors may be positioned on or near the manubrial vessels in the ear canal. Such sensors may be positioned on or near skin covering the manubrial vessels in the ear canal.

In embodiments of the present invention, sensors on medial ear canal assembly 100 or positioned in the ear canal EC may be used to measure and/or monitor the oxygen level in a user's blood. Sensors suitable for making these measurements include optical sensors PPG (Photoplethysmography) sensors, and/or pulse oximetry sensors.

In embodiments of the present invention, sensors on medial ear canal assembly 100 or positioned in the ear canal EC may be used to measure and/or monitor drug delivery and/or medication use by monitoring the drug content in blood or interstitial fluid of a user. Sensors suitable for making these measurements include micro-needles and/or iontophoresis electrodes.

In embodiments of the present invention, sensors on medial ear canal assembly 100 or positioned in the ear canal EC may be used to measure and/or monitor body fat. Sensors suitable for making these measurements include electrodes.

In embodiments of the present invention, sensors on medial ear canal assembly 100 or positioned in the ear canal EC may be used to monitor and/or measure sleep, including the duration and/or quality of such sleep. Sensors suitable for making these measurements include accelerometers, microphones and gyroscopes.

In embodiments of the present invention, sensors on medial ear canal assembly 100 or positioned in the ear canal EC may be used to measure and/or monitor snoring and/or sleep apnea. Sensors suitable for making these measurements include accelerometers, microphones; gyroscopes; head position monitors (3 axis gyroscope); vibration sensor (microphone, TMT microactuator); oxygen sensors and trigeminal nerve monitors.

In embodiments of the present invention, sensors on medial ear canal assembly 100 or positioned in the ear canal EC and/or on the tympanic membrane may be used to measure and/or monitor the location of a user. Sensors suitable for making these measurements include GPS circuitry.

In embodiments of the present invention, sensors on medial ear canal assembly 100 or positioned in the ear canal EC may be used to measure and/or monitor the movement of a user. Sensors suitable for making these measurements include an accelerometer and/or a pedometer.

In embodiments of the present invention, sensors on medial ear canal assembly 100 or positioned in the ear canal EC may be used to measure and/or monitor calorie intake. Sensors suitable for making these measurements include microphones and piezoelectric sensors.

In embodiments of the present invention, sensors on medial ear canal assembly 100 or positioned in the ear canal EC may be used to measure and/or monitor posture, head position and/or body position. Sensors suitable for making these measurements include gyroscopes, accelerometers (including 3-axis accelerometers) and MEMS sensors (including 3 axis MEMS sensors).

In embodiments of the present invention, sensors on medial ear canal assembly 100 or positioned in the ear canal EC may be used to measure and/or monitor seizure disorders, including epilepsy, by making electroencephalogram (EEG) measurements. Sensors suitable for making these measurements include electrodes and/or electroencephalograph.

In embodiments of the present invention, sensors on medial ear canal assembly 100 or positioned in the ear canal EC may be used to measure and/or monitor electrical activities of the heart by making an electrocardiogram (ECG/EKG). Sensors suitable for making these measurements may include electrodes and/or pulse oximeters, LEDs and photodetectos. Data collected by such sensors may be used to generate electrocardiographs and/or (PPGs). Photoplethysmograms may be used to detect blood volume changes in a microvascular bed of tissue (e.g. vessels near the pars tensa and the manubrium vessels). Other information which may be measured or derived by such sensors includes heart rate, respiration, heart rate variability, rhythm abnormalities and/or blood pressure. Measured or derived information may include high blood pressure, low blood pressure, decomposition of vital signs for detection of Chronic obstructive pulmonary disease (COPD), Congestive heart failure (CHF) or stroke risk.

In embodiments of the present invention, sensors on medial ear canal assembly 100 or positioned in the ear canal EC may be used to measure and/or monitor the electrical activity produced by skeletal muscles by making an electromyogram using Electromyography (EMG). Sensors suitable for making these measurements may include electrodes and/or electromyographs.

In embodiments of the present invention, sensors on medial ear canal assembly 100 or positioned in the ear canal EC may be used to measure and/or monitor the glucose in a user's blood and/or interstitial fluid. Sensors suitable for making these measurements include glucose meters, electrochemical sensors, microneedles, and/or iontophoresis electrodes.

In embodiments of the present invention, sensors on medial ear canal assembly 100 or positioned in the ear canal EC may be used to measure and/or monitor neurological function. Sensors suitable for making these measurements may include sensors for measuring the walking gait of a user.

In embodiments of the present invention, sensors on medial ear canal assembly 100 or positioned in the ear canal EC may be used to measure and/or monitor the position and/or orientation of a user's eye.

Many other physical characteristics may be measured by sensors on medial ear canal assembly 100 or positioned in the ear canal EC, including: multi-axis acceleration; multi-axis angle; skin capacitance; infrared absorption, (e.g. pulse ox), chemical reactions; and strains.

In embodiments of the present invention, devices on medial ear canal assembly 100 or positioned in the ear canal EC may be used in combination with sensors to deliver medication to a user. Devices suitable for making these delivers may include drug reservoirs, patches, microneedles, polymers designed to elute over time and/or drug eluting materials.

In embodiments of the invention, drugs may be delivered through, for example, iontophoresis, direct skin contact, needles, drugs in the platform, drug infused silicon or other structural materials or holes or pores in the tympanic membrane structure to hold drugs prior to dispensing or weep over time.

In embodiments of the present invention, devices on medial ear canal assembly 100 or positioned in the ear canal EC may be used to stimulate serotonin production in a user by, for example, shining light in the ear canal EC for predetermined periods of time. Alternatively, such devices may be adapted to increase the production of vitamin D.

In embodiments of the present invention, devices, including sensors on medial ear canal assembly 100 or positioned in the ear canal EC may be used to recognize the speech of a user. Devices suitable for making these delivers may include microphones and speech recognition/signal processing chips and software.

In embodiments of the present invention, sensors on medial ear canal assembly 100 or positioned in the ear canal EC may be used to control the function of hearing system 10. The function of hearing system 10 may be controlled by, for example, sensing control instructions from the user, including, verbal instructions and/or instructions conveyed by finger snapping, bone conduction and/or bringing a hand or finger into proximity with the sensors on medial ear canal assembly 100. Sensors suitable for such control functions may include touch sensors, bone conduction sensors and proximity sensors. Additional sensors may include laser vibrometers, microactuators (measuring, for example, reverse EMF) or microphones. In particular any sensor adapted to measure vibration may be used. Such sensors may be positioned, for example, in the ear canal, such as, for example, on the anterior wall of the sulcus. Such sensors may be positioned on a contact hearing device. Such sensors may be adapted to measure the user's voice, internal sounds from the user's body, the user's heart rate or sounds from the user's digestive system. Such sensors may be used to diagnose or monitor conditions such as sleep apnea. Sensed signals from such sensors may be used to treat autophony by, for example, measuring the autophony and cancelling the measured autophony by sending a cancelation signal through the contact hearing device.

In embodiments of the present invention, the power required to operate sensors, drug delivery, and/or other devices located on medial ear canal assembly 100 may be supplied by one or more of the following: AC or DC current from photodetector 150; AC or DC current from an RF antenna located on or connected to medial ear canal assembly 100; Energy from a battery, micro-battery and/or super capacitor on or connected to medial ear canal assembly 100. In further embodiments of the present invention, circuitry on medial canal assembly 100 may be obtained by, for example: harvesting power from the motion of the user, including the dynamic motion of the wall of an outer ear, using, for example, a spring located on or connected to medial ear canal assembly 100; harvesting power from the motion of the tympanic membrane, including harvesting sound energy which vibrates the tympanic membrane; harvesting power from the motion of the tympanic membrane, including harvesting sound energy below approximately 100 Hz; harvesting power from the action of muscles in or near the ear canal, such as, for example muscles used in chewing food; harvesting power from the temporomandibular joint; using the movement of the eardrum (such as, for example, driven by music) to act as a pump. In embodiments of the invention circuitry on medial ear canal assembly 100 may be powered by, for example, the use of light based earplugs which transmits energy to medial ear canal assembly 100 to power the assembly when lateral ear canal assembly 12 is not being used. In embodiments of the invention, such light based earplugs may be used to recharge batteries or super capacitors located on or connected to medial ear canal assembly 100. In embodiments of the invention circuitry on medial ear canal assembly 100 may be powered by, for example, a wand which radiates, for example, RF energy to an antenna located on or connected to medial ear canal assembly 100 to power sensors on medial ear canal assembly 100 and/or in the ear canal EC for the purpose of making measurements.

In embodiments of the present invention, sensors located on medial ear canal assembly 100 may communicate data to any one of a number of devices, including lateral ear canal assembly 12, a smartphone, a smart watch, a cellular network, a ZigBee network, a Wi-Fi network, a WiGi-G network, and/or a Bluetooth enabled device. In embodiments of the present invention, such information may be transmitted from medial ear canal assembly 100 to lateral ear canal assembly 12 and from lateral ear canal assembly 12 to a smartphone, a smart watch, a cellular network, a ZigBee network, and/or a Bluetooth enabled device. In embodiments of the invention, such sensors a part of a closed loop communication network. In embodiments of the invention, communication to medial ear canal assembly 100 may be facilitated by the positioning of an antenna on or connected to medial ear canal assembly 100. In embodiments of the invention, such antennas may be printed on or formed as part of a chassis of medial ear canal assembly 100. In embodiments of the present invention, communication of data may be facilitated by the inclusion of transmitter 210 on medial ear canal assembly 100.

In embodiments of the invention, removable portions of hearing system 10 may sense emergency situations, such as fire alarms, and communicate with the user wearing medial ear canal assembly 100 using an antenna located on or connected to medial ear canal assembly 100 to warn the user of danger.

In embodiments of the invention, data collected from sensors located on medial ear canal assembly 100 or in the ear canal EC of a user may be communicated to the user's physician and/or family. In embodiments of the invention, data collected from sensors located on medial ear canal assembly 100 or in the ear canal EC of a user may be used to generate data or reports which may be communicated to the user's physician and/or family. In embodiments of the present invention, information, data or reports which may be communicated to the user, the user's physician and/or family may include information on the user's environment, including time of day, activity, surrounding sounds. In embodiments of the present invention, information, data or reports which may be communicated to the user, the user's family physician, and/or family may include information on biometric date related to the user, including blood pressure, heart rate, glucose levels, and other biometric data. In embodiments of the present invention, information, data or reports which may be communicated to the user, the user's family physician and/or family may include information on specific events related to the user or the user's physical condition, including, falls, blood pressure spikes, heart attacks, temperature spikes, impending or actual seizures, changes in specific biomarkers, or other metrics. In embodiments of the present invention, information, data or reports which may be communicated to the user, the user's family physician and/or family may include algorithm results transmitted when trends or parameters in the user's biometric data become concerning. In embodiments of the present invention, information, including warnings may be communicated to the user may include, sleep apnea warnings, drowsiness warnings (e.g. when driving), warnings of impending seizures, migraine headaches warnings, and/or cluster headache warnings.

In embodiments of the invention, medial ear canal assembly 100 may be used to communicate with the user to, for example, remind the user when to drink or when the user's sugar levels are spiking or dropping.

In embodiments of the present invention, data or other information may be transmitted by a user to the hearing system 10 of a second user. In embodiments of the invention, a user may transmit data or other information to a network of hearing systems 10.

In embodiments of the present invention, data collected by sensors positioned on medial ear canal assembly 100 or in the ear canal of a user may be collected and analyzed, by, for example, an Application on the user's smart phone. Such data may be used for many purposes, including predicating changes in the user's health and generating event alarms. Event alarms generated from the collected data might include alarms related to epilepsy seizures, migraines, cluster headaches, or predetermined changes in key biometric data or trends. Such data may be further processed to allow the user to, for example, view the data which is most important to the user, perform trend analysis on the data, correlate specific data with activities or environment, provide a dashboard of data or chart specific data. Data may also be stored for review at future doctor's appointments. Data trends may also be stored and analyzed over time.

Embodiments of the present invention are directed to a hearing system comprising a medial ear canal assembly including a transducer conFIG.d to be positioned on the tympanic membrane of a user; a lateral ear canal assembly including a signal source conFIG.d to be positioned in the ear canal of a user; and sensors connected to the medial ear canal assembly, the sensors being connected to a transmitter. In embodiments of the invention, the sensors may include sensors adapted to detect biometric data. In embodiments of the invention, the sensors may include sensors adapted to detect one or more physical characteristics of the user. In embodiments of the invention, at least one of the sensors may comprise a microphone. In embodiments of the invention, the microphone may comprise a micro-actuator. In embodiments of the invention, sound received by the micro-actuator is conFIG.d to be converted to a back EMF signal. In embodiments of the invention, the hearing system may include a data processor which is conFIG.d to convert the back EMF to a signal representative of the sound received by the micro-actuator. In embodiments of the invention the hearing system may be conFIG.d to transmit the signal representative of the sound received by the microactuator to a receiver external to the hearing system. In embodiments of the invention, the receiver comprises a smart phone, a wireless network, or a peripheral device. In embodiments of the invention, at least one of the sensors comprises a skin contacting sensor or a non-skin contacting sensor. In embodiments of the invention, at least one of the sensors comprises an umbo sensor, an eartip sensor, or a tethered sensor.

Embodiments of the present invention are directed to a method of sensing physical characteristics of a hearing system user, the hearing system comprising a medial ear canal assembly positioned on or near the tympanic membrane, the medial ear canal assembly comprising transducer sensors and a transmitter, the method comprising the steps of using the sensors to measure biometric data of the user; and transmitting the measured biometric data using the transmitter. In embodiments of the invention the method further comprising using the sensors to measure one or more physical characteristics of the user. In embodiments of the invention at least one of the sensors comprises a microphone the method further comprising the steps of measuring sound in the user's ear canal. In embodiments of the invention the microphone comprises a micro-actuator, the method further comprising measuring the back EMF signal. In embodiments of the invention the hearing system includes a data processor, the method further including the step of converting the back EMF signal to an electrical signal and transmitting the electrical signal to the data signal processor. In embodiments of the invention the back EMF signal includes a first signal portion representative of the signal received from the hearing system and a second signal representative of at least one physical characteristic of the user, the method further including the step of separating the first signal from the second signal. In embodiments of the invention the method further includes the step of transmitting the signal to a receiver external to the hearing system. In embodiments of the invention the receiver comprises a smart phone. In embodiments of the invention at least one of the sensors comprises a skin contacting sensor or a non-skin contacting sensor. In embodiments of the invention at least one of the sensors comprises an umbo sensor, an eartip sensor, or a tethered sensor. In embodiments of the invention the output transducer is used as a sensor. In embodiments of the invention the sensor is used as a microphone to measure received sound at the tympanic membrane. In embodiments of the invention the signal from the microphone is coupled to the transmitter.

Embodiments of the present invention are directed to an ear canal platform comprising: a medial ear canal assembly positioned on or over the tympanic membrane of a user; and sensors connected to the signal output transducer, the sensors being connected to a transmitter. In embodiments of the invention the sensors include sensors adapted to detect biometric data. In embodiments of the invention the sensors include sensors adapted to detect one or more physical characteristics of the user. In embodiments of the invention at least one of the sensors comprises a microphone. In embodiments of the invention the microphone comprises a micro-actuator. In embodiments of the invention sound received by the micro-actuator is conFIG.d to be converted to a voltage representative of the back EMF generated in the microactuator by the sound received by the microactuator. In embodiments of the invention the hearing system includes a data processor which is conFIG.d to convert the voltage to a signal representative of the sound received by the micro-actuator. In embodiments of the invention the signal is conFIG.d to be transmitted by the hearing system to a receiver external to the hearing system. In embodiments of the invention the receiver comprises a smart phone, a wireless network, or a peripheral device. In embodiments of the invention at least one of the sensors comprises a skin contacting sensor or a non-skin contacting sensor. In embodiments of the invention at least one of the sensors comprises an umbo sensor, an eartip sensor, or a tethered sensor.

Embodiments of the present invention are directed to a method of sensing physical characteristics of a user having a medial ear canal assembly positioned on or near the tympanic membrane, the medial ear canal assembly comprising sensors and a transmitter, the method comprising the steps of: using the sensors to measure biometric data of the user; and transmitting the measured biometric data using the transmitter. In embodiments of the invention the method further comprising using the sensors to measure one or more physical characteristics of the user. In embodiments of the invention at least one of the sensors comprises a microphone the method further comprising the steps of measuring sound in the user's ear canal. In embodiments of the invention the microphone comprises a micro-actuator, the method further comprising measuring and transmitting the output of the microphone. In embodiments of the invention the hearing system includes a data processor, the method further including the step of sending the transmitted signal to the data processor. the transmitted signal includes a first signal portion representative of the signal received from the hearing system and a second signal representative of a physical characteristic of the user, the method further including the step of separating the first signal from the second signal. In embodiments of the invention the method further includes the step of transmitting the signal to a receiver external to the hearing system. In embodiments of the invention the receiver comprises a smart phone. In embodiments of the invention at least one of the sensors comprises a skin contacting sensor or a non-skin contacting sensor. In embodiments of the invention at least one of the sensors comprises an umbo sensor, an eartip sensor, or a tethered sensor. In embodiments of the invention the output transducer is used as a sensor. In embodiments of the invention the sensor is used as a microphone to measure received sound at the tympanic membrane. In embodiments of the invention the signal from the microphone is coupled to the transmitter.

Embodiments of the present invention are directed to an ear canal platform comprising: a medial ear canal assembly positioned on the tympanic membrane of a user; a drug delivery device mounted on the ear canal assembly. In embodiments of the invention an ear canal assembly further includes sensors connected to the ear canal assembly, the sensors being connected to a transmitter. In embodiments of the invention the sensors include sensors adapted to detect biometric data. In embodiments of the invention the sensors include sensors adapted to detect one or more physical characteristics of the user. In embodiments of the invention at least one of the sensors is a microphone. In embodiments of the invention the microphone is a micro-actuator. In embodiments of the invention sound received by the micro-actuator is converted to a transmitted signal. In embodiments of the invention the hearing system includes a data processor which converts the transmitted signal to a signal representative of the sound received by the micro-actuator. In embodiments of the invention the signal is transmitted by the hearing system to a receiver external to the hearing system. In embodiments of the invention the receiver is a smart phone, a wireless network, or a peripheral device. In embodiments of the invention at least one of the sensors comprises a skin contacting sensor, or a non-skin contacting sensor. In embodiments of the invention at least one of the sensors comprises an umbo sensor, an eartip sensor, or a tethered sensor.

In embodiments of the invention umbo sensors 2220, sulcus sensors 2250, eartip sensors 2240 and/or tethered sensors 2250 may be referred to as sensors. In embodiments of the invention such sensors may be used for measuring pulse oxidation, pulse oximetry, oxygen saturation, blood pressure and/or heart rate. In embodiments of the invention a sensor may be an electrocardiogram (ECG) sensor making electrical measurements (e.g. through an electrode) wherein information derived from the sensor measurement may include heart rate, respiration, heart rate variability and/or rhythm abnormalities. In embodiments of the invention a sensor may be a photoplethysmogram (PPG) sensor making optical (e.g. with LEDs or Photodetector) measurements wherein information derived from the sensor measurements may include heart rate, respiration, heart rate variability and/or rhythm abnormalities. In embodiments of the invention a sensor may be an electroencephalography (EEG) sensor making electrical measurements (e.g. through an electrode) wherein the information derived from the sensor measurements may include brain activity. In embodiments of the invention a sensor may be an Electromyography (EMG) sensor making electrical measurements (e.g. through an electrode) wherein the information derived from the sensor measurements may include muscle activity. In embodiments of the invention a sensor may be an accelerometer measuring acceleration (e.g. using a MEMS/micro electromechanical system device) wherein the information derived from the sensor measurements may include step counting, activity classification and/or fall detection. In embodiments of the invention a sensor may be an impedance sensor making electrical measurements (e.g. through an electrode) wherein the information derived from the sensor measurements may include user activity, perspiration, respiration, hydration and/or fluid status. In embodiments of the invention a sensor may be an impedance tomography sensor making electrical measurements (e.g. through an electrode) wherein the information derived from the sensor may include hydration, fluid status and/or lactation. In embodiments of the invention the sensors may provide continuous monitoring or monitoring at predetermined, scheduled intervals or when requested by the user or a third party (e.g. a physician).

In embodiments of the invention the sensors may be PPG sensors adapted to measure PPG and/or acceleration. Such sensors may include optical physiological sensor modules and/or optical sensors modules available from Valencell, Inc, Sonion Inc. and/or ams AG (formerly known as austriamicrosystems AG). Measurements from such sensors may be used to measure and/or calculate physiologic characteristics of the user such as, heart rate, heart rate variability, rate of oxygen consumption e.g. during exercise (VO2), steps, energy expenditure, metabolic rate, cardiac efficiency and/or blood pressure.

In embodiments of the invention the sensors may be PPG sensors, ECG sensors or accelerometers or a module which includes all of those sensors and may be used with algorithms such as the algorithms available from First Beat Technologies Oy. Measurements from such sensors may be used to measure and/or calculate physiological characteristics of the user such as sleep, recovery, stress, activity, VO2, caloric expenditure and/or respiration rate.

In embodiments of the invention the sensors may be PPG sensors or accelerometers or a module which includes both of those sensors and may be used with algorithms such as the algorithms available from the Swiss Center for Electronics and Microtechnology (CSEM). Measurements from such sensors may be used to measure and/or calculate physiological characteristics of a user such as blood pressure, posture, activity, step count, cadence, speed/pace, distance traveled, energy expenditure and/or sleep profiling. Such sensors may be used to classify activities in which the user is engaged.

In embodiments of the invention the sensor may be an accelerometer and may be used with algorithms such as the algorithms available from BioSensics LLC which may be used to measure and/or calculate physiologic characteristics of a user such as physical activity, fall detection, gait and balance.

In embodiments of the invention the sensor may be an ECG sensor and may be used with algorithms such as the algorithms available from available from Monebo Technologies, Inc., which may be used to measure and/or calculate physiological characteristics of a user such as heart rate, QRS duration, PR intervals and QT intervals.

In embodiments of the invention transducer 2130 may be a piezoelectric transducer.

In embodiments of the invention, contact hearing systems 110 and hearing system 2010 may include ear to ear communication to facilitate communication between, for example, processors (132/2024). In embodiments of the invention, information obtained from one or more sensors may be communicated between systems on each ear of the user, to, for example, compare physiologic status at the left and right ear and/or tympanic membrane of the user. Such signals may be used to measure the user's EEG or EKG.

In embodiments of the invention the sensors described herein may be used to detect audio from the user by, for example, detecting audio transmitted through the user's skeletal system (bone conduction). In embodiments of the invention such audio detected through bone conduction may be transmitted to an audio processor and/or to a communication device such as a cellular telephone.

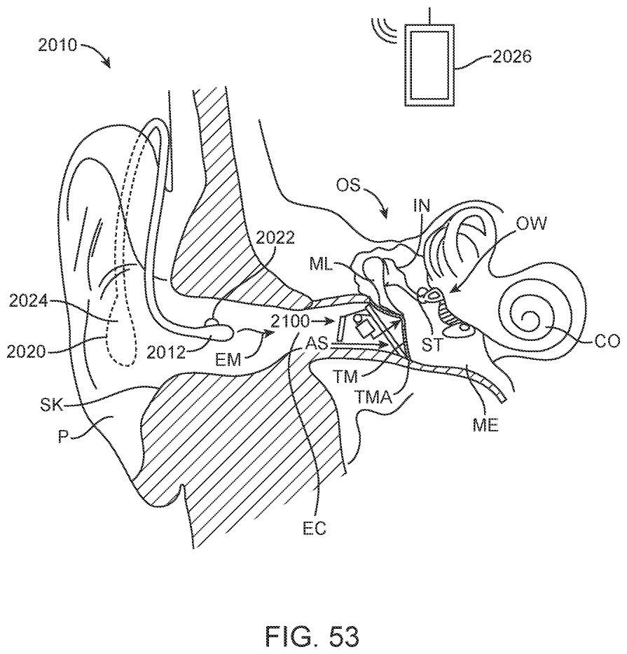

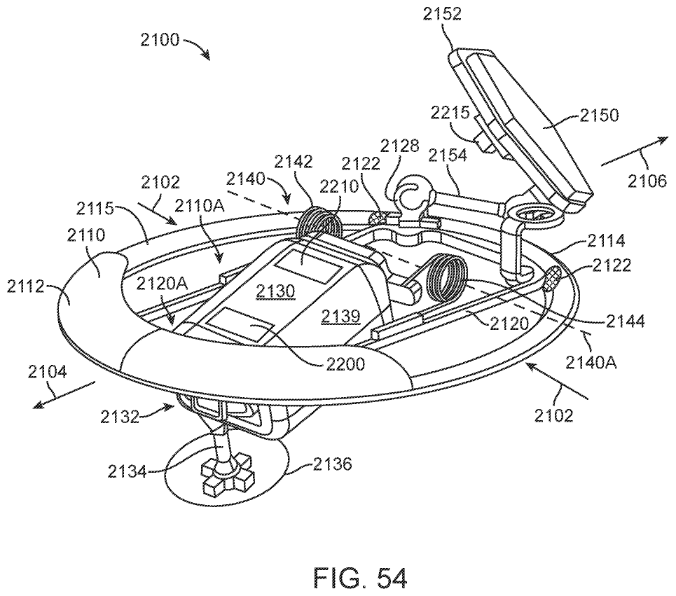

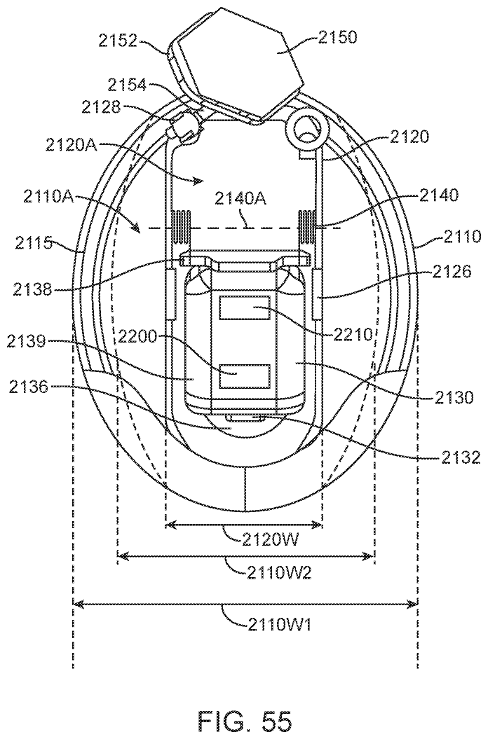

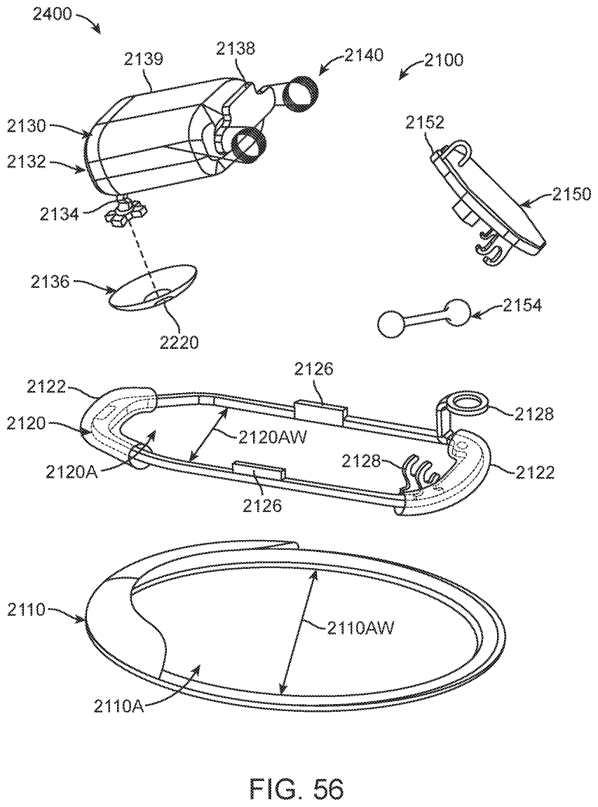

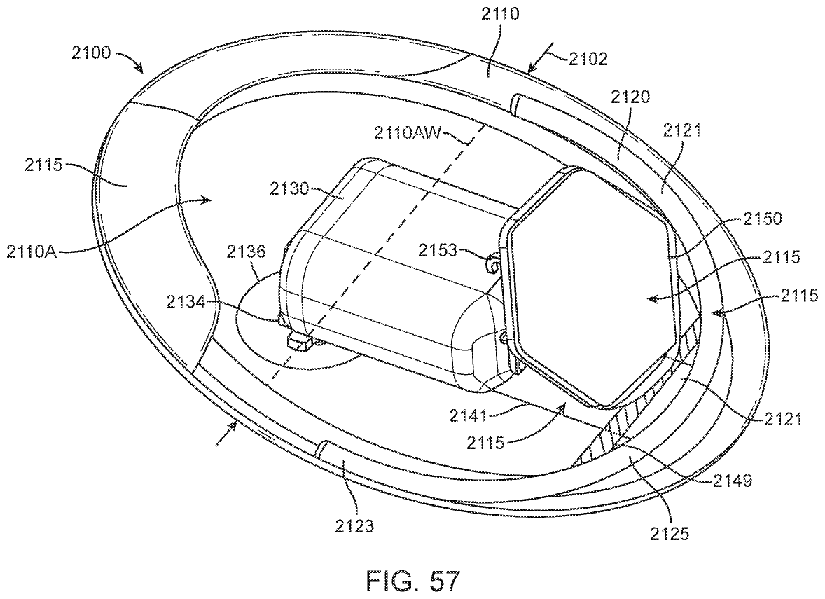

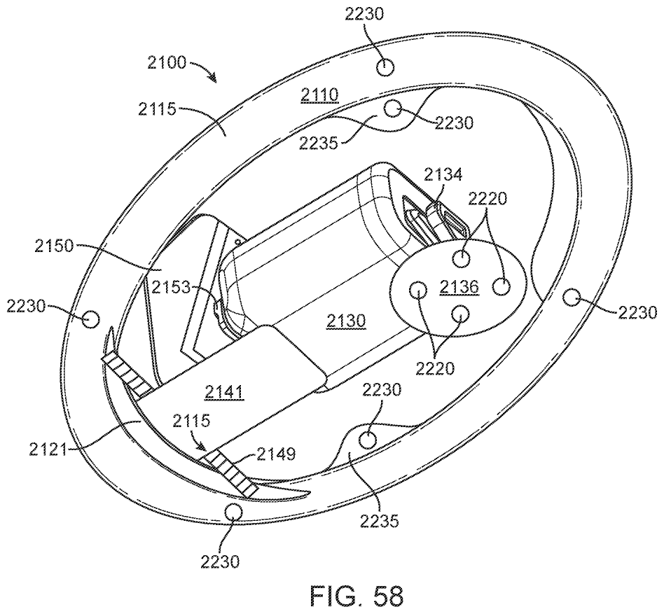

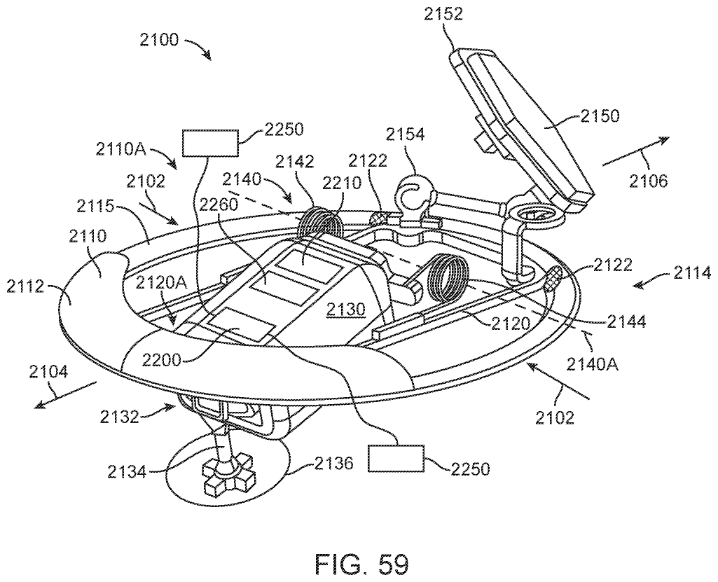

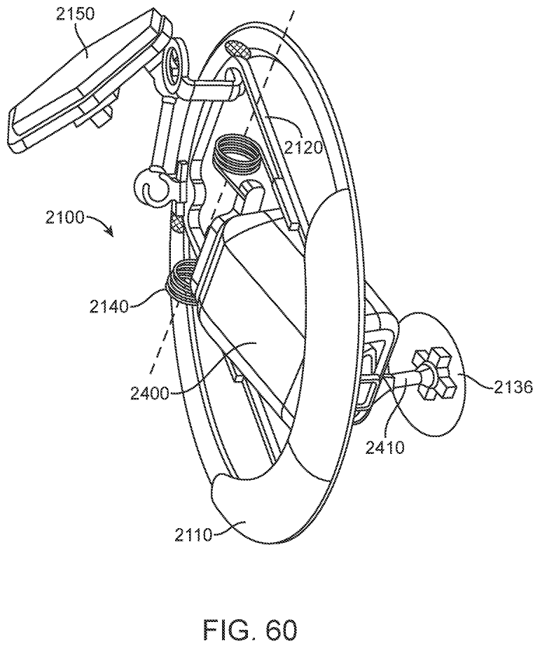

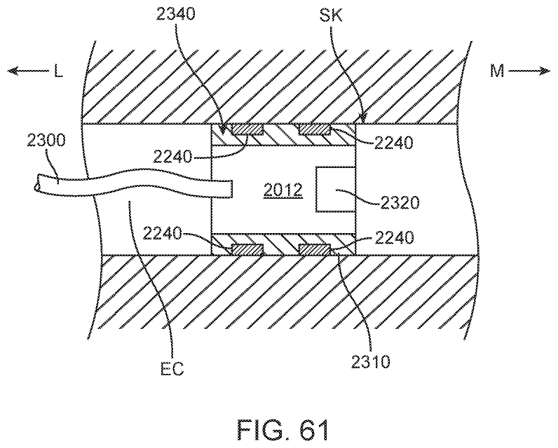

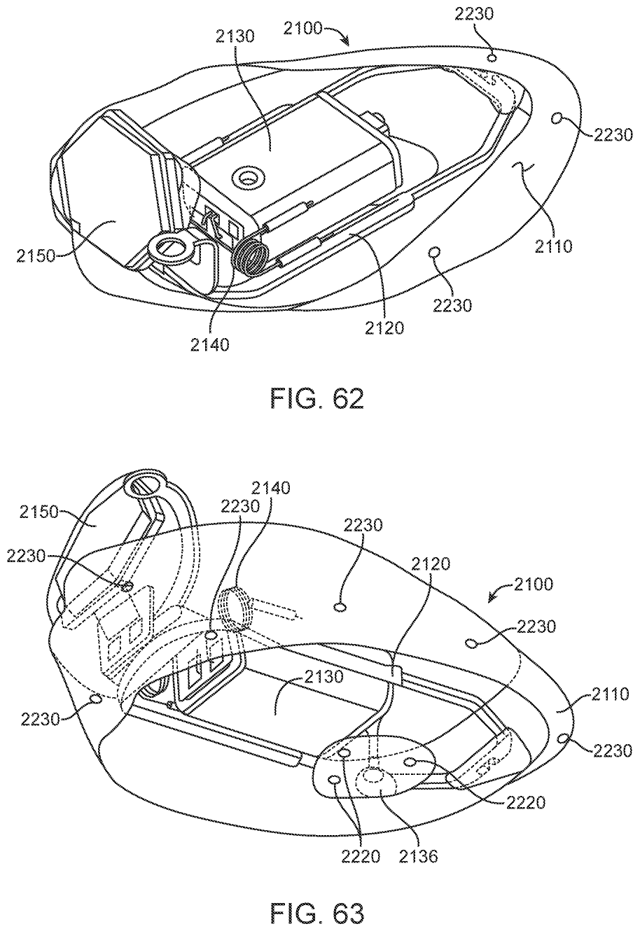

In embodiments of the invention umbo sensors 2220, sulcus sensors 2250, eartip sensors 2240 and/or tethered sensors 2250 may be positioned on elements of the systems described and illustrated in FIGS. 1-52 in the same manner as they are positioned on elements of the systems described and illustrated in FIGS. 53-63.

In embodiments of the invention, information and/or data, including physiological data, may be collected using the sensors described herein. In embodiments of the invention such information or data may be collected from the user's ear canal and/or structures adjacent to the user's ear canal. The sensors may be used to measure movement of the eardrum or the umbo. The sensors may be used to measure blood flow, including superficial blood flow by, for example, measuring blood flow at the arterioles on the surface of the eardrum or at the sulcus. The sensors may be used to measure emissions from the cochlea. The sensors may be used to measure conductive sound transmissions, for example, by monitoring the bony canal portion of the user's ear canal. Examples of sounds which may be monitored include the user's voice, respiratory sounds, sounds from the jugular vein or carotid artery and/or intestinal sounds. The sensors may be used to monitor movement of the user's head, including acceleration. Sensors may further include microactuator, which may be positioned on the contact hearing device in contact with the tympanic membrane to detect movement of the tympanic membrane in response to emissions from the cochlea, which results in a measurable back EMF at the input to the microactuator. Such sensors may be used to measure or derive drug sensitivities and/or hearing loss.

In embodiments of the invention sensors, including the sensors described herein, may be positioned to facilitate the measurements described herein. The sensors may be positioned on the anterior wall of the sulcus to facilitate certain measurements, including measuring sounds transmitted via the user's skeletal structure, such measurements being facilitated by the thin skin at the anterior wall of the sulcus. The sensors may be positioned at or near the manubrial arterioles to measure physiological characteristics related to, for example, blood flow. The sensors may be positioned at or near the circumferential sulcus to measure physiological characteristics related to, for example, blood flow. The sensors may be positioned near the sulcus and/or eardrum to measure temperature. The sensors may be positioned at or near the umbo to measure cochlear emissions.

In embodiments of the invention sensors, including the sensors described herein, may be used to measure various characteristics of the user or of the user's surroundings. The sensors may be used to measure body temperature, for the purpose of, for example, monitoring the user's health or ovulation/fertility cycles. The sensors may be used to monitor the user's heart rate. The sensors may be used to generate an EEG for the user. The sensors may be used to take a pulse oximetry measurement of the user's blood, wherein the pulse oximetry measurement may be used to, for example, calculate the user's respiration rate, heart rate or blood pressure. The sensors may be used to measure heartrate variability which may be an indication of stress. The sensors may be used to measure the user's respiration or snoring (e.g. through audio pickups), which may be an indication of sleep apnea or a means of monitoring the quality of the user's sleep. The sensed signal may also be used to generate a noise or stimulation of the tympanic membrane to awaken the user in the event of a sleep apnea event or to interrupt the sleep apnea. The monitoring of respiratory sounds may be used to detect respiratory issues. The sensors may be used to monitor the user's voice to, for example, look for changes in vocal patterns and or rhythms. Such changes could be cross correlated with blood pressure measurements and arrythmias to look for signs of impending stroke and, potentially provide a warning to the user.

In embodiments of the invention sensors, including the sensors described herein, may be used to measure glucose in a user's blood, interstitial fluid or other bodily fluids. Glucose may be measured through, for example, superficial blood vessels in the ear canal and/or below the tympanic membrane. Such measurements may be made through the skin by, for example, an optical sensor. Such measurements may be made through discrete sampling or continuous monitoring. In some measurements oil and/or retention fluid may be used to interface with blood or blood vessels in the ear canal. In some embodiments the sensor may be a patch which is adapted to detect and/or measure glucose.

In embodiments of the invention sensors, including the sensors described herein, may be used to measure uric acid in a user's blood, interstitial fluid or other bodily fluids. Uric acid may be measured through, for example, superficial blood vessels in the ear canal and/or below the tympanic membrane. Such measurements may be made through the skin by, for example, an optical sensor. Such measurements may be made through discrete sampling or continuous monitoring. In some measurements oil and/or retention fluid may be used to interface with blood or blood vessels in the ear canal. In some embodiments the sensor may be a patch which is adapted to detect and/or measure uric acid. Other measurable substances may include sweat, alcohol, ketones and/or lipids. Such measurements may be used to determine whether a user is in ketosis or the PH of the user's blood or other bodily fluids. Suitable sensors for making such measurements may include chemical sensors.

In embodiments of the invention sensors, including the sensors described herein, may be accelerometers and/or inertial sensors. In embodiments of the invention such sensors may be used to detect the gate of the user to look for changes in fore and aft sway, which may be used to predict the risk of falls and or strokes. Sensors may also be used to detect falls and or to establish the severity of a fall, by, for example, measuring acceleration and/or deceleration. The degree of acceleration and/or deceleration may also be used as an indicator of whether the user had suffered a concussion or to provide severe fall alerts.

In embodiments of the invention sensors, including the sensors described herein, may be used to measure characteristics of the contact hearing system and/or components thereof. Sensors may be used to measure displacement of components of the contact hearing system including the contact hearing device or components thereof. Sensors may be used to measure changes in the coupling or loading of components of the contact hearing system including the contact hearing device or components thereof. Sensors may measure the efficiency of communication links within the contact hearing system, including communication links between the ear tip and the contact hearing device. Such efficiency measurements could be used to tune components of the communication li not improve the efficiency of such links. Sensors may be used to measure characteristics of the ear canal such as humidity or the presence of water. Sensors may be used to measure whether there is sufficient oil (e.g. mineral oil) in the ear canal, by, for example, measuring electrical conductivity between two or more sensors.

In embodiments of the invention sensors, including the sensors described herein, may be used to detect a user's voice through, for example, bone conduction. The sensed voice may then be transmitted to an external communication device such as a cell phone which may enhance the signal to noise ratio of the transmitted voice. The sensed voice signal may be used to remove issues with the user's perception of their own voice by subtracting the measured signal from a processed signal. The sensed voice signal may be used to achieve or enhance noise canceling. In embodiments of the invention sensors may include microphones on the contact hearing device and/or on the eartip. The sensed signal may be used to provide damping, including dynamic damping to improve noise reduction. In embodiments of the invention negative gain could be applied in low frequencies (e.g. below 1 KHz) based on the phase of the signal detected at a microphone, such as a microphone positioned on an audio processor. Sensors such as accelerometers could be used to steer directional microphones in the contact hearing system by providing an indication of, for example, the direction a user is looking or the user's head position.

In embodiments of the invention sensors, including the sensors described herein, may be used to measure a number of physical characteristics. Sensors may be used to measure pressure in the middle ear. Sensors may be used to measure impedance of bones in or adjacent to the ear canal and/or the tympanic membrane. Sensors may be used to measure the absorbance and/or reflectance of the middle ear cavity. Sensor may be used to measure cochlear microphonics. Sensors may be used to measure otoacoustic emissions. Sensors may be used to measure tinnitus, including objective tinnitus. Sensors may be used to measure the frequency of tinnitus. Sensors may be used to measure sweat composition for, for example monitoring sodium or potassium or regulating drug delivery. Sensors may be used to measure galvanic skin response to measure, for example, stress, cognitive load or pain sensing.

In embodiments of the invention sensors, including the sensors described herein, may include devices or components (e.g. electrodes) which may be used to stimulate the ear canal, tympanic membrane or components thereof or adjacent structures. In embodiments of the sensors, including the sensors described herein, may be replaced by devices or components (e.g. electrodes) which may be used to stimulate the ear canal, tympanic membrane or components thereof or adjacent structures. In embodiments of the invention such devices or components may be used to stimulate the ear canal, tympanic membrane or components thereof or adjacent structures.

In embodiments of the invention the portion of the Vagus nerve which runs adjacent to the ear canal may be stimulated to treat tinnitus. The vagus nerve may be stimulated to treat depression and/or anxiety. The Vagus nerve may be stimulated to treat epilepsy and/or seizures. The Vagus nerve may be stimulated to provide parasitic control of the user's heart, lungs, and digestive tract.

In embodiments of the present invention, the present invention may have a number of applications. The present invention may be used in sports recovery. The present invention may be used to monitor the health of the user. The present invention may be used to monitor how well the contact hearing system or components thereof are working. The present invention may be used for drug detection. The present invention may be used for drug delivery. The present invention may be used in applications for normal hearing users. The present invention may be used in monitoring mental health. The present invention may be used in monitoring speech patterns. The present invention may be used in stroke detection. The present invention may be used in ototoxic monitoring for, for example, chemotherapy. The present invention may be used to detect concussions. The present invention may be used to monitor concussions. The present inventio may be used to monitor EEG by, for example, using one or more contact hearing devices as a reference position. The present invention may be used in monitoring a user's smile muscles to assist in, for example, the detection of a stroke. The present invention may be used to monitor anxiety and/or panic attacks in children or adults. The present invention may be used to monitor depression, by, for example, establishing a baseline and looking for variations from that baseline. The present invention may be used for emergency monitoring for, for example, heart attacks, strokes and/or allergic reactions. The present invention may be used for health monitoring, for, for example, earaches or infections.

In embodiments of the invention sensors, including the sensors described herein, may be used to measure general activity level as an indication of, for example, depression. The sensors may be used to monitor middle ear pressure as an indicator of, for example, cerebrospinal fluid (CSF) pressure and/or Hydrocephalus. The sensors may be uses to monitor middle ear impedance. Suitable sensors may include sensors adapted to measure dynamic impedance, or optical sensors adapted and positioned to detect the curvature of the tympanic membrane and/or changes to the curvature of the tympanic membrane. Suitable sensors may also include micro actuators positioned on the contact hearing device wherein changes in back EMF of the microactuator may be detected as an indication of changes in middle ear pressure. Information measured or sensed from such sensors may be used to diagnose ear infections, fluid accumulation or a middle ear transfer function. Such information may be used to determine the status and/or positioning of a contact hearing device in the ear canal.

Embodiments of the present invention are directed to a method of delivering drugs to a user having a medial ear canal assembly positioned on or near the user's tympanic membrane, the medial ear canal assembly comprising a drug delivery device, the method comprising the steps of: delivering drugs to the user through the drug delivery device. In embodiments of the invention the medial ear canal assembly further includes sensors and a transmitter, the method comprising the steps of: using the sensors to measure biometric data of the user; and transmitting the measured biometric data using the transmitter. In embodiments of the invention the method further includes the step of activating the drug delivery device using the biometric data measured by the sensors. In embodiments of the invention the method further comprises using the sensors to measure one or more physical characteristics of the user. In embodiments of the invention the method further comprises the step of activating the drug delivery device using the measured physical characteristics of the user. In embodiments of the invention, the step of activating drug delivery includes activating drug delivery when needed and/or at predetermined times or over predetermined time periods. In embodiments of the invention at least one of the sensors comprises a skin contacting sensor or a non-skin contacting sensor. In embodiments of the invention at least one of the sensors comprises an umbo sensor, an eartip sensor, or a tethered sensor. In embodiments of the invention, the system may comprise a reservoir and mechanisms for drug delivery.

In embodiments of the invention, the sensors described herein may be used to generate ballistocardiograms and/or sonocardiograms (phonocardiograms) for measuring heart rate, heart rate interval and heart-rate variability for the purpose of, for example, cardiac monitoring and diagnosis. In embodiments of the invention such measurements could be used to generate data related to the cardiac health of the user. Sensors suitable for taking measurements which may be used to generate ballistocardiograms and/or sonocardiograms include accelerometers (e.g. piezoelectric accelerometers and MEMS accelerometers). Sensors suitable for taking measurements which may be used to generate ballistocardiograms and/or sonocardiograms may include microphones (e.g. to measure sounds conducted by bone conduction) and gyroscopes. Such sensors may be positioned on a contact hearing device and/or components thereof.

In embodiments of the invention, sensors positioned in the ear canal may be used to monitor the movements of a user's eyes. Sensors adapted for use in making such measurements may include electrodes and/or coils. Such sensors may be adapted to detect changes in the corneal retinal potential (the standing potential that exists between the front and the back of the human eye). Such measurements may be used to detect concussions and/or intoxication.

In embodiments of the invention sensors positioned in the ear canal may be used for measuring, for example, skin impedance. Sensors suitable for making such measurements may include electrodes, which may be positioned in the ear canal and/or on the tympanic membrane. Information measured by or derived from such sensors may include levels of user activity, perspiration, respiration and/or hydration.

In embodiments of the invention sensors positioned in the ear canal may be used for measuring speech patterns of a user. Sensor's adapted to make such measurements may include bone conduction sensors to measure sound transmitted through the user's bones and/or microphones. Information sensed or derived from such sensors may be used for, for example, stroke detection.

In embodiments of the invention sensors positioned in the ear canal may include electromyography (EMG) sensors for measuring the movement of muscles adjacent the ear canal or in the face of a user.

While the preferred embodiments of the devices and methods have been described in reference to the environment in which they were developed, they are merely illustrative of the principles of the present inventive concepts. Modification or combinations of the above-described assemblies, other embodiments, configurations, and methods for carrying out the invention, and variations of aspects of the invention that are obvious to those of skill in the art are intended to be within the scope of the claims. In addition, where this application has listed the steps of a method or procedure in a specific order, it may be possible, or even expedient in certain circumstances, to change the order in which some steps are performed, and it is intended that the particular steps of the method or procedure claim set forth here below not be construed as being order-specific unless such order specificity is expressly stated in the claim.

### Definitions

Audio Processor—A system for receiving and processing audio signals. In embodiments of the invention, audio processors may include one or more microphones adapted to receive audio which reaches the user's ear. In embodiments of the invention, the audio processor may include one or more components for processing the received sound. In embodiments of the invention, the audio processor may include digital signal processing electronics and software which are adapted to process the received sound. In embodiments of the invention, processing of the received sound may include amplification of the received sound. In embodiments of the invention, the output of the audio processor may be a signal suitable for driving an inductive coil located in an ear tip. Audio processors may also be referred to as behind the ear units or BTEs.

Contact Hearing System—A system including a contact hearing device, an ear tip and an audio processor. In embodiments of the invention, contact hearing systems may also include an external communication device. In embodiments of the invention, power and/or data may be transmitted between an ear tip and a contact hearing device using inductive coupling.

Contact Hearing Device—A tiny actuator connected to a customized ring-shaped support platform that floats on the ear canal around the eardrum, where the actuator directly vibrates the eardrum causing energy to be transmitted through the middle and inner ears to stimulate the brain and produce the perception of sound. In embodiments of the invention, the contact hearing device may comprise a coil, a microactuator connected to the coil and a support structure supporting the coil and microactuator. The contact hearing device may also be referred to as a Tympanic Contact Actuator (TCA), a Tympanic Lens or a Tympanic Membrane Transducer (TMT).

Ear Tip—A structure designed to be placed into and reside in the ear canal of a user, where the structure is adapted to receive signals from an audio processor and transmit signals to the user's tympanic membrane or to a device positioned on or near the user's tympanic membrane (such as, for example, a contact hearing device). In embodiments of the invention, the signal may be transmitted using inductive coupling, using, for example, a coil connected to the Ear Tip.

Inductively Driven Hearing Aid System—a contact hearing system wherein signals are transmitted from an ear tip to a contact hearing device using inductive coupling. In an inductively driven hearing system, magnetic waves may be used to transmit information, power or both information and power from the ear tip to the contact hearing device.

Mag Tip—an ear tip adapted for use in an inductively driven hearing aid system. In embodiments of the invention, the mag tip may include an inductive transmit coil.

| Number | Element |
|---|---|
| 110 | Contact Hearing System |
| 112 | Contact Hearing Device |
| 114 | Grasping Tab |
| 116 | Demodulator |
| 118 | Sulcus Platform |
| 120 | Ear Tip/Mag Tip |
| 122 | Adhesive |
| 124 | Drive Post |
| 126 | Oil Layer |
| 128 | Charging Status LEDs |
| 130 | Receive coil |
| 132 | Audio Processor |
| 134 | AC Adapter Port |
| 136 | Charging Station |
| 138 | Charging Slots |
| 140 | Microactuator |
| 141 | Support Structure |
| 142 | Electromagnetic waves |
| 144 | Springs |
| 220 | Umbo Lens |
| 250 | Taper Tube |
| 260 | Cable |
| 290 | Transmit Coil |
| 310 | External Microphone |
| 312 | Transmit Electronics |
| 314 | Volume/Control Switch |
| 316 | Coil Winding |
| 318 | Ferrite Core |
| 312 | Canal Microphone |
| 320 | Analog to Digital Converter |
| 324 | External Communication and Control Device |
| 330 | Digital Signal Processor |
| 332 | Central Chamber |
| 334 | Mounting Recess |
| 336 | Secondary Acoustic Vent |
| 338 | Acoustic Vent |
| 340 | Acoustic Input (Audible Sound) |
| 342 | Transmit Electronics |
| 700 | Upstream Signal |
| 702 | Upstream Data |
| 710 | Downstream Signal |
| 712 | Downstream Data |
| 720 | Interface |
| 730 | Clock Recovery Circuit |
| 740 | Data Recovery Circuit |
| 750 | Energy Harvesting Circuit |
| 760 | Power management Circuit |
| 770 | Voltage Regulator |
| 780 | Driver |
| 790 | Data Processor Encoder |
| 800 | Data/Sensor Interface |
| 802 | External Antenna |
| 804 | Bluetooth Circuit |
| 806 | Battery |
| 808 | Power Conversion Circuit |
| 810 | Microphones |
| 812 | Charging Antenna |
| 814 | Wireless Charging Circuit |
| 816 | Interface Circuit |
| 818 | Power/Data Link |
| 822 | Interface Circuit |
| 823 | Biological Sensors |
| 824 | Energy Harvesting and Data Recovery Circuit |
| 826 | Energy Storage Circuitry |
| 828 | Power Management Circuitry |
| 831 | Matching Network |
| 832 | Data/Signal Processing Circuitry |
| 834 | Microcontroler |
| 836 | Driver |
| 838 | Microactuator |
| 840 | Digital Signal Processors (shown as MA in |
| | FIG. 7 appears to be wrong) |
| 842 | Cloud Based Computer |
| 844 | Cell Phone |
| 846 | Data Acquisition Circuit |
| 848 | MPPT Control Circuit |
| 852 | Current Sensor |
| 854 | Capacitor |
| 863 | Voltage Meter |
| 865 | Rectifier and Converter Circuit |
| 869 | Storage Device |
| 872 | Parasitic Capacitance |
| 882 | Load |
| 972 | Capacitor |
| 974 | Diode |
| 975 | AC Filter Capacitor |
| 976 | Input Circuit |
| 977 | Resonance Capacitor (Tuning Capacitor) |
| 978 | Output Circuit |
| 980 | Drive Coil L1 |
| 982 | Load Coil L4 |
| 984 | Resonant Transmit Coil L2 |
| 986 | Resonant Receive Coil L3 |
| 988 | Drive (Transmit) Circuit |
| 990 | Load (Receive) Circuit |
| 992 | Transmit Resonant Circuit |
| 994 | Receive Resonant Circuit |
| 996 | Signal Source |
| 998 | Resonant Transmit Capacitor C1 |
| 1000 | Resonant Receive Capacitor C2 |
| 1002 | Voltage Detector |
| 1004 | Rectifier Circuit |
| 1006 | Load |
| 1008 | Receive Inductor Lrx |
| 1010 | Receive Capacitor Cr1 |
| 1012 | Receive Capacitor Cr2 |
| 1014 | Receive Capacitor Cr3 |
| 1016 | Receive Capacitor Cr4 |
| 1018 | Diode D1 (Schottky) |
| 1020 | Diode D2 |
| 1022 | Diode D3 |
| 1024 | Diode D4 |
| 1026 | Smoothing Capacitor 1026 |
| 1028 | Motor |
| 1030 | Motor Resistor (Resistance) |
| 1032 | Motor Inductor (Inductance) |
| 1034 | Diode Bridge |
| 1036 | Transmitter |
| 1038 | Current Source |
| 1040 | Output Capacitor C0 |
| 1042 | Output Coil L1 |
| 1044 | Voltage Source |
| 1046 | Capacitive Transformer/Divider |
| 1048 | Resistor RI |
| 1050 | Capacitor C01 |
| 1052 | Capacitor C02 |
| 1054 | Inductor L1 |
| 1056 | Parallel Drive Circuit |
| 1058 | Capacitor C7 |
| 1060 | Capacitor C1 |
| 1062 | First Diode |
| 1066 | Motor Node |
| 1067 | Diode Output Node |
| 1068 | Smoothing Capacitor |
| 1070 | Second Diode |
| 1072 | Receive Circuit Components |
| 1074 | Receive Circuit Board |
| 1076 | Adhesive |
| 1078 | Ferrite Disk(s) |
| 1080 | Receive Coil Windings |
| 1082 | Adhesive Plug |
| 1084 | Receive Circuit Assembly |
| 1086 | AND/NAND Gate |
| 1088 | Switch S1 |
| 1090 | Switch S2 |
| 1092 | Switch S3 |
| 1094 | Switch S4 |
| 2010 | Hearing system |
| 2012 | Lateral ear canal assembly |
| 2020 | Input Transducer Assembly |
| 2022 | Microphone |
| 2024 | Sound Processor [Behind the Ear Unit (BTE)] |
| 2026 | External Communication Device (Cell Phone) |
| 20100 | Medial ear canal assembly |
| 2102 | Arrows |
| 2104 | Arrow |
| 2106 | Arrow |
| 2110 | Retention Structure |
| 2110A | Aperture |
| 2112 | First end (anterior end) |
| 2114 | Second end (posterior end) |
| 2115 | Layer (on retention structure 110) |
| 2120 | Support Structure |
| 2120A | Aperture |
| 2121 | Stiff Support |
| 2122 | Elastomeric bumpers |
| 2128 | Ball Joint (ball mount) |
| 2130 | Transducer |
| 2132 | Vibratory reed |
| 2134 | Post |
| 2136 | Umbo Pad |
| 2139 | Housing |
| 2140 | Spring |
| 2140A | Axis/pivot axis |
| 2141 | Resilient Member |
| 2142 | First spring |
| 2144 | Second spring |
| 2150 | Photodetector |
| 2152 | Bracket mount/bracket |
| 2154 | Arm |
| 2200 | Data Processor |
| 2210 | Transmitter |
| 2215 | Electronics package |
| 2220 | Umbo sensor(s) |
| 2230 | Sulcus sensor |
| 2240 | Eartip sensor |
| 2250 | Tethered sensors |
| 2260 | Non-contact sensors |
| 2300 | Connector (wire/optical cable etc.) |
| 2310 | Eartip Retention structure |
| 2320 | Signal Source |
| 2340 | Outer surface (of retention structure 310) |
| 2400 | Reservoir |
| 2410 | Delivery tube |
| EM | Electromagnetic energy |
| BTE | Behind the ear unit |
| EC | Ear canal |
| AS | Anterior sulcus |
| TM | tympanic membrane (eardrum) |
| TMA | Tympanic membrane annulus |
| ME | Middle ear |
| ST | Stapes |
| IN | Incus |
| OS | Ossicles |
| ML | Malleus |
| OW | Oval Window |
| CO | Cochlea |
| SK | Skin |

1. A method of measuring a biometric characteristic of a user, the method comprising the steps of:
positioning a sensor in the ear canal of a user, wherein the sensor is positioned on or connected to an ear tip, contact hearing device, or one or more components thereof, and wherein the sensor and the ear tip, contact hearing device, or one or more components thereof are in place in the ear canal of the user for 24 hours in a day;
sensing one or more biometric signals from the sensor; and
measuring or deriving the biometric characteristic of the user from the one or more biometric signals.

2. The method of claim 1, wherein the measured or derived biometric characteristic of the user is a temperature of the user, and wherein the sensor is a temperature sensor.

3. The method of claim 1, wherein the one or more biometric signals comprises one or more acoustic signals, and wherein the sensor is an acoustic sensor.

5. The method of claim 1, wherein the one or more biometric signals comprise one or more movement related signals from the sensor, and wherein the measured or derived biometric characteristic comprises a movement of the user or a portion of the user's body.

6. The method of claim 1, further comprising using the sensed one or more biometric signals to generate a ballistocardiogram for the user.

7. The method of claim 1, further comprising using the sensed one or more biometric signals to generate an electrocardiogram for the user.

8. The method of claim 1, wherein the biometric characteristic comprises an oxygen saturation level of the user.

9. The method of claim 1, wherein the measured or derived biometric characteristic comprises a blood pressure of the user.

10. The method of claim 9, wherein the sensor is a photoplethysmography sensor.

11. The method of claim 1, wherein the measured or derived biometric characteristic of the user comprises a respiration rate.

12. The method of claim 1, further comprising placing a drug delivery device in proximity to the ear canal of the user.

13. The method of claim 1, further comprising transmitting the one or more biometric signals or the measured or derived the biometric characteristic from the ear tip, contact hearing device, or one or more components thereof to a receiver.

14. The method of claim 13, wherein the receiver is a smart phone.

15. The method of claim 13, wherein the one or more biometric signals or the measured or derived the biometric characteristic is transmitted inductively.

16. The method of claim 1, wherein the sensor is positioned on a contact hearing device positioned in contact with a tympanic membrane of the user.

17. The method of claim 16, wherein the sensor is positioned to be in contact with an umbo or tympanic sulcus of the tympanic membrane of the user.

18. The method of claim 1, wherein the sensor is positioned on or connected to the ear tip, contact hearing device, or one or more components thereof to be in contact with skin of the ear canal of the user.

19. The method of claim 1, wherein the sensor is a skin contacting sensor, and wherein the skin contacting sensor is selected from the group comprising: micro-sensors, electrochemical sensors; thin film sensors; pressure sensors; micro-needle sensors, capacitive sensors thermometers, thermocouples, trigeminal nerve monitors; piezoelectric sensors; electrodes, pulse oximetry sensors, glucose meters, oxygen sensors, and iontophoresis electrodes.

20. The method of claim 1, wherein the sensor is a non-skin contacting sensor, and wherein the non-skin contacting sensor is selected from the group comprising: light sensors; optical sensors; infrared sensors; sound sensors; microphones; vibration sensors; heat sensors, micro sensors; electrochemical sensors; thin film sensors; liquid sensors; oil sensors; accelerometers, microphones; gyroscopes; 3-axis accelerometers, 3 axis gyroscopes; MEMS sensors; 3 axis MEMS sensors; GPS circuitry; pedometers; reservoir monitors; walking gait sensors; battery state monitors; energy level monitors; and strain gauges.
